(19) United States
(12) Patent Application Publication
(10) Pub. No.: US 2026/0126450 A1
(43) Pub. Date: May 7, 2026

(54) LIBRARY-SCALE METHODS FOR POLYPEPTIDE FUNCTIONAL ANALYSIS

(71) Applicants: Massachusetts Institute of Technology, Cambridge, MA (US); THE GENERAL HOSPITAL CORPORATION, Boston, MA (US)

(72) Inventors: Brandon DEKOSKY, Cambridge, MA (US); Shuyan JIN, Cambridge, MA (US); Shelbe Marie JOHNSON, Cambridge, MA (US); Matias F. GUTIERREZ, Cambridge, MA (US)

(21) Appl. No.: 19/117,910

(22) PCT Filed: Oct. 12, 2023
(86) PCT No.: PCT/US2023/076724

§ 371 Date: Apr. 2, 2025

Related U.S. Application Data
(60) Provisional application No. 63/415,514, filed on Oct. 12, 2022.

(51) Int. Cl. G01N 33/68; C12N 15/10

(52) U.S. Cl. CPC G01N 33/6845; C12N 15/1037

(57) ABSTRACT

Disclosed herein are methods, compositions, systems, and kits related to functional testing of polypeptide-target interactions, such as antigen/immune receptor interactions, in a single-cell format.

### CROSS-REFERENCE TO RELATED APPLICATIONS

This application claims the benefit of and priority to U.S. Provisional Patent Application No. 63/415,514, filed Oct. 12, 2022, the entire contents of which are incorporated by reference herein.

### SEQUENCE LISTING

A Sequence Listing accompanies this application and is submitted as an XML file of the sequence listing named “631020_00178_sequence_listing.xml” which is 36,491 bytes in size and was created on Oct. 11, 2023. The sequence listing is electronically submitted via Patent Center with the application and is incorporated herein by reference in its entirety.

### TECHNICAL FIELD

The present technology relates generally to methods and compositions useful for the analysis and screening of polypeptide:target interactions, such as the interactions between immune receptors and antigens. The methods, systems, kits, and compositions disclosed herein provide tools for rapidly, efficiently, and accurately screening polypeptide-target (e.g., immune receptor:antigen) interactions.

### BACKGROUND

A major need exists for improved and rapid assays for identifying and mapping interactions between proteins and their targets, such as between antigens and antigen-binding proteins such as antibodies and T cell receptors. Assays for determining polypeptide:target and antigen/immune receptor interactions require substantial quantities of purified polypeptides and use low- or medium-throughput (<10,000) assays to test polypeptide function in well plates. Examples include cell-based assays, viral neutralization assays, or cellular activity-based protein functional activation assays. Currently, the process of expressing, purifying, and analyzing polypeptides is not readily compatible with direct selection of functional polypeptide interactions, such as the determination of not only binding, but for antibodies also of the activation of immune responses via antibody/antigen interactions and the recruitment of other immune components.

### SUMMARY

Disclosed herein are methods, compositions, systems, and kits related to functional testing of soluble polypeptides and the determination of protein-protein or immune receptor-antigen binding in a single-cell format.

In some aspects, a screening method is provided.

In some embodiments, the screening method includes detecting the presence of binding between a secreted polypeptide and a membrane-bound target, wherein the secreted polypeptide and the membrane-bound target are included within a compartment, wherein the compartment includes a single, isolated genetically engineered cell expressing the secreted polypeptide and presenting the membrane bound target, and wherein the cell is engineered to secrete the secreted polypeptide, present the membrane bound target, or both.

In some embodiments, the screening method includes detecting binding between a secreted polypeptide and a membrane-bound target includes: providing a detection agent, wherein the detection agent binds to the secreted polypeptide, the membrane-bound target, or both.

In some embodiments of the screening method, the detection agent includes one or more of an antibody Fc effector protein, a complement protein, a ligand, a polypeptide, a chemical, a nucleic acid sequence, an antibody or fragment thereof. In some embodiments of the screening method, the detection agent includes a reporter molecule. In some embodiments of the screening method, the reporter molecule includes one or more of a nucleic acid sequence, such as a nucleic acid barcode, a dye, a fluorescent molecule, an enzyme, a chemical, a protein, a polypeptide tag.

In some embodiments of the screening method, the secreted polypeptide or the membrane-bound target includes a polypeptide sequence derived from an antibody or fragment thereof, such as from an scFv, chimeric antigen receptor (CAR), antigen binding fragment of heavy chain (VHH), or nanobody. In some embodiments of the screening method, the secreted polypeptide and/or the membrane-bound target comprises at least one of a chemical moiety, a polymer, an oligomer, a nucleic acid, and a peptide sequence, optionally a fusion protein.

In some embodiments of the screening method, the compartment is a well, a droplet, spatially separated cell culture condition, or an encapsulation. In some embodiments of the screening method, the secreted polypeptide includes a reporter molecule. In some embodiments of the screening method, the detection comprises a cell sorting step, a sequencing step, or both.

In some embodiments, the screening method includes, before or contemporaneous with the detecting step, generating a collection of genetically engineered cells, wherein each of the genetically engineered cells includes a gene encoding a secreted polypeptide from a library of secreted polypeptides.

In some embodiments, the screening method includes, before or contemporaneous with the detecting step, generating a collection of genetically engineered cells, wherein each of the genetically engineered cells includes a gene encoding a membrane-bound target from a library of membrane-bound targets.

In some embodiments, the screening method includes, before or contemporaneous with the detecting step, generating a collection of genetically engineered cells, wherein each of the genetically engineered cells includes a gene encoding a membrane-bound polypeptide of a library of membrane-bound polypeptides and a gene encoding a secreted polypeptide of a library of secreted polypeptides.

In some embodiments of the screening method, the antibody Fc effector protein includes one or more of FcγRI, FcγRIIa, FcγRIIb, FcγRIIc, FcγRIIIa, FcγRIIIb, FcαRI, FcμR, FcαR, FcμR, FcεRI, FcεRII/CD23, DC-SIGN, Fcα/μR, FcRn, other Fc effector proteins, or a fragment thereof.

In some embodiments of the screening method, the complement protein includes at least one of C1q, C1s, C1r, a C1 complex, C1 complex proteins, C2b, C4a, C4b, C3, C3a, C3b, C5, C5a, C5b, C6, C7, C8, C9, Bb, Ba, Factor D, Factor B, Factor I, Factor H, complement pathway protein fragments, binding partners, factors, enzymes, proteases, or fragments thereof.

In some embodiments of the screening method, the detection agent includes another cell or a virus (e.g., a lentivirus or a phage). In some embodiments of the screening method, the secreted polypeptide includes one of a T cell receptor and a peptide:MHC complex.

In some embodiments of the screening method, the membrane-bound target includes one of a T cell receptor, and a peptide:MHC complex. In some embodiments, the screening methods include detecting the presence of binding between the secreted polypeptide and the membrane-bound target includes detecting the presence or absence of a reporter molecule associated with the cell.

In some embodiments of the screening method, the reporter molecule is present within the cell, associated with the cell, or expressed by the cell when the secreted polypeptide binds the membrane-bound target. In some embodiments of the screening method, the detection agent includes another cell or a virus, optionally, a lentivirus or a phage.

In some embodiments of the screening method, the reporter molecule is absent or diminished when the secreted polypeptide binds the membrane-bound target. In some embodiments of the screening method, the secreted polypeptide includes protein a proteolysis targeting chimera (PROTAC). In some embodiments of the screening method, the secreted polypeptide including the PROTAC includes an antibody, scFv, VHH, nanobody, TCR, pMHC, Fab, IgG, ligand, or other binding polypeptide.

In some embodiments of the screening method, one or more of the secreted polypeptide the membrane-bound target, and the detection agent include a reporter molecule. In some embodiments of the screening method, the membrane-bound target is bound to or expressed on the cell surface prior to compartmentation. In some embodiments of the screening method, the membrane-bound target is bound to or expressed on the cell surface during or after compartmentation.

In some aspects, a kit is provided.

In some embodiments, the kit includes (a) a nucleic acid sequence encoding a secreted polypeptide or a cell containing the nucleic acid sequence encoding the secreted peptide; and (b) a nucleic acid sequence encoding a membrane-bound target, or a cell containing the nucleic acid sequence encoding the membrane-bound target, wherein the secreted polypeptide may be screened for binding to the membrane-bound target; optionally, wherein the nucleic acid encoding the secreted polypeptide and the nucleic acid encoding the membrane-bound target are present on a same construct.

In some embodiments of the kit, the kit includes a detection agent, optionally, wherein the detection agent includes a reporter molecule. In some embodiments of the kit, the detection agent includes an antibody Fc effector protein, or a complement protein.

### BRIEF DESCRIPTION OF THE DRAWINGS

FIG. 1A-1B. (FIG. 1A) SARS-CoV-2 and (FIG. 1B) HIV receptors expression plasmids used to modify cell lines to make them permissible to virus or pseudovirus entry.

FIG. 2. The generation of cells expressing ACE2 and TMPRSS2 for SARS-CoV-2 infection, which are also capable of antibody secretion, to enable large-scale compartment-based library screening for antibody SARS-CoV-2.

FIG. 3. Generating a cell line include ACE2, TMPRSS2 and IgG gene allowing neutralization assay to be performed in a single cell basis by linking protein secretion (in this case, an IgG) to viral infection along with a functional readout for infection.

FIG. 4A-4B. Cell line development for single-cell SARS-CoV-2 neutralization assays. (a) Incorporation of ACE2 and TMPRSS2 into the site-specific TARGATT cell line for transgene insertion. (b) Paired VH:VL library cloning in TARGATT cells.

FIG. 5. Evaluation of SARS-CoV-2 Pseudovirus infectivity using different amount of virus through flow cytometry analysis.

FIG. 6A-6B. Vector maps of (a) pCMV-EF1a vector and (b) pBI vector, two examples of vectors that can enable protein or peptide secretion. In this case, the secreted protein is an IgG.

FIG. 7. ELISA quantification comparison of IgG yield for transient IgG expression. Different leader peptide sequence combinations can provide different levels of secreted protein expression.

FIG. 8. The FRT/FLP based site-directed integration system for IgG expression.

FIG. 9. Integrase-based site-directed integration system for IgG expression.

FIG. 10. CRISPR/Cas9 homologous-directed repair system for IgG expression into cell lines for analysis of soluble protein function.

FIG. 11. Overview of several possible secreted protein expression platforms for library cloning into mammalian cells for secreted protein assays.

FIG. 12A-12B. Expression of IgG in a single-directional format. ( ) Expression of IgG as single-chain variable fragment. (b) Expression of full IgG in a bi-cistronic format with a p2A cleavage peptide.

FIG. 13. Neutralizing activity of VRC01 and 910-30 via flow cytometry of HEKACE2 cells.

FIG. 14A-14B. Linked antibody secretion and SARS-CoV-2 infection for neutralization assays. (FIG. 14A) ELISA standard curve for IgG secreted by HEK293-ACE2. (FIG. 14B) 96-well neutralization assays for HEK293-ACE2 cells expressing neutralizing mAbs (first, second, fifth, and sixth group) or non-neutralizing mAbs (third and fourth group), with two different leader peptides (LP4 or LP5). Secreted mAb concentration is reported above each bar. IgG-secreting cells prevented pseudovirus infection. 910-30 SARS-CoV-2 IC₅₀ is approximately 0.2 μg/mL.

FIG. 15A-15B. Single-cell isolation and antibody secretion inside emulsion droplets. (FIG. 15A) Single cells were encapsulated in 80 μm droplets and analyzed by light microscopy. (FIG. 15B) Cells were incubated and secreted antibody, either in bulk cell culture or inside droplets. Supernatants were recovered and analyzed by ELISA to determine antibody concentrations (avg. +/−st. dev.). The concentration in droplets quickly exceeded 0.5 μg/mL by Day 2. *Extrapolation slightly above the standard curve.

FIG. 16A-16C. Example of high-throughput single-cell neutralization assay for mapping natively paired human antibodies against diverse SARS-CoV-2 variants. (FIG. 16A) Single TARGATT-HEK293-ACE2 cells secreting antibodies are captured inside emulsion droplets. (FIG. 16B) After around 24 h of antibody secretion, single cell droplets are merged with SARS-CoV-2 pseudovirus droplets. Cells secreting neutralizing antibody at sufficient concentration are protected from infection. (FIG. 16C) Cells are sorted into GFP− and GFP+ populations. Non-infected GFP-cells can be passaged for multiple screening rounds. DNA amplicons of sorted libraries are recovered for quantitative analysis and subsequent antibody expression. The renewable libraries can be screened repeatedly against diverse SARS-CoV-2 pseudoviruses separately, or against pseudovirus panels, to select for broad vs. strain-specific antibodies.

FIG. 17. Yellow fever virus (YFV) neutralization detection in cells secretion anti-YFV monoclonal antibodies. Cells secreting mAb-17 were protected from YFV RVP infection; cells not expressing mAb-17 were infected by RVPs as demonstrated by expressed GFP after RVP exposure.

FIG. 18A-18D. ELISA quantification of antibody expression using different leader peptide and promoter combinations. (FIG. 18A) Table of Leader Peptide Amino Acid Sequences and Leader Peptide Pair Names. (FIG. 18B) Plasmid illustration of minimal human cytomegalovirus (miniCMV) bi-directional promoter to drive expression of heavy and light chains of antibody and dual promoters consisting of CMV to express the heavy chain of the antibody and human elongation factor-1 alpha (Eflu) driving expression of the light chain. (FIG. 18C) Sandwich ELISA quantification of VRC01 transient expression levels with different leader peptide combinations in each vector. (FIG. 18D) Sandwich ELISA quantification of CR3022 transient expression levels with different leader peptide combinations in each vector.

FIG. 19. A CRISPR-Cas9 integration system for antibody secretion in mammalian cells.

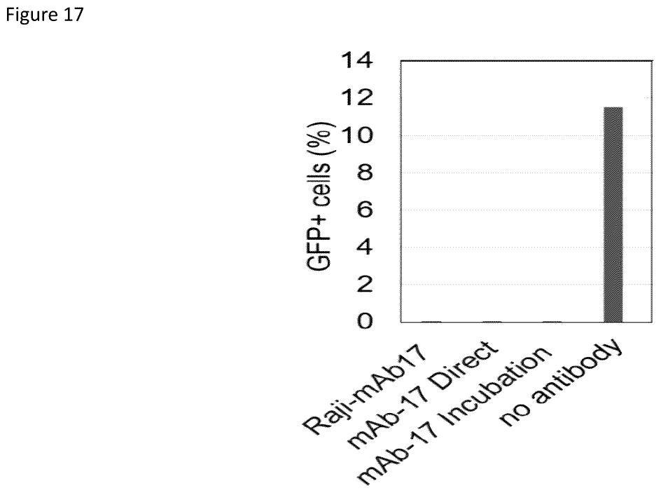

FIG. 20. Neutralization was demonstrated using CRISPR-Cas9 integration system for antibody secretion.

FIG. 21. Quantification of cell-secreted antibodies demonstrated the use of CRISPR-Cas9 to achieve antibody secretion.

FIG. 22A-22B. Verification of CRISPR-Cas9-based genomic insertion of antibody genes into mammalian cells.

FIG. 23. TARGATT gene integration of the mAb 2-15 sequence.

FIG. 24. Neutralization activity of an anti-SARS-CoV2 antibody, 2-15, secreted from the TARGATT12-15 cells.

FIG. 25. Quantification of antibody secretion from the TARGATT2-15 cells.

FIG. 26A-26B. (FIG. 26A) Gel electrophoresis of the genomic PCR using a downstream primer set to validate the successful gene-integration of TARGATT2-15 cells. (FIG. 26B) A PCR reaction using a human control primer set as an internal PCR control (panel b).

FIG. 27. HIV-1 neutralization detection in cells secreting anti-HIV-1 monoclonal antibodies. Cells secreting VRC34 were protected from HIV-1 pseudovirus infection; cells not expressing VRC34 were infected by pseudovirus as demonstrated by expressed GFP after W6M.EnV.C2 HIV-1 pseudovirus exposure. n=2 replicates were performed at each condition. The initial cell density was 2,500 cells/well. Dilutions were made with pseudovirus particles; the number of cells and antibody concentrations were held constant across pseudovirus dilutions. WT-wild type, NC-negative control (no pseudovirus particles added).

FIG. 28. Droplet merging using electrocoalescence. Top: Droplet merger is off. Droplets containing cells and droplets containing rhodamine are clearly separated, both in the bright field and when measuring rhodamine fluorescence. Bottom: Droplet merger is on using an electric field, with settings at 1.6 V. Droplet containing cells merge with rhodamine 110 dye for visibility using microscopy, as shown in rhodamine 110 channel. Arrows indicate the presence of cells inside droplets. No rhodamine is present in the cell-containing droplets when the droplet merger voltage is “off,” whereas rhodamine is present inside droplets containing cells when the droplet merger voltage is “on,” indicating successfully merged droplets.

FIG. 29. PCR amplification of variable heavy chain sequences from cell lines analyzed in high-throughput assays. Cell population libraries were sorted for GFP- or GFP+ expression prior to DNA recovery using a flow cytometer. These data demonstrate our ability to recover the DNA sequences of cells utilized in high-throughput droplet-based cell secretion protein functional assays.

FIG. 30. SARS-CoV-2 droplet neutralization assay implementation with synthetic libraries. HEK293/ACE2 cells expressing either VRC01, CR3022 910-30 or mAb 1-20 were pooled and single cells were captured and allowed to secrete antibody for 24 hours. Droplet-containing cells and antibody were merged with droplets containing SARS-CoV-2 D614G RVPs allowing infection for 24 hours. After infection, cells were recovered from the droplets and allowed. Two days later, GFP−/mCherry+ (not infected cells/mAb producing) and GFP+/mCherry+(infected cells/mAb producing) cells were sorted. gDNA was extracted from both populations for sequencing, while 10% of the recovered GFP−/mCherry+ cells were expanded for a second round of droplet neutralization assay. Zero reads were observed in GFP+ populations for some clones, reflecting a total lack of infection events for those neutralizing antibody clones and providing the expected outcome with very high assay precision. Division calculations for clonal fraction of read fold-changes, defined as (GFP-read prevalence/GFP+ read prevalence), can result in a divide by zero error when zero reads are available (indicating complete neutralization inside droplets for certain antibody clones, for example). Mathematically the closest approximation for a divide by zero error would be infinity, however those fold changes were artificially estimated here at a value of 9,999 for the purposes of comparison to other clones.

FIG. 31. Droplet neutralization assay using HIV-1 pseudovirus with synthetic libraries. TZM/GFP cells expressing either 72A1, VRC01 or VRC34 were pooled. Next, single cells were captured and allowed to secrete antibody for 24 hours. Droplet-containing cells and antibody were next merged with droplets containing HIV pseudoviruses (generated using the sequence BG505.W6M.Env.C2) allowing infection for 24 hours. After infection, cells were recovered from the droplets and allowed. Two days later, GFP−/mCherry+(not infected cells/mAb producing) and GFP+/mCherry+(infected cells/mAb producing) cells were sorted. gDNA was extracted from both populations. The non-neutralizing antibody (72A1) was greatly enriched in the GFP+ population, indicating low neutralization activity. This figure demonstrates the ability to successfully implement neutralization assays inside droplets for HIV-1 pseudovirus assays using NGS analysis of sorted cell libraries.

FIG. 32. An example assay arrangement for the detection of antibody binding to a membrane-bound target on a target cell. Inside compartments, the HEK293FT cell secretes a soluble antibody (green) that binds to a membrane protein (yellow). After antibody binds inside droplets or other compartments, with hundreds to trillions or more cells compartmentalized inside the compartments, the cells are recovered in bulk and stained with another detection antibody (blue) to identify the surface bound binding event. In some embodiments the detection antibody is labeled with a fluorophore (here APC is shown). The detection antibody could bind to the constant region or Fc portion, or to the variable region or to a peptide fusion on the secreted antibody itself. The detection antibody could utilize a peptide tag for MACS, or a DNA barcode, or other means of detection known to those skilled in the art. A HEK293FT cell is shown, but in other embodiments the cell could represent any mammalian, bacterial, insect, fungal, or other cell type.

FIG. 33. An example assay arrangement for the detection of Fc effector engagement to a membrane surface protein (e.g., membrane-bound target) on a target cell. Inside compartments, the HEK293FT cell secretes a soluble antibody (green) that binds to a membrane protein (yellow). After antibody binds inside droplets or other compartments, with hundreds to trillions or more cells compartmentalized inside compartments, the cells are recovered in bulk and stained with Fc engagement proteins (purple), shown here as FcgRIIa/FcgRIIb/FcgRIII, but that could represent any Fc engagement protein including FcgR, FcaR, FcmR, and FceR protein variants. The Fc engagement proteins are shown in a dimeric format but could be in a monomer format or any other format containing Fc engagement proteins. The detection agent is shown here as a fluorophore (PE) but could be any detection agent known to those skilled in the art, including DNA barcodes, peptide tags, fluorescent moieties, and other detection agents. A HEK293FT cell is shown, but in other embodiments the cell could represent any mammalian, bacterial, insect, fungal, or other cell type.

FIG. 34. A method of sorting for antibodies with high Fc protein engagement activity after recovering cells from compartments. The x-axis detects the amount of antibody bound to single cells (APC channel), and the y-axis detects Fc effector protein engagement (PE channel). Antibodies can thus be fractionated into different populations based on their potency of Fc protein engagement, compared to the amount of antibody bound to the cell surface. In some embodiments, Fe effector engagement can be analyzed alone, or in combination with other single-cell detection targets.

FIG. 35. An example assay arrangement for the detection of complement engagement to a membrane-bound target on a target cell. Inside compartments, the HEK293FT cell secretes a soluble antibody (green) that binds to a membrane protein (yellow). After polypeptide binds inside droplets or other compartments, with hundreds to trillions or more cells compartmentalized inside the compartments, the cells are recovered in bulk and stained with complement engagement proteins (yellow), shown here as C1q, but that could represent any complement engagement protein including a complex of C1q with C1s and/or C1r. The complement engagement proteins are shown in a hexameric format but could be in a monomer domain format or any other format containing complement engagement proteins. The detection agent is shown here as an antibody binding to the complement protein conjugated to a fluorophore (PE) but could be any detection agent known to those skilled in the art, including DNA barcodes, peptide tags, fluorescent moieties, and other detection agents. The complement protein itself could be fused to a detection agent, rather than using another antibody that specifically recognizes C1q or other complement proteins. In some embodiments, complement protein engagement can be analyzed alone, or in combination with other single-cell detection targets. A HEK293FT cell is shown, but in other embodiments the cell could represent any mammalian, bacterial, fungal, or other cell type.

FIG. 36. A method for implementation of high-throughput polypeptide-target interaction screening, shown here for antibody:antigen interaction library screening. In the present example, antibody and antigen libraries expressed in mammalian cells are captured inside single-cell droplets, for truly high-throughput screening data for paired antibody:antigen interactions. This unique approach can use CRISPR-based precision cloning of antibody libraries and antigen libraries for large-scale experimental pairwise polypeptide-target interaction data. Ag-antigen, AgBC-antigen barcode, AbBC-antibody barcode, HA-L-left homology arm, HA-R-right homology arm

FIG. 37. An illustration of a study of binding interactions between secreted antibodies and membrane-bound antigens (e.g., membrane-bound targets). Briefly, genes encoding surface HIV-1 Env BG505 SOSIP and secreted antibodies (1-20, negative control that does not bind to HIV-1 Env, and VRCO1, a positive control that binds to HIV-1 Env), were expressed by HEK293 cells. Cells were compartmentalized within droplets and incubated for 24 hrs. Next, cells were recovered and stained with anti-Kappa-FITC, then fixed. Anti-Kappa-FITC binding was measured for cells expressing mCherry via FACS.

FIG. 38. Graphs demonstrating a validation of appropriate binding interactions between control membrane-bound target:secreted polypeptide pairs. Cells expressing a secreted test polypeptide (IgG1) and a membrane-bound target (HIV-1 Env) were analyzed following the procedure shown in FIG. 38. Cells expressing an HIV-1 Env binding antibody, VRCO1, had over 5 times more anti-Kappa binding than cells expressing the HIV-1 Env non-binding antibody, 1-20.

FIG. 39. Diagrams of example plasmids encoding secreted test polypeptides (here in the form of IgG), and a membrane-bound target (here in the form of HIV-1 Env antigen). These plasmids are compatible with Cas9-based gene integration into cell genomes. In this example, the same plasmid encodes for both a secreted test polypeptide, and also a membrane-bound target.

FIG. 40. A diagram of an ELISA-based assay and a graph illustrating ELISA-based validation of appropriate antigen binding for control antibodies. ELISA assays were used to validate the binding of control antibodies secreted by cells and secreted test polypeptides. Plates were coated with BG505 SOSIP HIV-1 Env. Cell-secreted test polypeptide antibodies were used as primary antibodies against the BG505 SOSIP HIV-1 Env coated onto the plates. An anti-human IgG Fc-HRP conjugated antibody was used as the secondary stain. All positive control (binding) antibodies showed recognition of HIV-1 Env (VRCO1, VRC34), whereas negative controls (including a non-HIV-1 binding antibody, 72A1) showed no absorbance. These data demonstrated the capacity of cells to secrete test polypeptides with appropriate activity.

FIG. 41. A diagram of an ELISA-based assay and a graph illustrating ELISA-based validation of appropriate Fc protein recruitment for control antibodies. ELISA assays were used to validate the binding of Fc gamma receptors to test polypeptides in the form of IgG1. Cell supernatants containing secreted test polypeptides (IgG1) were used to coat ELISA plates, and biotinylated FcgR dimers were used as a primary stain. An anti-biotin HRP conjugated antibody was used as the secondary stain. All antibodies showed binding to multiple FcgR proteins, including variants of FcgRIIa and FcgRIIIa. These data demonstrated the capacity of cells to secrete test polypeptides with appropriate capacity to bind Fc effector proteins.

FIG. 42A-E. BG505.SOSIP native trimer surface plasmid expression in two cell lines BG505.SOSIP plasmid expression was verified in native trimer surface expression in two cell lines. Transfection was conducted with Lipo3000 on HEK-239T and TZM-GFP. FIG. 42A-B: fluorescence microscopy and flow cytometry images, respectively, of HEK-293T cells expressing the YFP marker on the BG505.SOSIP expression plasmid. FIG. 42C-D: fluorescence microscopy and flow cytometry images, respectively, of TZM-GFP cells expressing the YFP marker on the BG505.SOSIP expression plasmidFigure 42E. A graph demonstrating percentage of cells expressing YFP in HEK293T and TZM=GFP cells. HEK-293T: ˜25% YFP+/TZM-GFP: ˜9% YFP+. Cell counts were obtained via ImageJ processing & flow cytometry; YFP fluorescence was compensated with turboYFP. >100K cells were analyzed per flow test. These data demonstrate successful protein expression from plasmids encoding a membrane-bound target antigen after transfection of cell lines.

FIG. 43. Validated interactions between a secreted polypeptide (an IgG antibody) and its membrane-bound target (an HIV-1 BG505-SOSIP trimer). Cell-based binding assays were used to validate the binding of Fc gamma receptors to test polypeptides in the form of IgG1 bound to cell-displayed trimer antigens in well plates. This experiment evaluated the ability of cells to secrete a polypeptide (IgG1 antibody) that would bind appropriately to a membrane-bound target (a BG505.SOSIP trimer antigen). The markedly higher 450 nm absorption signals from wells containing cells secreting antibody and expressing BG505.SOSIP trimer concurrently, compared to lower signals for WT cells or cells expressing antibody alone (no BG505.SOSIP trimer) demonstrated that cells can both express a membrane-bound target (BG505.SOSIP trimer) and concurrently capture secreted polypeptides (IgG1) in a format that enables cell isolation and high-throughput secreted polypeptide binding assays for recognition of desired membrane-bound targets (cell surface targets).

FIG. 44. Validated Fc effector probe recruitment (a detection agent bound to an Fc effector) by a secreted polypeptide (an IgG antibody) bound to its membrane-bound target (an HIV-1 BG505-SOSIP trimer) This experiment validated the ability of secreted control polypeptides binding to membrane-bound targets and also recruit Fc effector proteins The markedly higher 450 nm absorption signals from wells containing cells secreting antibody and expressing BG505.SOSIP trimer concurrently, compared to lower signals for WT cells or cells expressing a non-HIV binding antibody demonstrated that cells can both express a membrane-bound target (BG505.SOSIP trimer) and concurrently capture secreted polypeptides (IgG1) that then recruit FcgR proteins. These data enable cell isolation into a compartment and high-throughput secreted polypeptide binding assays for Fc effector protein recruitment against membrane-bound targets.

FIG. 45. Secreted polypeptide (IgG1) and membrane-bound target (HIV-1 Env) expression inside droplet compartments, with staining for Fc effector protein recruitment after recovery of cells from droplets. This experiment validated the ability of cells inside a compartment to secrete a polypeptide (IgG1) specific to a membrane-bound target (HIV-1 Env), and detect a positive interaction using Fc effector protein detection agents (recombinant soluble dimeric FcgRIIb, FcgRIIa, and FcgRIIIa). The presence of FcgR binding is detected on cells when they are expressed concurrently with HIV-1 Env trimer and IgG1 specific to HIV-1 Env (either VRCO1 or VRC07-523LS). These data demonstrate cell isolation into a compartment and high-throughput secreted polypeptide binding assays for Fc effector protein recruitment against membrane-bound targets.

### DETAILED DESCRIPTION

The present invention is described herein using several definitions, as set forth below and throughout the application.

Unless otherwise specified or indicated by context, the terms “a,” “an,” and “the” mean “one or more.” For example, “an inhibitor of tumor cell aggregation” should be interpreted to mean “one or more inhibitors of tumor cell aggregation.”

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus <10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, the terms “protein,” “peptide,” and “polypeptide” are used interchangeably.

Disclosed herein are methods, compositions, systems, and kits for the functional screening of soluble protein libraries in a rapid, high throughput, and cost-effective manner.

By way of example and as described herein, exemplary cell lines were generated that are permissible to viral infection and concurrent antibody secretion to analyze the viral neutralization features of the produced antibodies.

In some embodiments, a cell line is provided or engineered that is susceptible to SARS-CoV-2 infection, and that also secretes antibodies, or antigen binding fragments thereof. In some embodiments, the ability of the secreted antibodies to neutralize, prevent, or reduce viral infection (SARS-coV-2 infection) of the antibody secreting cell is analyzed.

In some embodiments, a cell line is provided or engineered that is susceptible to HIV infection, and that also secretes anti-HIV antibodies. In some embodiments, the ability of the secreted antibodies to neutralize, prevent, or reduce viral infection (HIV infection) of the antibody secreting cell is analyzed.

In some embodiments, a cell line that is already permissible to viral infection (e.g., Raji-DC-SIGN with yellow fever virus recombinant viral particles) is used.

In some embodiments, antibody expression may be engineered into a mammalian cell line that is natively capable of virus infection. In some embodiments, the cell line is engineered to express at least one component of viral entry (e.g., a heterologous cell surface molecule or membrane-bound target).

In some embodiments, a secreted polypeptide (such as a potential ligand or a potential ligand-receptor antagonist or agonist) is expressed in a cell line that has been generated for the purpose of analysis of ligand-receptor agonism or antagonism (e.g., for the PD-1 surface receptor).

### Methods of Screening

Alternative approaches for functional analysis of secreted polypeptide molecules currently known in the prior art (e.g., the screening performed by the AbCheck company). Other systems are shown. For example, Weikang et al., discloses multi-cell droplet compartmentalization, along with sorting of a sensor cell and the polypeptide-secreting cell (see e.g., Lin W N, Tay M Z, Wong J X E, Lee C Y, Fong S W, Wang C I, Ng L F P, Renia L, Chen C H, Cheow L F. Rapid microfluidic platform for screening and enrichment of cells secreting virus neutralizing antibodies. Lab Chip. 2022 Jun. 28; 22(13):2578-2589). These dual-cell approaches within a single droplet generally have much lower throughput compared to single-cell droplet systems. Additionally, these platforms present technical complexity to be able to sort and select for droplets containing multiple cells. In contrast, our approach enables the recovery of the polypeptide-secreting cell linked with a selection marker associated with the activity of the antibody, enabling facile selection of desired functionality.

Some alternative published approaches may screen membrane-associated polypeptides for the interruption of receptor binding as a proxy signal for polypeptide activity, including virus neutralization (e.g., blocking ACE2 binding to the SARS-CoV-2 fusion protein) (see e.g., Shiakolas, A. R. et al., Efficient discovery of SARS-CoV-2-neutralizing antibodies via B cell receptor sequencing and ligand blocking. Nat Biotechnol (2022). doi:10.1038/s41587-022-01232-2).

However, a screen for receptor binding inhibition does not screen for neutralization directly, and there are many antibodies that will be missed from a selection round when screening for the interruption of receptor binding. Furthermore, screening for ligand blocking is unable to efficiently select for agonist antibodies. In contrast, herein we demonstrate the ability to screen for neutralization and for agonist antibodies directly using the current technology.

As disclosed herein, it is highly advantageous to implement high-throughput assays using single cells, rather than multiple cells inside droplets, for enhanced throughput and simplicity for the assay. Additionally, it is advantageous to be able to sort single cells, rather than sorting droplets, because single cells can be sorted using a broader range of cellular equipment (e.g., many different types of FACS machines available from multiple different vendors), whereas droplet sorting often requires specialized and/or custom equipment to implement.

Some alternative approaches perform binding assay screens for polypeptide secreted cells inside droplets (see e.g., Gerard, A. et al., High-throughput single-cell activity-based screening and sequencing of antibodies using droplet microfluidics. Nature Biotechnology 1-7 (2020). doi:10.1038/s41587-020-0466-7), for example, plasma cells secreting antibody. However, these technologies require droplet-based sorting which is complicated and inefficient, and the use of plasma cells prevents most neutralization assays and selections for agonist or antagonist molecules against membrane proteins like GPCRs. In contrast, our approach allows sorting of cells directly using standard FACS equipment, and is flexible for a broad range of membrane protein-based selections and viral neutralization assays that show major advantages compared with the technologies described in the prior art.

The procedures described here could be implemented with sorting cell droplets on a microfluidic chip as one variant of the procedure (e.g., sorting droplets before breaking the emulsions and collecting the cells), if desired.

In one aspect of the current disclosure, screening methods are provided. In some embodiments, the screening methods comprise: (a) detecting the presence and/or level of expression of a reporter molecule in a single, isolated, genetically engineered cell, wherein the cell presents a membrane-bound target; and wherein the cell is engineered to: (i) secrete a secreted polypeptide; and (ii) express a reporter molecule if the test polypeptide activates the membrane-bound target.

As used herein, “presents a membrane-bound target” or “presenting the membrane-bound target” refers to the cell of interest having the membrane-bound target localized to the cell surface. Localization of the membrane-bound target may depend on the unique molecular properties of the membrane-bound target itself. In addition, the localization of the membrane-bound target may be required for function of the protein. In some embodiments, the membrane-bound target comprises an integral membrane protein. In some embodiments, the membrane-bound target is localized to the cell surface by a glycosylphosphatidylinositol (GPI) moiety. In some embodiments, the membrane-bound target is capable of transducing a signal across the cell membrane into the cell. In other embodiments, the membrane-bound target is present to allow entry of a test reagent, which may, e.g., comprise a reporter molecule. In some embodiments, the membrane-bound target is expressed by the cell and is then localized to the cell membrane. In other embodiments, the membrane-bound target is delivered to the cell by means known in the art, e.g., exosomes, microvesicles, liposomes, etc.

As used herein, “associated with a cell” refers to any entity within, linked to, or bound to the cell. For example, a “reporter molecule associated with a cell” refers to a reporter molecule, or a signal from the reporter molecule, inside the cell, and/or outside the cell, but operatively linked to the cell (e.g., wherein the reporter molecule is able to make the cell detectable).

As used herein, “membrane-bound target,” refers to any cell surface-associated or membrane-bound molecule. In some embodiments, the membrane-bound target comprises a protein, e.g., for a ligand that is capable of transducing a signal inside the cell upon receptor ligation. Thus, in some embodiments, a membrane-bound target comprises a cell surface receptor. In some embodiments, a membrane-bound target comprises another molecule that is not a cell surface receptor. By way of example, but not by way of limitation, the membrane-bound target may comprise a polypeptide sequence, a polypeptide sequence derived from an antibody or fragment thereof, such as from an scFv, chimeric antigen receptor (CAR), antigen binding fragment of heavy chain (VHH), nanobody, a chemical moiety, a polymer, an oligomer, a nucleic acid, a peptide sequence, a peptide sequence, or a fusion protein. Membrane-bound targets comprising polypeptide sequences may comprise post-translational modifications, non-natural amino acids, or other modifications. Membrane-bound targets may be expressed and presented by the cell on the cell surface, or the membrane-bound targets may be localized to the cell surface by chemical, enzymatic, or other synthetic means.

As used herein, a “detection agent” refers to an agent or probe used for the detection of the membrane-bound target, secreted polypeptide, and/or the complex formed by the binding, hybridization, or linking of the membrane-bound target with the secreted polypeptide. By way of example, but not by way of limitation, detection agents include nucleic acid probes or primers (e.g., which can hybridize to a nucleic acid sequence, or amplify a nucleic acid sequence linked to a membrane-bound target, or a secreted protein), an antibody Fc effector protein, a complement protein, a ligand, a chemical, a nucleic acid sequence, an antibody, a fragment of an antibody, or a polypeptide that is capable of binding the secreted polypeptide or the membrane-bound target. In embodiments, the detection agent includes a reporter molecule.

As used herein, a “reporter molecule” refers to a molecule capable of generating a detectable signal and indicating a particular molecular state or condition of the cell. For example, in some embodiments, cell lines are engineered to provide a signal (e.g., express a reporter molecule) in response to a receptor agonist or antagonist. Therefore, the reporter molecule indicates the state of the cell, i.e., that the receptor of interested has been bound and/or activated or has been prevented from being bound and/or activated. In some embodiments, a reporter is detected within a cell, indicating that the cell has been infected. By way of example, but not by way of limitation, reporter molecules include nucleic acid barcodes, dyes, peptide tags, fluorescent moieties, fluorescent proteins, enzymes, chemicals, and other detection agents. The detection agent may be a single entity that binds the membrane-bound target or secreted polypeptide, such as a fluorescently labeled antibody, or may be modular entity, such as a primary antibody that binds the membrane-bound target or secreted polypeptide, and a fluorescently labeled secondary antibody that binds the primary antibody.

As used herein, “detecting” refers to acquiring information provided by one or more reporters, e.g., such as a reporter in the cell. Accordingly, in some embodiments, detecting may be performed by an automated apparatus, e.g., a flow cytometer, fluorometer, luminometer, microscope, digital camera, plate reader, etc. or by the human eye. In other embodiments, detecting is performed using a technique related to the sequencing of nucleic acids, e.g., Sanger sequencing, next generation sequencing (NGS), single-cell RNA sequencing (scRNA-seq) etc., wherein the reporter comprises a barcode, or particular nucleic acid sequence.

Reporter molecules are well known in the art. By way of example only, and not be way of limitation, exemplary fluorescent proteins include, but are not limited to the molecules provided below, and functional variants thereof:

| Green fluorescent protein (GFP) , which has the sequence: | |
|---|---|
| (SEQ ID NO: 1) | |
| MSKGEELFTG VVPILVELDG DVNGHKESVS GEGEGDATYG KLTLKFICTT GKLPVPWPTL | 60 |
| VTTFSYGVQC FSRYPDHMKQ HDFFKSAMPE GYVQERTIFF KDDGNYKTRA EVKFEGDTLV | 120 |
| NRIELKGIDF KEDGNILGHK LEYNYNSHNV YIMADKQKNG IKVNFKIRHN IEDGSVQLAD | 180 |
| HYQQNTPIGD GPVLLPDNHY LSTQSALSKD PNEKRDHMVL LEFVTAAGIT HGMDELYK | 238 |
| Red fluorescent protein (RFP), which has the sequence: | |
| (SEQ ID NO: 2) | |
| MRGSHHHHHH GSAHGLTDDM TMHERMEGCV DGHKFVIEGN GNGNPFKGKQ FINLCVIEGG | 60 |
| PLPFSEDILS AAFXNRLFTE YPEGIVDYFK NSCPAGYTWH RSFRFEDGAV CICSADITVN | 120 |
| VRENCIYHES TFYGVNFPAD GPVMKKMTTN WEPSCEKIIP INSQKILKGD VSMYLLLKDG | 180 |
| GRYRCQFDTI YKAKTEPKEM PDWHFIQHKL NREDRSDAKN QKWQLIEHAI ASRSALP | 237 |
| Yellow fluorescent protein (YFP), which has the sequence: | |
| (SEQ ID NO: 3) | |
| KGEELFTGVV PILVELDGDV NGHKFSVSGE GEGDATYGKL TLKFICTTGK LPVPWPTLVT | 60 |
| TFXLQCFARY PDHMKRHDFF KSAMPEGYVQ ERTIFFKDDG NYKTRAEVKF EGDTLVNRIE | 120 |
| LKGIDFKEDG NILGHKLEYN YNSHNVYIMA DKQKNGIKVN FKIRHNIEDG SVQLADHYQQ | 180 |
| NTPIGDGPVL LPDNHYLSYQ SALSKDPNEK RDHMVLLEFV TAAGI | 225 |
| Blue fluorescent protein (BFP), which has the sequence: | |
| (SEQ ID NO: 4) | |
| MSKGEELFTG VVPILVELDG DVNGHKESVS GEGEGDATYG KLTLKFICTT GKLPVPWPTL | 60 |
| VTTFXVQCFS RYPDHMKRHD FFKSAMPEGY VQERTIFFKD DGNYKTRAEV KFEGDTLVNR | 120 |
| IELKGIDFKE DGNILGHKLE YNFNSHNVYI MADKQKNGIK VNFKIRHNIE DGSVQLADHY | 180 |
| QQNTPIGDGP VLLPDNHYLS TQSALSKDPN EKRDHMVLLE FVTAAGITHG MDELYK | 236 |
| Cyan fluorescent protein (CFP), which has the sequence: | |
| (SEQ ID NO: 5) | |
| MVSKGEELFT GVVPILVELD GDVNGHKFSV SGEGEGDATY GKLTLKFICT TGKLPVPWPT | 60 |
| LVTTLXVQCF ARYPDHMKQH DFFKSAMPEG YVQERTIFFK DDGNYKTRAE VKFEGDTLVN | 120 |
| RIELKGIDEK EDGNILGHKL EYNAISDNVY ITADKQKNGI KANFKIRHNI EDGSVQLADH | 180 |
| YQQNTPIGDG PVLLPDNHYL STQSALSKDP NEKRDHMVLL EFVTAAGITL GMDELYK | 237 |
| Or | |
| (SEQ ID NO: 6) | |
| MVSKGEELFT GVVPILVELD GDVNGHKFSV SGEGEGDATY GKLTLKFICT TGKLPVPWPT | 60 |
| LVTTLXVQCF SRYPDHMKQH DFFKSAMPEG YVQERTIFFK DDGNYKTRAE VKFEGDTLVN | 120 |
| RIELKGIDFK EDGNILGHKL EYNYISHNVY ITADKQKNGI KANFKIRHNI EDGSVQLADH | 180 |
| YQQNTPIGDG PVLLPDNHYL STQSALSKDP NEKRDHMVLL EFVTAAGITL GMDELYK | 237 |
| mCherry, which has the sequence: | |
| (SEQ ID NO: 7) | |
| MVSKGEEDNM AIIKEFMRFK VHMEGSVNGH EFEIEGEGEG RPYEGTQTAK LKVTKGGPLP | 60 |
| FAWDILSPQF MYGSKAYVKH PADIPDYLKL SFPEGFKWER VMNFEDGGVV TVTQDSSLQD | 120 |
| GEFIYKVKLR GTNFPSDGPV MQKKTMGWEA SSERMYPEDG ALKGEIKQRL KLKDGGHYDA | 180 |
| EVKTTYKAKK PVQLPGAYNV NIKLDITSHN EDYTIVEQYE RAEGRHSTGG MDELYK | 236 |
| Exemplary luminescent proteins include, but are not limited to: | |
| Renilla luciferase, which has the sequence: | |
| (SEQ ID NO: 8) | |
| MTSKVYDPEL RKRMITGPQW WARCKQMNVL DSFINYYDSE KHAENAVIEL HGNAASSYLW | 60 |
| RHVVPHVEPV ARCIIPDLIG MGKSGKSGNG SYRLLDHYKY LTEWFKHLNL PKKIIFVGHD | 120 |
| WGACLAFHYC YEHQDRIKAV VHAESVVDVI ESWDEWPDIE EDIALIKSEE GEKMVLENNE | 180 |
| FVETMLPSKI MRKLEPEEFA AYLEPFKEKG EVRRPTLSWP REIPLVKGGK PDVVEIVRNY | 240 |
| NAYLRASHDL PKMFIESDPG FFSNAIVEGA KKFPNTEFVK VKGLHESQED APDEMGNYIK | 300 |
| SFVERVLKNE Q | 311 |
| Firefly (Photinus pyralis) luciferase, which has the sequence: | |
| (SEQ ID NO: 9) | |
| MEDAKNIKKG PAPFYPLEDG TAGEQLHKAM KRYALVPGTI AFTDAHIEVN ITYAEYFEMS | 60 |
| VRLAEAMKRY GLNTNHRIVV CSENSLQFFM PVLGALFIGV AVAPANDIYN ERELLNSMNI | 120 |
| SQPTVVFVSK KGLQKILNVQ KKLPIIQKII IMDSKTDYQG FQSMYTFVTS HLPPGENEYD | 180 |
| FVPESFDRDK TIALIMNSSG STGSPKGVAL PHRTACVRES HARDPIFGNQ IIPDTAILSV | 240 |
| VPFHHGFGMF TTLGYLICGF RVVLMYRFEE ELFLRSLQDY KIQSALLVPT LFSFFAKSTL | 300 |
| IDKYDLSNLH EIASGGAPLS KEVGEAVAKR FHLPGIRQGY GLTETTSAIL ITPEGDDKPG | 360 |
| AVGKVVPFFE AKVVDLDTGK TLGVNQRGEL CVRGPMIMSG YVNDPEATNA LIDKDGWLHS | 420 |
| GDIAYWDEDE HFFIVDRLKS LIKYKGCQVA PAELESILLQ HPNIFDAGVA GLPGDDAGEL | 480 |
| PAAVVVLEHG KTMTEKEIVD YVASQVTTAK KLRGGVVEVD EVPKGLTGKL DARKIREILI | 540 |
| KAKKGGKSKL | 550 |

As used herein, “expression” refers to either the transcription of a nucleic acid comprising DNA into RNA or the translation of an RNA into a protein or polypeptide, or both the transcription of DNA into RNA and translation of said RNA into a protein or polypeptide.

The methods disclosed herein utilize an efficient single-cell platform that allows for rapid and high-throughput testing of candidate molecules. Thus, as used herein “single, isolated cell” refers to a cell that is physically separated from other cells in a reaction vessel, e.g., a multi-well plate, a microchip, a microfluidics chip, a Nanopen™, and the like.

The methods, compositions, systems, and kits of the instant disclosure utilize “genetically engineered cells.” As used herein, “genetically engineered,” or grammatical variations thereof, refers to the cell possessing one or more genetic modifications made by the hand of man. Such modifications comprise, for example, expression of an introduced or exogenous nucleic acid. Methods of introducing exogenous nucleic acids are known in the art including, but not limited to, transfection, lipofection, viral transduction, e.g., retroviral, lentiviral, or adenoviral transduction. In some embodiments, genetically engineered cells comprise nucleic acids that are integrated into the genome of the cell, while in other embodiments, genetically engineered cells comprise nucleic acids that are contained in episomes.

In some embodiments, genetically engineered cells comprise nucleic acids which encode genes of interest operably controlled by one or more promoters or one or more enhancer sequences. In some embodiments, the promoters may have constitutive activity, i.e., the promoters continuously direct transcription of the nucleic acid under its control. Exemplary constitutive promoters include but are not limited to the cytomegalovirus (CMV) promoter and elongation factor 1α (EF1α) promoter. In other embodiments, the one or more promoters are inducible, meaning that they respond to addition of another molecule. Exemplary inducible promoters include tetracycline inducible promoters, cumate inducible promoters, and estrogen receptor-based tamoxifen inducible promoters. In some embodiments, promoters are “strong” promoters, with relatively high levels of expression of the downstream sequence. In some embodiments, promoters are “weak” promoters, with relatively low levels of expression of the downstream sequence. By way of example but not by way of limitation, the mammalian CMV promoter is generally considered to be a strong promoter by those skilled in the art.

In some embodiments, the methods of the present disclosure use a single cell as source of expression of both a protein of interest, such as a “membrane-bound target,” and a potential ligand of interest, such as a “secreted polypeptide.” In some embodiments, the genetically modified cells express a reporter in response to a successful interaction between the two proteins of interest (e.g., binding of the test peptide to the membrane-bound target and subsequent activity), such as, in some examples, downstream signaling by the receptor of interest due to activation by the secreted polypeptide. In some embodiments, each cell to be screened is engineered to express (1) a different potential ligand for the receptor of interest, (2) the receptor itself, and (3) the reporter molecule that indicates ligation of the receptor. Thus, screening of many such cells reveals a plurality of ligands for the receptor.

In some embodiments, the secreted polypeptide may have agonist or antagonist activity with respect to a membrane-bound target, such as a receptor. Exemplary non-limiting agonists or antagonists include molecules such as an antibody, or an antigen binding fragment thereof, e.g., a single-chain variable fragment (scFv), nanobody, or Fab fragment.

As used herein, “single-chain variable fragment (scFv)” refers to single immunoglobulin heavy and single immunoglobulin light chain fused by a linker. As used herein, a “nanobody” refers to a protein comprising a single monomeric variable antibody domain. “Fab” fragment refers to the antigen-binding region of an antibody.

By way of example, but not by way of limitation, the secreted polypeptide may include a polypeptide sequence derived from an antibody or fragment thereof, such as from an scFv, chimeric antigen receptor (CAR), antigen binding fragment of heavy chain (VHH), or a nanobody. By way of example, but not by way of limitation, the secreted polypeptide may include a chemical moiety, a polymer, an oligomer, a nucleic acid, a peptide sequence, or a fusion protein.

In some embodiments, a ligand for a membrane-bound target is known and the secreted polypeptide has a structure or sequence based on that of the known ligand.

In some embodiments the screening methods comprise: (a) detecting the presence and/or level of expression of a reporter molecule in a single, isolated, genetically engineered cell, wherein the cell presents a membrane-bound target; and wherein the cell is engineered to: (i) secrete a secreted polypeptide; and (ii) express a reporter molecule if the test polypeptide does not activate the membrane-bound target. Thus, in such embodiments, prevention of activation of the membrane-bound target is revealed by the expression of the reporter molecule.

In some embodiments of the screening methods, the screening methods comprise: (a) contacting a single, isolated, genetically engineered cell with a test reagent, wherein the cell presents a membrane-bound target, and wherein the cell is engineered to: (i) secrete a secreted polypeptide; and (ii) express a reporter molecule if one of the test polypeptide or the test reagent activates the membrane-bound target; (b) detecting the presence and/or level of expression of the reporter molecule.

In some embodiments, the test reagent includes a ligand of a membrane-bound target, and the secreted polypeptide is a potential agonist or antagonist of the membrane-bound target. In other embodiments, the test reagent includes an antagonist or an agonist of the membrane-bound target, and the secreted polypeptide includes a potential ligand of the membrane-bound target.

In some embodiments, the screening methods comprise: (a) contacting a single, isolated, genetically engineered cell with a test reagent comprising a reporter molecule, wherein the cell presents a membrane-bound target; wherein the test reagent is capable of binding the membrane-bound target presented by the cell, forming a reagent-protein complex, and wherein the test reagent gains entry into the cell when the reagent-protein complex is formed; wherein the cell is engineered to: (i) secrete a secreted polypeptide; (b) detecting the presence and/or level of expression of the reporter molecule in the cells.

In some embodiments, the test reagent includes an infectious agent, or is derived from an infectious agent. In some embodiments, the test reagent includes a virus, or is derived from a virus. Exemplary, non-limiting viruses include, for example, Coronavirus A, B, C, D, flaviviruses, lentiviruses, influenza A, B, C, or D viruses, Epstein-Barr virus, cytomegalovirus, respiratory syncytial virus, Ebola virus, Marburg virus, Dengue virus. In some embodiments, the test reagent is, or is derived from, human immunodeficiency virus (HIV), yellow fever virus, severe acute respiratory syndrome coronavirus-2 (SARS-CoV-2), Epstein-Barr virus, cytomegalovirus, respiratory syncytial virus, Ebola virus, Marburg virus, or Dengue virus. In some embodiments, the test reagent includes a pseudovirus. As used herein, “pseudovirus” refers to a replication incompetent virus, or viral-like particle, often based on retroviruses, lentiviruses, e.g., HIV, or vesicular stomatitis virus, which additionally comprise a key viral factor from another virus, e.g., SARS-CoV-2 surface glycoprotein (spike protein). Thus, the risk of infection to researchers using the pseudovirus is mitigated, compared to the use of wild type virus, while being useful as a tool for the discovery of novel neutralization agents against the wild type virus, as in the methods, compositions, and kits disclosed herein. In some embodiments, the virus is capable of infecting a mammal, a fish, an avian, a plant, an insect, a yeast, or a bacterium.

In some embodiments, the test reagent comprises a reporter molecule. Therefore, once the test reagent comprising the reporter molecule binds the membrane-bound target and gains entry into the cell, the cell comprises the reporter molecule.

In some embodiments, the membrane-bound target includes a receptor that is required for complexing with the test reagent and catalyzing entry of the test reagent into the cell. In some embodiments the membrane-bound target includes a receptor for a ligand that is capable of transducing a signal inside the cell upon receptor ligation

Exemplary membrane-bound targets include, but are not limited to:

| Human angiotensin converting enzyme 2 (hACE-2), which has the | |
|---|---|
| amino acid sequence: | |
| (SEQ ID NO: 10) | |
| MSSSSWLLLS LVAVTAAQST IEEQAKTELD KFNHEAEDLF YQSSLASWNY NTNITEENVQ | 60 |
| NMNNAGDKWS AFLKEQSTLA QMYPLQEIQN LTVKLQLQAL QQNGSSVLSE DKSKRINTIL | 120 |
| NTMSTIYSTG KVCNPDNPQE CLLLEPGLNE IMANSLDYNE RLWAWESWRS EVGKQLRPLY | 180 |
| EEYVVLKNEM ARANHYEDYG DYWRGDYEVN GVDGYDYSRG QLIEDVEHTF EEIKPLYEHL | 240 |
| HAYVRAKLMN AYPSYISPIG CLPAHLLGDM WGREWTNLYS LTVPFGQKPN IDVTDAMVDQ | 300 |
| AWDAQRIFKE AEKFFVSVGL PNMTQGFWEN SMLTDPGNVQ KAVCHPTAWD LGKGDERILM | 360 |
| CTKVTMDDEL TAHHEMGHIQ YDMAYAAQPF LLRNGANEGF HEAVGEIMSL SAATPKHLKS | 420 |
| IGLLSPDFQE DNETEINELL KQALTIVGTL PFTYMLEKWR WMVFKGEIPK DQWMKKWWEM | 480 |
| KREIVGVVEP VPHDETYCDP ASLFHVSNDY SFIRYYTRTL YQFQFQEALC QAAKHEGPLH | 540 |
| KCDISNSTEA GQKLENMLRL GKSEPWTLAL ENVVGAKNMN VRPLLNYFEP LFTWLKDQNK | 600 |
| NSFVGWSTDW SPYADQSIKV RISLKSALGD KAYEWNDNEM YLERSSVAYA MRQYFLKVKN | 660 |
| QMILFGEEDV RVANLKPRIS ENFFVTAPKN VSDIIPRTEV EKAIRMSRSR INDAFRLNDN | 720 |
| SLEFLGIQPT LGPPNQPPVS IWLIVFGVVM GVIVVGIVIL IFTGIRDRKK KNKARSGENP | 780 |
| YASIDISKGE NNPGFQNTDD VQTSF | 805 |
| Human programmed cell death 1 protein (PD-1), which has the sequence: | |
| (SEQ ID NO: 11) | |
| MEDAKNIKKG PAPFYPLEDG TAGEQLHKAM KRYALVPGTI AFTDAHIEVN ITYAEYFEMS | 60 |
| VRLAEAMKRY GLNTNHRIVV CSENSLQFFM PVLGALFIGV AVAPANDIYN ERELLNSMNI | 120 |
| SQPTVVFVSK KGLQKILNVQ KKLPIIQKII IMDSKTDYQG FQSMYTFVTS HLPPGENEYD | 180 |
| FVPESEDRDK TIALIMNSSG STGSPKGVAL PHRTACVRES HARDPIFGNQ IIPDTAILSV | 240 |
| VPFHHGFGMF TTLGYLICGF RVVLMYRFEE ELFLRSLQDY KIQSALLVPT LESFFAKSTL | 300 |
| IDKYDLSNLH EIASGGAPLS KEVGEAVAKR FHLPGIRQGY GLTETTSAIL ITPEGDDKPG | 360 |
| AVGKVVPFFE AKVVDLDTGK TLGVNQRGEL CVRGPMIMSG YVNDPEATNA LIDKDGWLHS | 420 |
| GDIAYWDEDE HFFIVDRLKS LIKYKGCQVA PAELESILLQ HPNIFDAGVA GLPGDDAGEL | 480 |
| PAAVVVLEHG KTMTEKEIVD YVASQVTTAK KLRGGVVFVD EVPKGLTGKL DARKIREILI | 540 |
| KAKKGGKSKL | 550 |
| Human cytotoxic T lymphocyte protein 4 (CTLA-4), which has the | |
| sequence: | |
| (SEQ ID NO: 12) | |
| MACLGFQRHK AQLNLATRTW PCTLLFFLLF IPVECKAMHV AQPAVVLASS RGIASFVCEY | 60 |
| ASPGKATEVR VTVLRQADSQ VTEVCAATYM MGNELTELDD SICTGTSSGN QVNLTIQGLR | 120 |
| AMDTGLYICK VELMYPPPYY LGIGNGTQIY VIDPEPCPDS DELLWILAAV SSGLFFYSEL | 180 |
| LTAVSLSKML KKRSPLTTGV YVKMPPTEPE CEKQFQPYFI PIN | 223 |
| Human 4-1BB, which has the sequence: | |
| (SEQ ID NO: 13) | |
| MGNSCYNIVA TLLLVLNFER TRSLQDPCSN CPAGTFCDNN RNQICSPCPP NSESSAGGQR | 60 |
| TCDICRQCKG VERTRKECSS TSNAECDCTP GFHCLGAGCS MCEQDCKQGQ ELTKKGCKDC | 120 |
| CFGTENDQKR GICRPWTNCS LDGKSVLVNG TKERDVVCGP SPADLSPGAS SVTPPAPARE | 180 |
| PGHSPQIISF FLALTSTALL FLLFFLTLRF SVVKRGRKKL LYIFKQPFMR PVQTTQEEDG | 240 |
| CSCRFPEEEE GGCEL | 255 |
| Human hepatitis A virus cellular receptor 2 (TIM-3), which has | |
| the sequence: | |
| (SEQ ID NO: 14) | |
| MFSHLPFDCV LLLLLLLLTR SSEVEYRAEV GQNAYLPCFY TPAAPGNLVP VCWGKGACPV | 60 |
| FECGNVVLRT DERDVNYWTS RYWLNGDERK GDVSLTIENV TLADSGIYCC RIQIPGIMND | 120 |
| EKFNLKLVIK PAKVTPAPTR QRDFTAAFPR MLTTRGHGPA ETQTLGSLPD INLTQISTLA | 180 |
| NELRDSRLAN DLRDSGATIR IGIYIGAGIC AGLALALIFG ALIFKWYSHS KEKIQNLSLI | 240 |
| SLANLPPSGL ANAVAEGIRS EENIYTIEEN VYEVEEPNEY YCYVSSRQQP SQPLGCREAM | 300 |
| P | 301 |
| Human lymphocyte activation gene 3 (LAG3), which has the sequence: | |
| (SEQ ID NO: 15) | |
| MWEAQFLGLL FLQPLWVAPV KPLQPGAEVP VVWAQEGAPA QLPCSPTIPL QDLSLLRRAG | 60 |
| VTWQHQPDSG PPAAAPGHPL APGPHPAAPS SWGPRPRRYT VLSVGPGGLR SGRLPLQPRV | 120 |
| QLDERGRQRG DESLWLRPAR RADAGEYRAA VHLRDRALSC RLRLRLGQAS MTASPPGSLR | 180 |
| ASDWVILNCS FSRPDRPASV HWERNRGQGR VPVRESPHHH LAESFLFLPQ VSPMDSGPWG | 240 |
| CILTYRDGEN VSIMYNLTVL GLEPPTPLTV YAGAGSRVGL PCRLPAGVGT RSELTAKWTP | 300 |
| PGGGPDLLVT GDNGDFTLRL EDVSQAQAGT YTCHIHLQEQ QLNATVTLAI ITVTPKSFGS | 360 |
| PGSLGKLLCE VTPVSGQERF VWSSLDTPSQ RSESGPWLEA QEAQLLSQPW QCQLYQGERL | 420 |
| LGAAVYFTEL SSPGAQRSGR APGALPAGHL LLFLILGVLS LLLLVTGAFG FHLWRRQWRP | 480 |
| RRESALEQGI HPPQAQSKIE ELEQEPEPEP EPEPEPEPEP EPEQL | 525 |

hACE-2 is required for SARS-CoV-2 entry into cells, while TMPRSS2 potentiates the entry of the virus into the cell. Thus, in some embodiments, cells of the instant disclosure may comprise both hACE-2 and TMPRSS2.

| Human transmembrane serine protease 2 (TMPRSS2), which has the sequence: | |
|---|---|
| (SEQ ID NO: 16) | |
| MALNSGSPPA IGPYYENHGY QPENPYPAQP TVVPTVYEVH PAQYYPSPVP QYAPRVLTQA | 60 |
| SNPVVCTQPK SPSGTVCTSK TKKALCITLT LGTFLVGAAL AAGLLWKEMG SKCSNSGIEC | 120 |
| DSSGTCINPS NWCDGVSHCP GGEDENRCVR LYGPNFILQV YSSQRKSWHP VCQDDWNENY | 180 |
| GRAACRDMGY KNNFYSSQGI VDDSGSTSEM KLNTSAGNVD IYKKLYHSDA CSSKAVVSLR | 240 |
| CIACGVNLNS SRQSRIVGGE SALPGAWPWQ VSLHVQNVHV CGGSIITPEW IVTAAHCVEK | 300 |
| PLNNPWHWTA FAGILRQSEM FYGAGYQVEK VISHPNYDSK TKNNDIALMK LQKPLTENDL | 360 |
| VKPVCLPNPG MMLQPEQLCW ISGWGATEEK GKTSEVLNAA KVLLIETQRC NSRYVYDNLI | 420 |
| TPAMICAGFL QGNVDSCQGD SGGPLVTSKN NIWWLIGDTS WGSGCAKAYR PGVYGNVMVF | 480 |
| TDWIYRQMRA DG | 492 |

Programmed cell death protein 1 (PD-1) is a transmembrane protein which contains immunoreceptor tyrosine-based inhibitory motifs (ITIMS) and an immunoreceptor tyrosine-based switch motif, which suggests that PD-1 negatively regulates T-cell receptor TCR signals. Agents which block PD-1 signaling, therefore, increase T cell effector function and have been used successfully to treat cancer.

Human cytotoxic T lymphocyte protein 4 (CTLA-4) is a transmembrane protein that binds to the co-stimulatory molecules CD80 and CD86 on antigen presenting cells (APCs) and transduces co-inhibitory signals to the T cell. Agents which block CTLA-4 signaling, therefore, increase T cell effector function and have been used successfully to treat cancer.

4-1BB (CD137, or TNFRSF9) is a membrane protein that acts to stimulate the effector function of T cells. Therefore, agents that modulate 4-1BB signaling may be useful for the treatment of human disease. For example, agents that stimulate 4-1BB may be useful to activate tumor infiltrating lymphocytes to destroy cancer cells, while agents that antagonize 4-1BB signaling may be useful to prevent autoimmunity or treat transplant-related symptoms in humans.

Human hepatitis A virus cellular receptor 2 (TIM-3) is a transmembrane protein that acts as an inhibitory molecule in T cells. Therefore, agents that reduce or block TIM-3 signaling may be useful in cancer immunotherapy.

Human lymphocyte activation gene 3 (LAG3) is a is a transmembrane protein that acts as an inhibitory molecule in T cells. Therefore, agents that reduce or block LAG3 signaling may be useful in cancer immunotherapy.

In some embodiments, the secreted polypeptide secreted by cells comprises any protein that may neutralize the virus or alter cell function to prevent viral infection. Exemplary secreted proteins may include interferon variants, griffithsin, peptides, receptor traps (e.g., soluble ACE2 variants for SARS-CoV-2, or soluble CD4 variants for HIV-1).

In some embodiments of the screening methods, the methods further comprise amplifying and/or sequencing the DNA encoding the secreted polypeptide. Thus, in some embodiments, cells that express the reporter molecule may be separated from those not expressing the reporter molecule by methods known in the art, e.g., fluorescence activated cell sorting (FACS), magnetic bead enrichment, and each group sequenced to produce libraries of sequences encoding secreted polypeptides associated with the expression, or lack of expression of the reporter in the given system.

In some embodiments, the reporter molecule comprises a nucleic acid sequence. In some embodiments, said nucleic acid sequence comprises a barcode sequence. As used herein, “barcode” or “barcode sequence” refers to a unique nucleotide sequence used to identify a particular condition, e.g., ligation or binding of a membrane-bound target. Barcode sequences suitably comprise sequences that are not found in the genome, transcriptome, exogenous expression vectors, etc. present in the cell in which the barcodes are expressed so as to be readily identifiable. The DNA barcode may be a DNA-barcoded moiety that is attached or otherwise associated with the membrane-bound target or the secreted peptide (e.g., a DNA barcoded polypeptide).

The present technology is not limited to a specific cell type or a specific cell line, and any suitable cell, including prokaryotic cells (e.g., bacterial), yeast, mammal, avian, fish, or plant cells may be used for both viral infection neutralization assays, and to test polypeptide-membrane-bound target activity (e.g., antibody, ligand, receptor, agonist, antagonist, etc.). Exemplary, non-limiting cell lines useful for the screening assays disclosed herein such as neutralization assays, include CHO, BHK, Cos-7 NSO, SP2/0, YB2/0, HEK293, HT-1080, Huh-7, PER.C6, and variants thereof, or others. In some embodiments, B cell lines may be used, for example Raji, ARH-77, MOPC-315, MOPC-21, or others. In some embodiments, cancer cell lines, or cell lines representative of, or characteristic of a particular organ, tissue, or disease state, or state of differentiation and development may be used. The cell type can vary across embodiments, as many different cell types can be used for polypeptide expression. A variety of cell lines and types are known to those skilled in the art.

The present technology is not limited to a specific type or class of polypeptide or membrane-bound target. Any suitable secreted polypeptide can be used, and in some embodiments, a secreted polypeptide may be combined with another entity to comprise the test reagent. For example, a secreted polypeptide may be combined with an oligonucleotide-labeled polypeptide⁷² or with a DNA-encoded chemical library⁷¹, or combined with other entities according to methods known to those skilled in the art. Similarly, the membrane-bound target may comprise a variety of different entities, including polypeptides, glycans, lipids, oligonucleotide-labeled polypeptide⁷², a DNA-encoded chemical library⁷¹, an endogenously expressed polypeptide, a fusion protein, or other types of membrane-bound targets that are known to those skilled in the art.

By way of example, in some embodiments, insect cells may be used, along with a reporter compatible with insect cells. In some embodiments, the reporter may be induced by insect cell viruses. In some embodiments, bacterial cells may be used, along with a reporter compatible with bacterial cells. In some embodiments, the reporter may be induced by bacteriophage infection. In some embodiments, plant cells may be used, along with a reporter compatible with plant cells. In some embodiments, the reporter may be induced by plant cell viruses. In some embodiments, mammalian cells may be used with a reporter compatible with mammalian cells, e.g., expression of fluorescent markers, enzymes, tagged proteins, or nucleic acids. In some embodiments, the mammalian cells are human cells. In some embodiments, the reporter may be induced by mammalian cell viruses.

In some embodiments, the assay readout may be a fluorophore expression. In some embodiments, the assay readout may be based on a Next Generation Sequencing (“NGS”) NGS-based signal or integrated NGS barcode. In some embodiments, the assay readout may be cell growth or cell death.

In some embodiments, a selectable marker may be used to select for cells transformed with nucleic acids encoding antibody and/or viral entry receptors.

As used herein, “selectable marker” refers to any molecule which permits the selection of a cell expressing the desired nucleic acid comprising nucleic acids encoding the selectable marker and a nucleic acid of interest. For example, in one embodiment, a cell of the instant disclosure expresses a nucleic acid comprising a nucleic acid encoding an antibody and encoding a fluorescent molecule, e.g., a fluorescent protein, (the selectable marker). Therefore, in the previous example, cells that are expressing the desired nucleic acid may be separated from cells not expressing the nucleic acid by use of methods known in the art to separate cells expressing a fluorescent molecule, e.g., fluorescence activated cell sorting (FACS). Other methods of separating cells expressing a selectable marker are known in the art including, but not limited to, antibody and magnetic bead separation. In some embodiments, the selectable marker confers a survival advantage to the cells expressing the nucleic acid of interest. For example, in some embodiments, the selectable marker confers resistance to antibiotics, e.g., blasticidin, Hygromycin B, puromycin, zeocin, G418/Geneticin, or others ⁽¹⁾. Thus, treatment of cells with the antibiotic for which molecules conferring resistance are encoded on the nucleic acid of interest, selects cells expressing the nucleic acid of interest and, therefore, acts as a selectable marker.

Thus, in some embodiments, a reporter comprises a selectable marker. However, though a reporter may, in some embodiments, comprise a selectable marker, a reporter functions to indicate to one of skill in the art practicing the disclosed methods, using the disclosed compositions or kits, that there is a change in the status of the cell in which the reporter is expressed, e.g., infection with a virus, presence of a cellular signaling event, lack of a cellular signaling event, etc.

In some embodiments, a selectable marker expressed by the cells may be used that enables selection for optimal protein or peptide function from a library of protein or peptide variants. In some embodiments, the selectable marker of secreted protein function may be a fluorescent protein not normally expressed in the cell line, including but not limited to green fluorescent protein (GFP), red fluorescent protein (RFP), yellow fluorescent protein (RFP), mCherry, blue fluorescent protein (BFP), cyan fluorescent protein (CFP) and others. In some embodiments, the selectable marker may induce expression of a surface protein for affinity-based selection, some examples might include CD19, CD4, CD34, and other surface proteins. In other embodiments, the selectable marker may include an enzyme that enables cell survival, including but not limited to apoptosis pathway genes, glutathione S-transferase, antibiotic resistance markers, Bleomycin, Adenosine deaminase, Xanthine-guanine phosphoribosyltransferase, or ⁽¹⁾ others. In some embodiments, the selectable marker may be read as a result of Cre-lox or CRISPR gene activation resulting in chromosomal changes. In some embodiments, an integrase may be used to insert genes into the cells for cloning libraries. In other embodiments, stable cell pools may be used to generate libraries from transfected plasmids. In other embodiments, secreted protein libraries may be generated using an integrase. In other embodiments, secreted protein libraries may be generated using a transposase. In some embodiments, the readout of the assay may be based on sequencing of cell populations after screening. In some embodiments, that assay readout may involve the identification of DNA barcodes encoded by the antibodies and/or the virus or viral infection model as a unique identifier of the antibody or viral infection variant, respectively. In other embodiments, the readout of the assay may be based on fluorescent markers and sorting via flow cytometry.

In some embodiments, the secreted polypeptide may be an antibody variant. In some embodiments, the antibody may be of one or more of the following formats: IgG, IgM, IgA, Fab, ScFv, Fab2′. In some embodiments, the antibody may be a bispecific antibody. In other embodiments, the antibody may be a trispecific antibody. In some embodiments, the secreted proteins may comprise antibody native heavy:light ^{(2,3,4)} pairs.

In some embodiments, the secreted polypeptides may comprise randomly paired heavy and light chains. In some embodiments, the antibody heavy:light expression may be on the same mRNA transcript. In other embodiments, the antibody heavy and light chains may be expressed on separate mRNAs. In some embodiments, a bidirectional promoter may be used in between the heavy and light⁽³⁾ chain mRNAs.

In some embodiments, the secreted polypeptides comprise antibodies found in antibody gene libraries derived from human patients developed by screening of native human immune libraries. In other embodiments, the antibody gene libraries may be derived from animal sources, including mouse, transgenic mouse, camellid, shark, non-human primate, guinea pig, or other animals. In some embodiments, the antibody gene libraries may be synthetically generated. In certain embodiments, the libraries may comprise synthetically generated libraries with introduced diversity (for example, via targeted mutagenesis, site-saturation mutagenesis, DNA shuffling, error-prone PCR, somatic hypermutation, or other diversity introducing mechanisms). In some embodiments, the protein library may be based on antibody genes with known activity. In some embodiments, the disclosed screening methods may be used to select for improved potency, selectivity, or breadth of diversified libraries derived from antibodies with known baseline activity. In some embodiments, a secreted polypeptide may be selected for its ability to agonize or antagonize cellular receptors expressed by any species, including but not limited to mouse, non-human primate, guinea pig, ferret, pig, and human.

In some embodiments, the secreted test polypeptide comprises antibodies derived from subjects suffering from, or being tested for, autoimmune disease. As one example, a cell (e.g., in a droplet) may express a membrane-bound target known to be an autoimmune antigen associated with a specific autoimmune disease. In other embodiments, the membrane-bound target may comprise a library of possible autoimmune polypeptides. In other embodiments, an immune receptor may be expressed as a membrane-bound target, and an antigen may be secreted as a test polypeptide. The cell may be isolated inside a compartment. Upon detection of binding events between the secreted test polypeptide (e.g., autoantibody) to the membrane-bound target, the cell is isolated and the gene encoding the membrane-bound target and the secreted polypeptide are sequenced and identified. In this manner, the natural ligand to a large set of autoimmune antibodies can be identified against a library of autoimmune antigens, greatly aiding in the diagnosis and typing of autoimmune disease in the subject. Such antibodies may also be useful for therapeutic purposes and for autoimmune drug discovery.

In some embodiments, the secreted test polypeptide and the membrane-bound target can be applied to map autoimmune responses for T cell receptor (TCR):peptide major histocompatibility complex (pMHC) binding pairs. The mechanics for a TCR-pMHC interaction assay include a secreted test polypeptide and a membrane-bound target, and is implemented the same as for an antibody-antigen interaction, or for any other interaction between a membrane-bound target and a secreted polypeptide. In some embodiments, a cell may each express a pMHC gene as a membrane-bound target, while secreting soluble TCR polypeptide with potential to bind to the expressed pMHC genes. In other embodiments, the cell may express a TCR polypeptide as a membrane-bound target, while with pMHC as a secreted polypeptide. The cell may be isolated inside a compartment. Upon detection of binding associations between the secreted test polypeptide (in some embodiments, a soluble TCR) to the membrane-bound target (in some embodiments, a pMHC), the cell is isolated and the gene encoding the membrane-bound target and the secreted polypeptide are sequenced and identified. In this manner, the natural ligand to a large set of T cell receptors can be identified against a library of pMHC antigens, greatly aiding in the diagnosis and typing of T cell function a subject or a T cell library. Such antibodies may also be useful for therapeutic purposes and for drug discovery in settings such as autoimmunity, cancer, viral infections, and other diseases.

In some embodiments, the membrane-bound target is associated with the cell via a bridging molecule, chemical, particle, or other molecular moiety to link a secreted polypeptide to the cell membrane. Approaches to combine a cell with a membrane-bound target are known to those skilled in the art.

In some embodiments, the secreted test polypeptide, or secreted test polypeptide library variants may have some baseline activity, and the functional screen described is used to improve its potency, selectivity, or breadth of activity. In some embodiments, the starting protein or peptide library may have uncharacterized activity, and the functional assays described herein are used to characterize the functional activities of variants in the protein or peptide library and select for desired functional variants.

In some embodiments, the cell may be engineered to introduce genetic diversity to the secreted polypeptides between selection rounds. Several mechanisms for introducing genetic diversity are known to individuals skilled in the art, including the expression of activation-induced cytidine deaminase (AID), expression of an error-prone polymerase, or the use of an orthogonal plasmid replication system. In some embodiments, a library of variants can be generated prior to the detection step. In some embodiments, a library of variants can be generated concurrently with the detection step. In some embodiments, mechanisms to introduce gene diversity are implemented concurrently with the detection step.

In some embodiments, the secreted test polypeptide expression promoters may be varied to modulate the secreted protein concentrations, where stronger promoters influence the secreted concentration. Weaker promoters may be used to enable more potent secreted protein selection. In some embodiments, the amount of time of protein secretion may be varied to similarly adjust secreted protein concentrations. In some embodiments, by way of example, a shorter incubation time prior to the addition of virus can provide a lower soluble polypeptide concentration in supernatant, thereby selecting for more potently active or protective secreted molecules.

In some embodiments, the functional assay resulting in a reporter (e.g., GFP expression) may derive from a virus infection, and the assay comprises a virus neutralization assay, wherein the secreted polypeptide expressed and secreted by the cell includes an antibody, or antigen binding fragment.

In some embodiments, the functional assay may comprise the binding and activation or signal transduction via a cellular receptor (for example, a G-protein coupled receptor, a T cell receptor, a chimeric antigen receptor, an apoptosis marker, an immunomodulator such as PD-1, LAG-3, TIM, 4-1BB, or others). In such embodiments, the functional assay may comprise a screen for secreted proteins that can activate the cellular receptor and induce signal transduction. The signal transduction event could be linked to any reporter (e.g., fluorescent protein expression, apoptosis markers, cell surface marker expression, Cre-Lox or CRISPR expression, or mRNA-based markers) that would enable readout of the secreted protein's functional effect on the desired cellular receptor activation. In some embodiments, the secreted protein may block the surface receptor and prevent its activation in the presence of activating moieties (e.g., a ligand naturally produced by the cell, engineered to be produced by the cell, or added to contact the cell), resulting in a functional readout, e.g., a reporter. In other embodiments, the secreted protein may directly activate the surface receptor.

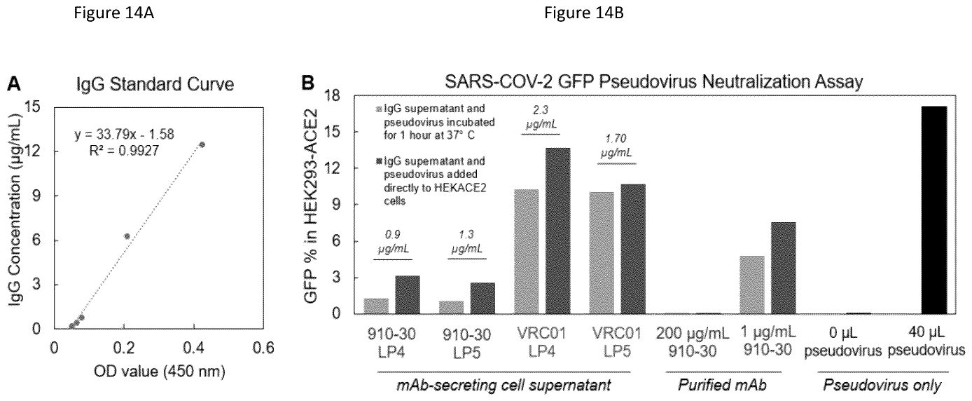

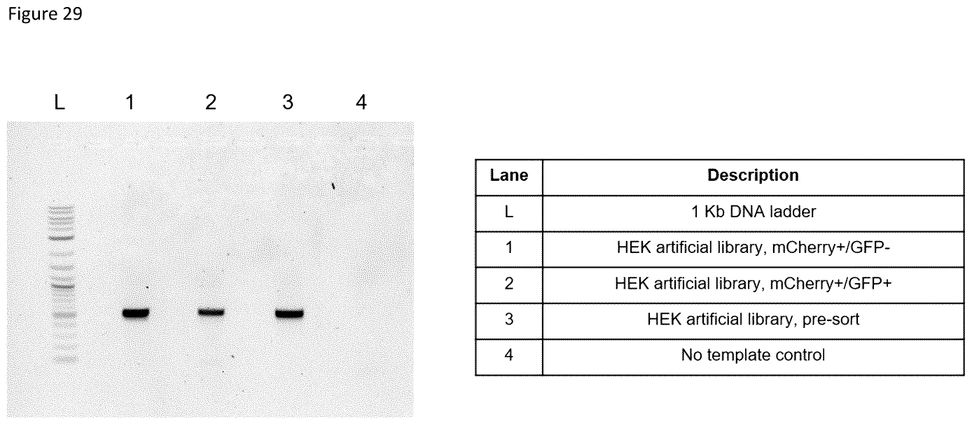

In some embodiments, single cells are isolated into compartments for functional screening of the secreted proteins. In some embodiments, the compartments may be 96- or 384 well plates. In some embodiments, the compartments may be printed ^{(4,5)} microwells, open microchambers, or Nanopens™. In other embodiments, the compartments may be emulsion ⁽⁶⁾ droplets (See, for example, FIGS. 15, 16, 18, 28, and 29). In some embodiments, additional reagents may be added to the compartments after a certain amount of time has passed for the desired secreted test polypeptide to accumulate inside droplets. In well plates, reagent addition may occur by fluid addition. In printed ^{(4,5)} microwells, open microchambers, or Nanopen™ reagent addition may be accomplished by washing or fluid flow near the unsealed compartment. In emulsion droplets, reagent addition may occur by droplet merger. In some embodiments, droplet merger may be accomplished by electrocoalescence (See Example 26, FIG. 29), printed pillar resistance, or other means of induced droplet fusion. In certain embodiments, the addition of reagents after the initial encapsulation of a library cell comprising a secreted protein variant may be unnecessary.

In some embodiments, the reagents added to the compartments may contain a virus or pseudovirus, in which case the assay may be a virus neutralization assay. In some embodiments, only a single virus or pseudovirus may be added. In other embodiments, multiple viruses or viral variants may be added. In some embodiments, the viruses or pseudoviruses may be barcoded with different selection markers to identify the infecting virus. In some embodiments, the viruses or pseudoviruses may be barcoded, tagged, or labeled with one or more different fluorescent markers, DNA barcodes, or membrane-bound targets. In some embodiments, the virus or pseudovirus infection may cause cell death, and only cells encoding protective secreted proteins that neutralize the virus or pseudovirus can survive after the assay.

A longstanding challenge in antibody engineering and discovery is the need to identify agonistic or antagonistic antibodies against membrane proteins. Manipulating cellular behavior using membrane protein interactions is a goal in modern medicine, including in cancer biology and in autoimmune disease treatments. Some examples of membrane protein targets include the surface markers 4-1BB, OX40, PD-L1, PD-1, CTLA-4, LAG-3, G protein-coupled receptors (GPCR), and ion channels. Two of the biggest challenges to the discovery of antibodies targeting membrane proteins or targets include: 1) the ability to express and purify soluble versions of the membrane-bound protein, because membrane proteins are non-native when expressed in a soluble format, and 2) it is technically complex to screen for the function of antibodies that bind to native, membrane-bound versions of the proteins⁽⁷⁾, rather than simply screening for binding. The presently described approach for connecting together a secreted test protein expression in the same cell as membrane surface expression of target proteins elegantly addresses these two traditional challenges because there is no need to express and purify the membrane protein or membrane-bound target in a non-native solubilized format for screening, and also because the use of cell-based activation markers (such as fluorescence marker expression or luciferase expression) can provide a direct readout of the functional activity of the test protein that is secreted by a single cell. Thus, the approach described here for secreted protein analysis can be used for the membrane targets, including surface proteins and receptors like 4-1BB, OX40, PD-L1, PD-1, CTLA-4, LAG-3, G protein coupled receptors (GPCR), and ion channels.

In some embodiments, the secreted protein activity may be an agonist or antagonist of receptor activity. In certain embodiments, a reagent, could be added to the compartments and may be e.g., a receptor agonist, for example PD-L1 for the PD-1 receptor. In other embodiments, the reagents added to the compartments may be receptor antagonists that prevent receptor activation upon binding. In some of these embodiments, receptor activation is linked to reporter expression, for example, fluorescent moiety expression to screen cells for their ability to secrete an antibody regulating protein receptor activity. Certain cell lines with receptor activation reporters comprising fluorescent signals, luciferase signals, or other signals to indicate receptor activity have been generated and may be used for this purpose once they are suitably transformed with libraries of secreted proteins for analysis and selection. Example commercially available cell lines having receptor activation reporters include cell lines responding to 4-1BB, OX40, PD-1/PD-L1, CTLA-4, or LAG-3 ligand/agonist antibody stimulation that are available from the Promega Corporation.

### Compositions

In one aspect of the current disclosure, compositions are provided. In some embodiments, the compositions comprise: a single, isolated, genetically engineered cell, wherein the cell presents a membrane-bound target; and wherein the cell is engineered to: (i) secrete a secreted polypeptide; and (ii) express a reporter molecule if the test polypeptide activates the membrane-bound target.

In some embodiments, the compositions comprise a single, isolated, genetically engineered cell, wherein the cell presents a membrane-bound target; and wherein the cell is engineered to: (i) secrete a secreted polypeptide; and (ii) express a reporter molecule if the test polypeptide does not activate the membrane-bound target.

In some embodiments, the compositions comprise a single, isolated, genetically engineered cell, and optionally a test reagent, wherein the cell presents a membrane-bound target, and wherein the cell is engineered to: (i) secrete a secreted polypeptide; and (ii) express a reporter molecule if one of the test polypeptide or the test reagent activates the membrane-bound target.

In some embodiments, the compositions comprise a single, isolated, genetically engineered cell, and optionally, a test reagent comprising a reporter molecule, wherein the cell presents a membrane-bound target; wherein the test reagent is capable of binding the membrane-bound target presented by the cell, forming a reagent-receptor complex, and wherein the test reagent gains entry into the cell when the reagent-receptor complex is formed; wherein the cell is engineered to: (i) secrete a secreted polypeptide.

In some embodiments, the cell comprises a mammalian cell, an insect cell, an avian cell, a yeast cell, a plant cell, or a bacterial cell. In some embodiments, the cell comprises a human cell.

In some embodiments, the membrane-bound target comprises an endogenous receptor. In some embodiments, the cell is engineered to express the membrane-bound target. In some embodiments, the membrane-bound target comprises a heterologous protein.

In some embodiments, secretion of the test polypeptide is constitutive. In some embodiments, secretion of the test polypeptide is inducible.

In some embodiments, the single, isolated, genetically engineered cell is in a well of a multi-well plate. In some embodiments, the single, isolated, genetically engineered cell is in a chamber of a microchip. In some embodiments, the single, isolated, genetically engineered cell is in a microfluid droplet, such as an emulsion droplet. In some embodiments, the single, isolated, genetically engineered cell is in a Nanopen™

In some embodiments, the reporter molecule comprises a fluorescent marker, an enzyme, a tag protein, or a nucleic acid sequence. In some embodiments, the reporter molecule comprises a nucleic acid sequence, optionally a barcode sequence. In some embodiments, the reporter molecule comprises a fluorescent moiety.

In some embodiments, the secreted polypeptide comprises a variant of the receptor ligand. In some embodiments, the variant is derived from a library of ligand variants. In some embodiments, the secreted polypeptide comprises a potential receptor agonist or antagonist.

In some embodiments, the test reagent comprises an agonist or an antagonist of receptor activation. In some embodiments, the test reagent comprises the membrane-bound target ligand, and the secreted polypeptide is derived from a library of potential agonists or antagonists of receptor activation.

In some embodiments, the secreted polypeptide comprises an antibody or antigen binding fragment thereof. In some embodiments, the antibody or antigen binding fragment is derived from a library of antibodies, or antigen binding fragments.

In some embodiments, the test reagent comprises a virus, and the membrane-bound target comprises a component of viral entry into the cell. In some embodiments, the virus is on or more selected from Coronavirus A, B, C, or D, Flavivirus, Lentivirus, Influenza A, B, or C. In some embodiments, the virus selected from HIV, SARS-CoV-2, and Yellow Fever Virus. In some embodiments, the virus comprises a SARS-CoV-2 virus, and wherein the membrane-bound target comprises a human angiotensin-converting enzyme 2 (hACE2). In some embodiments, the cell is engineered to express Transmembrane Serine Protease 2 (TMPRSS2).

In some embodiments, the cell is also engineered to introduce new gene diversity to the secreted polypeptide between selection rounds. Several mechanisms for introducing genetic diversity are known to individuals skilled in the art, including the expression of activation-induced cytidine deaminase (AID), expression of an error-prone polymerase, or the use of an orthogonal plasmid replication system. In some embodiments, a library of variants can be generated prior to the detection step. In some embodiments, a library of variants can be generated concurrently with the detection step. In some embodiments, mechanisms to introduce gene diversity are implemented concurrently with the detection step.

### Kits

In another aspect of the current disclosure, kits are provided. In some embodiments, the kits comprise: (a) a vector for the expression of a secreted polypeptide into a cell; (b) a vector encoding a reporter molecule, expression of which is activated if the secreted polypeptide activates a membrane-bound target presented on the cell, optionally, wherein one or more of the vectors are expression vectors, or, optionally, wherein one or more of the vectors are integration vectors.

In some embodiments, the kits comprise: (a) a vector for the expression of a secreted polypeptide in a cell (b) a vector encoding a reporter molecule, expression of which is activated if the secreted polypeptide does not activate a membrane-bound target, optionally, wherein one or more of the vectors are expression vectors, or, optionally, wherein one or more of the vectors are integration vectors.

In some embodiments, the kits comprise: (1) a test reagent and (2) (a) a vector for the expression of a secreted polypeptide into a cell (b) a vector encoding a reporter molecule, expression of which is activated if either the secreted polypeptide or test reagent activates a membrane-bound target, optionally, wherein one or more of the vectors are expression vectors, or, optionally, wherein one or more of the vectors are integration vectors.

In some embodiments, the kits comprise: (1) a test reagent comprising a reporter molecule and (2) (a) a vector for expressing a test polypeptide in a cell, optionally, wherein the vector is an expression vector, or, optionally, wherein the vector is an integration vector.

In some embodiments, the one or kits comprise one or more vectors for the expression of (c) a membrane-bound target. In some embodiments, the secreted polypeptide is operably linked to a promoter. In some embodiments, the promoter is a constitutive promoter. In some embodiments, the promoter is an inducible promoter. In some embodiments, the reporter molecule comprises one or more of a fluorescent marker and a barcode. In some embodiments, the reporter molecule is operably linked to an inducible promoter. In some embodiments, the test reagent comprises a virus or a pseudovirus. In some embodiments, the virus is selected from one or more of a Coronavirus A, B, C, or D, Flavivirus, Lentivirus, and Influenza A, B, or C. In some embodiments, the virus is selected from HIV, SARS-CoV-2, and Yellow Fever Virus. In some embodiments, the pseudovirus comprises a peptide, polypeptide, or protein derived from one or more of a Coronavirus A, B, C, or D, Flavivirus, Lentivirus, or Influenza A, B, or C. In some embodiments, the pseudovirus comprises a peptide, polypeptide, or protein derived from HIV, SARS-CoV-2, or Yellow Fever Virus. In some embodiments, the secreted polypeptide comprises an antibody, or a portion thereof. In some embodiments, the secreted polypeptide includes a single chain variable fragment (scFv) or a nanobody.

In some embodiments, the kits comprise: (1) a vector for expressing a secreted polypeptide, (2) a genetically engineered cell comprising: (a) a nucleic acid encoding a reporter, expression of which is activated if the secreted polypeptide activates a membrane-bound target.

In some embodiments, the kits comprise: (1) a vector for expressing a secreted polypeptide, (2) a genetically engineered cell comprising: (a) a nucleic acid encoding a reporter, expression of which is activated if the secreted polypeptide does not activate a membrane-bound target.

In some embodiments, the kits comprise: (1) a vector for expressing a secreted polypeptide, (2) a genetically engineered cell comprising: (a) a nucleic acid encoding a reporter, expression of which is activated if the secreted polypeptide activates a membrane-bound target, and optionally, (3) a test reagent.

In some embodiments, the kits comprise: (1) a vector for expressing a secreted polypeptide, (2) a genetically engineered cell (3) a test reagent comprising a reporter molecule, wherein the test reagent is capable of binding a membrane-bound target presented by the cell, forming a reagent-receptor complex, and wherein the test reagent gains entry into the cell when the reagent-receptor complex is formed.

In some embodiments, the genetically engineered cell further comprises: (c) a nucleic acid encoding a heterologous membrane-bound target.

As used herein, “expression vector” refers to a vector that is used to express a nucleic acid sequence of interest encoded on the vector. In some embodiments, the expression vector expresses the nucleic acid as an RNA product. In some embodiments, the RNA expression product is translated to a polypeptide or protein.

As used herein, “integration vector” refers to a vector that is used to integrate a nucleotide sequence of interest into the genome of a target cell. Exemplary methods of integrating a nucleic acid into the genome of a cell are known in the art, e.g., CRISPR Cas9-based homologous recombination, retroviral or lentiviral transduction.

As used herein, a “library of genes” refers to a collection of cells comprising or encoding four or more gene variants. Gene variants range in differences from single-codon mutations to entirely different genes from different protein families. Libraries of secreted proteins could include ligands, extracellular protein domains, antibodies, and other proteins.

As used herein, a “library of surface targets” refers to a library comprising or encoding that may include membrane proteins, fusion proteins, antibodies, other proteins, chemical moieties, polymers, oligomers, DNA, RNA, glycans, or other molecular and chemical agents.

As used herein, a “cell” is defined as a lipid containing membrane that is capable of encapsulating a volume of aqueous medium. A cell may include a living cell (e.g., eukaryotic and prokaryotic), an exosome, or an artificial cell.

As used herein, a “membrane” is defined as a lipid-containing layer that can incorporate membrane-associated proteins (e.g., membrane-bound targets). The membrane may include cellular membranes from living cells and artificial cells.

As used herein, “endogenously express” is defined as to be naturally expressed. For example, a cell line that endogenously expresses a protein does so without artificial intervention.

### Large-Scale Screening of Polypeptide-Target Interactions

Antibodies are the principal adaptive immune protection mechanism against extracellular antigens, and every individual generates millions of distinct antibody molecules, each comprised of a unique heavy and light chain gene. The repertoire of antibodies contains tremendous diversity and is almost entirely unique to each human, with only a small fraction of shared sequences even across identical twins⁽²⁷⁾. With >10⁸ unique antibodies per individual and a continually evolving antibody repertoire, immunologists confront major technical challenges to understand the scope and quality of individual antibody immunity. Sequencing technologies have advanced significantly in the past 15 years; however, we remain unable to identify many desirable functional features for antibodies either from native immune responses in humans or laboratory models, and also in synthetic antibody libraries. While sequence technology has progressed rapidly, mapping antibody responses to their diverse antigen targets remains low-throughput and time consuming and is inadequate in numerous ways for large-scale understanding and precision engineering of antibody function⁽²⁸⁻³¹⁾.

Specifically, drug discovery against membrane proteins is a major unmet need. Traditional antibody discovery technologies have major difficulties isolating antibodies against proteins in a membrane-bound format because the membrane antigens are difficult to isolate, purify, and stain at scale. Many membrane proteins are unable to be expressed in soluble forms, and this leads to a difficulty in discovering antibodies effectively against these membrane targets⁽³²⁻³⁵⁾.

Additionally, antibodies can be identified based not only on their binding properties, but also on their ability to engage fragment crystallizable (Fc) effector functions. Fc effector functions constitute a range of different mechanisms by which the antibody proteins engage with other cells and proteins in the immune system in vivo. Fc effector proteins include (but are not limited to) the Fc gamma receptors (e.g., FcgRI, FcgRIIa, FcgRIIb, FcgRIIIa, FcgRIIIb)⁽³⁵⁻³⁶⁾. Complement engagement is also a known function of antibodies, and the ability of antibodies to bind and engage the C1 complex (especially via antibody interactions with C1q) is has been shown for many applications⁽³⁷⁾. Target indications for understanding, mapping, and engineering antibody effector functions and complement engagement include antibodies for the treatment and prevention of infectious diseases, cancers, and autoimmunity.

Numerous antibody discovery technologies have been reported. Cloning pipelines have been invented for antibody expression in yeast display that enabled precision fluorescence-activated cell sorting (FACS) and next-generation sequencing (NGS) analysis to map antibody functional recognition against defined antigens². These technologies use display libraries and screening against solubilized antigens presented in various formats, including: (1) soluble, purified antigens, (2) virus-like particles displaying membrane proteins (3) lipid nanodiscs displaying membrane proteins, and (4) detergent-solubilized antigens⁽³⁸⁻⁴²⁾.

The above listed solutions are compatible both with screening for antibodies in display systems by FACS, and with magnetic-activated cell sorting (MACS) but have disadvantages including the time and expense required to purify, antigens. They are also compatible with the staining of B cells expressing B cell receptors using oligonucleotide-coded soluble antigens for transcriptomics-based deconvolution of antibody:antigen pairs⁽⁴³⁻⁴⁴⁾. Any of the above approaches can be combined with MACS or FACS for reasonable detection of antibodies targeting membrane proteins that can be solubilized and presented in the above or similar solubilized formats.

However, many membrane proteins are unable to be expressed in any soluble form, often due to structural features (for example, short regions between membrane spanning domains). Several alternative approaches also exist for antibody discovery against membrane proteins, including methods for cell-based screening of display libraries (expressed in phage, yeast, and bacteria most commonly) that involve screening these display libraries for binding to antigens expressed by other cells (sometimes called biopanning)⁽⁴⁵⁻⁵²⁾. DNA-barcoded peptide screening and bead-based approaches have been reported but are limited by the low protein complexity that can be achieved via in vitro transcription and translation⁽⁵³⁾ and would be unlikely to be effective for most complex proteins like antibodies.

However, these approaches have major limitations for many antibody discovery and screening needs. Cell-based biopanning is cumbersome to perform and is not successful against every antigen attempted. Oligonucleotide-labeled solutions have a low throughput (only a few thousand B cells analyzed in a typical sample), require solubilized antigens, and often require full transcriptomic analysis. Technologies have also been developed for screening of antibodies secreted by antibody-secreting cells inside droplets⁽⁵⁴⁻⁵⁶⁾. However, these technologies require complex microfluidics-based sorting because the signal to be detected is located on other beads or cells that are co-localized inside the same droplet, and the signaling mechanism is not a property of the cell itself. In other examples, multiple cells are encapsulated in the droplet, and a second cell is used as a detection agent⁽⁵⁷⁻⁵⁸⁾. Thus, any cell sorting necessary must be performed before the emulsion droplets are broken because they contain both the cell and a signaling agent.

In certain cases, it is desired to obtain a polypeptide that will bind to a membrane protein, but not necessary activate or inactivate that protein's signaling or other function. For example, it may be desired to target an inert site or to label the protein for a diagnostic or theranostic application, rather than affect membrane protein behavior directly. To address this challenge, a method is desired that will somehow detect the polypeptide binding event without analysis of downstream protein signaling or activation capacities. We describe here a new system here that can detect the binding of polypeptides, such as antibodies or other binding polypeptides, on a cell membrane-bound protein or target, without any activation activities, and where the signal remains detectable by the cell itself. This work enables large-scale and high-throughput polypeptide discovery against protein targets, for a range of purposes desirable to scientists but that do not necessarily rely on signaling.

The above technologies may have difficulty screening antibody clones for different Fc effector functions, such as Fc gamma receptor engagement or complement system engagement. These Fe effector functions are needed for antibody or antibody domain fusion protein efficacy in a number of disease applications, including for polypeptides targeting cancer or tumor-associated antigens, and for polypeptides against infectious disease targets. In some cases, it may be desirable to identify antibodies or antibody domain fusions with weak Fc effector functional engagement, for example to block (inactivate) a receptor or to label a protein for diagnostic or theranostic applications, without engaging immune responses.

In addition to the issues with the aforementioned and parallel advances, large-scale protein:target mapping remains difficult, including for antibody:antigen interactions because: 1) polypeptides used for FACS must be individually expressed, purified, biotinylated, labeled with specific barcodes, undergo QC before use with precious/labile human B cells, and have a limited shelf life³; 2) many polypeptides require complex mammalian expression and glycosylation, especially for viruses and autoimmune targets; and 3) functional assays require soluble polypeptide (e.g. virus neutralization, Fc effector engagement, etc.) and are incompatible with native B cells or yeast display. Costly single-cell transcriptome technologies (e.g. 10× Genomics/LIBRA-seq³) also limit researchers to just a few thousand B cells per sample, beyond which the required instruments & kits are cost-prohibitive for all but the wealthiest labs. Furthermore, LIBRA-seq requires surface-expressed antibodies and cannot analyze secreted polypeptides, for example the antibodies expressed by plasma cells, despite the role of plasma cells for serum antibody production. DNA sequencing capacity also prohibits mapping of 100,000 antibody:antigen pairs via transcriptomics (even if 10,000 targeted antigens could somehow be reliably expressed in solution, purified, individually barcoded, and stored.) Technical workarounds are low-throughput, including cumbersome single-cell culture in microfluidic chips⁽⁵⁹⁻⁶¹⁾, or antibody expression/purification followed by live cell assays in 96-well plates. Yeast mating for protein:target screening has been reported, but it lacks mammalian glycosylation, cannot be used for soluble virus neutralization or Fc engagement assays, lacks analysis of natively paired antibodies, and is proprietary & inaccessible for many researchers⁽⁶²⁾. Surface display technologies and B cell sorting similarly cannot analyze antibody properties like Fc effector engagement and virus neutralization⁽⁶³⁾. The above limitations have stymied scientific progress because our data are limited for the analysis of protein:target and antibody:antigen recognition at very high throughput. A technology is required both for the detection of polypeptide binding alone, and also for the detection of Fc engagement and complement engagement, which is compatible for membrane-expressed polypeptides that will map polypeptide:target and antibody and antigen binding at large scale, and to analyze antibody fusion domain effector functions.

### Binding to Membrane-Bound Targets

In some embodiments, the binding between a secreted polypeptide and a membrane-bound target is detected. The secreted polypeptide may be secreted from a genetically modified cell, and the membrane-bound target may be incorporated upon, and/or expressed by, the same genetically modified cell. The cell, the secreted polypeptide, and the membrane-bound target may be included within a single compartment. In some embodiments, the compartment may include a droplet, a well (e.g., of a plate, such as a 96-well plate), a spatially separated cell culture condition, or an encapsulation, such as a gel encapsulation.

In some embodiments, at least one of the secreted polypeptides or the membrane-bound target includes an antibody, or an antibody fragment. In some embodiments, the cell stably expresses a single membrane-bound target and encodes a library of different secreted proteins. The cell may also express a single secreted protein and encode a library of different surface proteins. The cell may be engineered to endogenously or exogenously express either the secreted polypeptide(s) or the membrane-bound target (s). For example, in a screening method for detecting a binding of an antibody Fc effector protein to a secreted polypeptide bound to a membrane-bound target on the surface of a genetically engineered cell, the cell may endogenously express (e.g., naturally express) the target (e.g., a polypeptide), that binds to the cell membrane at the cell surface. In another example, in a screening method for detecting a binding of a complement protein to a secreted polypeptide bound to a membrane-bound target on the surface of a genetically engineered cell, the cell may endogenously express the target that binds to the cell membrane at the cell surface.

In embodiments, the cells may be sorted based on the binding of the secreted polypeptide to the membrane-bound target. For example, the cells may be sorted via MACS or FACS. In embodiments, the secreted polypeptide and/or membrane-bound target may be identified by DNA sequencing of the sorted cells (e.g., via sequencing the gene and/or barcode for the secreted polypeptide and/or target-bound polypeptide). Cell sorting may be performed via identification of a secreted polypeptide detection agent (e.g., a labeled antibody, labeled protein, or other detectable label).

In embodiments, the cells may be sorted based on a detection of an internalization signal. It is understood that cell membrane receptors and other cell surface proteins (e.g., membrane-bound targets) often internalize or otherwise become sequestered upon binding to ligands (e.g., drugs). For detection of binding based on internalization, the secreted polypeptide or membrane-bound target may include a label that changes one or more characteristics, such as fluorescence, depending on whether the secreted polypeptide or membrane-bound target has been internalized. Labels used for detection of internalization include but are not limited to fluorogen activating proteins, cell dyes with limited permeability, pH-sensitive dyes and commercially available tags such as SNAP-tag® and CLIP-tag™ available from New England Biolabs. For example, a cell may express a membrane-bound target polypeptide that is tagged with a fluorescent peptide having low extracellular fluorescence, but upon ligand binding and subsequent internalization, the fluorescent tag becomes highly fluorescent. This fluorescence may be activated by low pH inside endosomes after endocytosis, as one possible mechanism for fluorescence activation. The difference in fluorescence can then be quantified by FACS or other methods. Additional methods for detection of internalization can be used as would be known to those skilled in the art.

In embodiments, the cells may be sorted based on a detection of a degradation signal (e.g., a signal that is degraded, diminished, or absent). For example, the signal may be based on a proteolysis targeting chimera (PROTAC) signal. PROTACs are heterobifunctional molecules composed of two active domains and a linker, capable of removing specific unwanted proteins by inducing selective proteolysis. Typical PROTACs consist of two covalently linked protein-binding molecules: one capable of engaging an E3 ubiquitin ligase, and another that binds to a target protein meant for degradation. Recruitment of the E3 ligase to the target protein results in ubiquitination and subsequent degradation of the target protein. The selective degradation catalyzed by PROTACs can also be used to selectively degrade or diminish the selective polypeptide and/or membrane-bound target once the secreted polypeptide and membrane-bound target have bound. For example, the membrane-bound target may be a fusion protein that includes a fluorescent polypeptide (GFP), and the secreted polypeptide may be a PROTAC that binds to the fusion protein. Once the secreted polypeptide binds the membrane-bound target, the PROTAC may then bind the fusion protein, as well as E3 ligase to cause ubiquitination. The fusion protein is signaled for degradation and the GFP signal is degraded is diminished, leading to a reduction of signal that can be detected via flow cytometry or via other means. PROTACs may include any type of dual binding compounds or polypeptides including but not limited to multispecific antibodies, T cell receptors, VHH, scFv, and nanobodies. In some embodiments, the loss of the membrane-bound target is detected by a specific antibody staining for the surface target, or using the membrane-bound target's native ligand. In some embodiments, flow cytometry or magnetic cell sorting can be used to separate cells that display a membrane-bound target from other cells in the population that secrete a PROTAC which efficiently degrades or diminishes the membrane-bound target. In some embodiments, the membrane-bound target and the secreted polypeptide are sequenced to reveal the paired sequence of both the PROTAC and its target. In some embodiments, a library of PROTACs may be screened against a library of membrane-bound targets for rapid identification of both PROTACs and target antigens together.

In embodiments, the cell is compartmentalized (e.g., into single isolated genetically engineered cells). The compartmentalization may be achieved via a microwell plate, nanopen, nanowell, droplet, encapsulation in a gel, or other means of isolating single cells from one another.

In embodiments, the method may identify binding of secreted polypeptides to surface moieties that are not proteins, such as DNA, glycans, and chemical antigens. For example, moieties may be conjugated to the cell surface in well plates, then encapsulated into droplets to analyze binding of secreted polypeptides to the moieties.

In embodiments, the secreted polypeptide includes an antibody, VHH, nanobody, or any domains from an antibody molecule. In embodiments, the method may include identification of antibody Fc effector functions (e.g., antibody Fc effector protein binding) when an antibody, or a fusion protein including antibody protein domains, is secreted by a single cell in a compartment, and the antibody Fc effector engagement is detected from the polypeptides bound to the cell surface. Both antibody Fc effector protein engagement and the binding of the secreted polypeptide to the membrane-bound target may be performed in parallel. Detection of antibody Fc effector protein engagement and subsequent sorting may be performed via identification of a detection agent associated with the antibody Fc effector protein bound to the secreted polypeptide that is itself bound to the membrane-bound target (e.g., the detection agent including a labeled antibody, labeled protein, or other detectable label). In embodiments, the secreted polypeptide and/or the membrane-bound target includes a T cell receptor (TCR).

In embodiments, the secreted polypeptide includes a peptide:MHC complex. For example, the secreted polypeptide may include an MHC complex that has been exposed to a peptide (in vivo or ex vivo), where the MHC complex subsequently binds the peptide. In another example the MHC complex and peptide may be formed as a single polypeptide, with the MHC and peptide expressed from a single construct encoding both the MHC and peptide linked by a protein linker sequence. Once translated, the peptide, linked to the MHC, subsequently folds and intramolecularly binds to the MHC. In some embodiments, the membrane-bound target includes a peptide:MHC complex.

In embodiments, the antibody Fc effector protein detection agent includes one or more of monomeric FcgRx, FcaRx, FcmRx, FceRx proteins, other Fc domain binding polypeptides, another cell, or a synthetic fusion or dimeric form of Fc receptor engagement polypeptides, any one of which domains could include an FcgRx, FcaRx, FcmRx, FceRx protein. For example, the antibody Fc effector protein detection agent may include monomeric FcγRI, FcγRIIa, FcγRIIb, FcγRIIc, FcγRIIIa, FcγRIIIb, FcαRI, FcμR, FcaR, FcμR, FcεRI, FcεRII/CD23, DC-SIGN, Fcα/μR, FcRn, or other Fc effector proteins, or a partial polypeptide sequence thereof. In another example, the antibody Fc effector protein may include at least one polypeptide sequence derived from at least of one of: FcγRI, FcγRIIa, FcγRIIb, FcγRIIc, FcγRIIIa, FcγRIIIb, FcαRI, FcμR, FcαR, FcμR, FcεRI, FcεRII/CD23, DC-SIGN, Fcα/μR, FcRn, or other Fc effector binding protein genes.

In some embodiments of the method, complement engagement is detected in parallel to Fc receptor engagement and/or detection of the secreted polypeptide to the membrane-bound target. The antibody Fc effector protein detection agent may also include a virus, such as a phage or a lentivirus.

In some embodiments, the method includes identifying of antibody complement engagement functions (e.g., complement protein binding) when an antibody, or polypeptide containing a polypeptide sequence derived from an antibody gene fragment, is secreted by a single cell in a compartment, and complement activity is detected inside the compartment. Detection of polypeptide complement engagement functions, or complement protein binding, and subsequent cell sorting may be performed via identification of a complement protein detection agent bound to a complement protein that is itself bound to the secreted polypeptide, (e.g., a labeled antibody, labeled protein, or other detectable label). Complement proteins may include any proteins and/or other entities of the complements system or cascade including but not limited to C1q, C1s, C1r, a C1 complex, C1 complex proteins, C2b, C4a, C4b, C3, C3a, C3b, C5, C5a, C5b, C6, C7, C8, C9, Bb, Ba, Factor D, Factor B, Factor I, Factor H, and associated binding partners, factors, enzymes, and proteases.

It should be understood that the detection, sorting, and identification of the secreted polypeptide and membrane-bound target in regard to polypeptide-target binding, Fc effector protein engagement, and complement engagement function may be performed via the detection (e.g., fluorescent probes), sorting (e.g., MACS or FACS), and identification (e.g., sequencing) methods as described herein.

In embodiments, the detection step may occur either before or after recovering cells from compartments. For example, detection may occur inside the compartment via measurements of fluorescence (e.g., direct measurement, fluorescent anisotropy, or fluorescent quenching), or DNA sequence barcode labeling/insertion. In another example, detection and sorting may occur outside of the compartments after the cells are recovered (e.g., via fluorescence, magnetic sorting, or DNA sequencing).

In another aspect a kit is described that can be used to facilitate the methods described herein. The kit may include a vector (e.g., a nucleic acid sequence) encoding, or a cell containing the nucleic acid sequence encoding, a secreted polypeptide, or an antibody, or a polypeptide containing an antibody, VH, or nanobody gene fragment. The kit may further include a vector (e.g., a nucleic acid sequence) encoding, or a cell containing the nucleic acid sequence encoding, a membrane-bound target, wherein the secreted polypeptide or antibody may be screened for binding to the membrane-bound target. The kit may also include one or more of a detection agent (e.g., for detecting the secreted polypeptide), which may include an antibody Fc effector protein detection agent, a complement protein detection agent, a ligand, a polypeptide, a chemical, a nucleic acid sequence, an antibody or fragment thereof, or a complement factor. In some embodiments, the vector encoding the secreted polypeptide and the vector encoding the membrane-bound target are incorporated within one DNA or RNA construct (e.g., a bi-cistronic vector).

In some embodiments, the target, such as a chemical moiety, a polymer, an oligomer, a peptide, a polypeptide, or a protein, is added to the cell surface or incorporate within the cell membrane before the cell is compartmentalized within the compartment. For example, cells may be exposed to a phospholipid of interest, for which a fraction of the phospholipid integrates into the cell membrane. These cells may then be compartmentalized, and the binding between the phospholipid and the secreted polypeptide and/or antibody may be detected via the methods described herein. Furthermore, the secreted polypeptide bound to the phospholipid may be tested for binding to Fc effector proteins and/or complement proteins. In other embodiments, the target may be bound to the cell surface by other covalent or non-covalent means, such as biotin-based conjugation, sortase enzymatic reactions, transpeptidase reactions, antibody binding, nanobody binding, VHH binding, aptamer binding, SpyTag, click chemistry, or other methods of attachment known to those skilled in the art.

It should be understood that the target may be added onto the cell surface/cell membrane before, during, or after compartmentalization. For example, a chemical moiety may be added at one or more points during compartmentalization, where it is able to incorporate into the cell membrane, the incorporation into the cell membrane dependent on the ability of the chemical moiety to interact with the cell membrane. In another example, a polypeptide expressed by the cell may incorporate into the membrane before, during, or after compartmentalization, depending on the ability of the cell to express the polypeptide and translocate the polypeptide to the cell membrane.

### Exemplary Embodiments

Disclosed herein are systems, kits, methods, and compositions useful for the functional screening of libraries of secreted polypeptide. In some embodiments, the systems, kits, methods, and/or compositions comprise one or more engineered cells expressing one or more test polypeptides and capable of conditionally expressing one or more reporter molecules. embodiments described below are exemplary only and are not intended to be limiting.

In certain embodiments the disclosure relates to any of the following numbered paragraphs:

1. A screening method including: (a) detecting the presence of binding between a secreted polypeptide and a membrane-bound target, wherein the secreted polypeptide and the membrane-bound target are included within a compartment, wherein the compartment includes a single, isolated genetically engineered cell, wherein the cell is engineered to secrete the secreted polypeptide.

2. A screening method including: (a) detecting a binding of an antibody Fc effector protein to a secreted polypeptide bound to a membrane-bound target on the surface of a genetically engineered cell, wherein the cell presents the membrane-bound target, wherein the cell is engineered to secrete the secreted polypeptide, wherein the cell is included within a compartment, wherein the secreted protein binds the membrane-bound target and the Fc effector protein.

3. A screening method including: (a) detecting a binding of a complement protein to a secreted polypeptide bound to a membrane-bound target on the surface of a genetically engineered cell, wherein the cell presents the membrane-bound polypeptide, wherein the cell is engineered to secrete the secreted polypeptide wherein the cell is included within a compartment, and wherein the secreted protein binds the membrane-bound target and a complement protein.

4. A screening method including: (a) detecting the presence of binding between a secreted polypeptide and a membrane-bound target on a surface of a single, isolated, genetically engineered cell, wherein the cell presents the membrane-bound target, wherein the cell is engineered to (i) secrete the secreted polypeptide; and/or (ii) express the membrane-bound target, and wherein the cell is included within a compartment.

5. A screening method including: (a) detecting a binding of an antibody Fc effector protein to a secreted polypeptide bound to a membrane-bound target on the surface of a genetically engineered cell, wherein the cell presents the membrane-bound target, wherein the cell is engineered to (i) secrete the secreted polypeptide; and/or (ii) express the membrane-bound target, and wherein the cell is included within a compartment, wherein the secreted protein binds the membrane-bound target and the Fc effector protein.

6. A screening method including: (a) detecting a binding of a complement protein to a secreted polypeptide bound to a membrane-bound target on the surface of a genetically engineered cell, wherein the cell presents the membrane-bound polypeptide, wherein the cell is engineered to (i) secrete the secreted polypeptide; and/or (ii) express the membrane-bound target, wherein the cell is included within a compartment, and wherein the secreted protein binds the membrane-bound target and a complement protein.

7. The method of any one of paragraphs 1-6, wherein the secreted polypeptide or the membrane-bound target contains a polypeptide sequence derived from an antibody, scFv, chimeric antigen receptor (CAR), VHH, or nanobody gene.

8. The method of any one of paragraphs 1-6, wherein the compartment includes a well, a droplet, spatially separated cell culture condition, or an encapsulation.

9. The method of any one of paragraphs 1-6, wherein the secreted polypeptide bound to the membrane is bound to at least one secreted polypeptide detection agent.

10. The method of paragraphs 2 or 5, wherein the wherein an Fc effector protein binding is detected by binding an antibody Fc effector protein detection agent bound to the secreted polypeptide.

11. The method of paragraphs 3 or 6, wherein complement protein binding is detected by binding of a complement detection probe to the secreted polypeptide.

12. The method of any one of paragraphs 7-11, wherein a cell bound to the secreted polypeptide detection agent, the antibody Fc effector protein detection agent, or the complement detection agent is selected by sorting.

13. The method of paragraph any one of the previous paragraphs, wherein a DNA sequence corresponding to the gene coding for at least one of the secreted polypeptide or the membrane-bound target in a selected cell is sequenced.

14. The method of any one of paragraphs 1-6, wherein the membrane-bound target includes a non-polypeptide target.

15. The method of any one of paragraphs 1-6, wherein the secreted-polypeptide or the membrane-bound target includes an antibody, a VHH, a CAR, a nanobody, or an antigen binding fragment of a heavy chain of an antibody, or a gene fragment of an antibody or nanobody.

16. The method of any one of paragraphs 1-6 wherein the cell endogenously expresses the secreted polypeptide or the membrane-bound target.

17. The method of any of one of paragraphs 1-6, wherein the cell is engineered to express the secreted polypeptide and the membrane-bound target.

18. The method of any one of paragraphs 1-6, wherein the method includes generating, before or contemporaneous with the detection step, a collection of genetically engineered cells, wherein each of genetically engineered cells 3 a gene encoding a secreted polypeptide of a library of secreted polypeptides.

19. The method of any one of paragraphs 1-6, wherein the method includes generating, before or contemporaneous with the detection step, a collection of genetically engineered cells, wherein each of the genetically engineered cells includes a gene encoding a membrane-bound target of a library of membrane-bound targets.

20. The method of any one of paragraphs 1-6, wherein the method includes generating, before or contemporaneous with the detection step, a collection of genetically engineered cells, wherein each of the genetically engineered cells includes a gene encoding a membrane-bound polypeptide of a library of membrane-bound polypeptides and a gene encoding a secreted polypeptide of a library of secreted polypeptides.

21. The method of paragraph 12, wherein at least two of the secreted polypeptide detection agent, the antibody effector protein detection agent, or the complement detection agent are detected in parallel.

22. The method of paragraph 10, wherein the antibody effector protein detection agent includes FcγRI, FcγRIIa, FcγRIIb, FcγRIIc, FcγRIIIa, FcγRIIIb, FcαRI, FcμR, FcαR, FcμR, FcεRI, FcεRII/CD23, DC-SIGN, Fcα/R, FcRn, or other Fc effector proteins, or a partial polypeptide sequence thereof.

23. The method of paragraph 10, wherein the antibody effector protein detection agent includes at least one polypeptide sequence derived from at least of one of: FcγRI, FcγRIIa, FcγRIIb, FcγRIIc, FcγRIIIa, FcγRIIIb, FcαRI, FcμR, FcαR, FcμR, FcεRI, FcεRII/CD23, DC-SIGN, Fcα/μR, FcRn, or other Fc effector binding protein genes.

24. The method of paragraph 11, wherein the complement detection agent includes at least one of C1q, C1s, C1r, a C1 complex, C1 complex proteins, C2b, C4a, C4b, C3, C3a, C3b, C5, C5a, C5b, C6, C7, C8, C9, Bb, Ba, Factor D, Factor B, Factor I, Factor H, and complement pathway protein fragments, binding partners, factors, enzymes, and proteases, or a partial polypeptide sequence thereof

25. The method of paragraph 10, wherein the detection agent includes another cell, virus, phage, or lentivirus.

26. The method of any one of the previous paragraphs, wherein the secreted polypeptide includes a T cell receptor.

27. The method of any one of paragraph 1-26, wherein the membrane-bound target includes a T cell receptor.

28. The method of any one of the previous paragraphs, wherein the secreted test polypeptide includes a peptide:MHC complex.

30. The method of any one of paragraphs 10-27, wherein the membrane-bound target includes a peptide:MHC complex.

31. The method of any one of the previous paragraphs, wherein detecting a binding and detecting a presence of binding includes detecting the presence or absence of an internalization signal.

32. The method of any one of paragraphs 1-31, wherein detecting a binding and detecting a presence of binding includes detecting the presence or absence of a protein degradation signal.

33. The method of paragraphs 32, wherein the protein degradation signal is dependent upon a proteolysis targeting chimera (PROTAC).

34. The method of paragraph 33, wherein the PROTAC includes an antibody, scFv, VHH, nanobody, TCR, pMHC, Fab, IgG, ligand, or other binding polypeptide.

35. A kit including: (a) a vector encoding a secreted polypeptide; and (b) a vector encoding a membrane-bound target, wherein the secreted polypeptide may be screened for binding to the membrane-bound target.

36. The kit of paragraph 35, further including a secreted polypeptide detection agent.

37. The kit of any one of paragraphs 35-36 further including an antibody effector protein detection agent.

38. The kit of any one of paragraphs 35-37, further including a complement detection agent.

39. The kit of any one of paragraphs 35-38, wherein the vector encoding the secreted polypeptide and the vector encoding the membrane-bound target are incorporated within the same DNA construct.

40. A screening method including: (a) detecting the presence of binding between a secreted polypeptide and a target, wherein the secreted polypeptide and the target are compartmentalized within a compartment, wherein the compartment includes a cell, wherein the target is bound to a surface of the cell, wherein the cell is engineered to secrete the secreted polypeptide, wherein the secreted protein binds the target.

41. A screening method including: (a) detecting a binding of an antibody Fc effector protein to a secreted polypeptide bound to a target on the surface of a cell, wherein the cell is disposed within a compartment, wherein the target is bound to a surface of the cell, wherein the cell is engineered to secrete the secreted polypeptide, wherein the secreted polypeptide binds the target and the Fc effector protein.

42. A screening method including: (a) detecting a binding of a complement protein to a secreted polypeptide bound to a target on the surface of a cell, wherein the cell is disposed within a compartment, wherein the target is bound to a surface of the cell, wherein the cell is engineered to secrete the secreted polypeptide, wherein the secreted polypeptide binds the target and a complement protein.

43. The method of any one of paragraphs 1 to 34 or 40 to 42, wherein the target includes at least one of a chemical moiety, a nucleic acid, a peptide, a polypeptide, or a protein.

44. The method of any one of paragraphs 1 to 34 or 40 to 43, wherein the target is bound to or expressed on the cell surface prior to compartmentation.

45. The method of any one of paragraphs 1 to 634 or 40 to 44, wherein the target is bound to or expressed on the cell surface during or after compartmentation.

46. The method of any one of the previous paragraphs, where a DNA-barcoded moiety is associated with the membrane-bound target or the secreted polypeptide.

47. The kit of any of paragraphs 35-39, wherein a DNA-barcoded moiety is associated with the membrane-bound target or the secreted polypeptide.

### EXAMPLES

The following Examples are illustrative and should not be interpreted to limit the scope of the claimed subject matter.

### Example 1: Establishment of a Cell Line for Concurrent mAb Secretion and Viral Infection

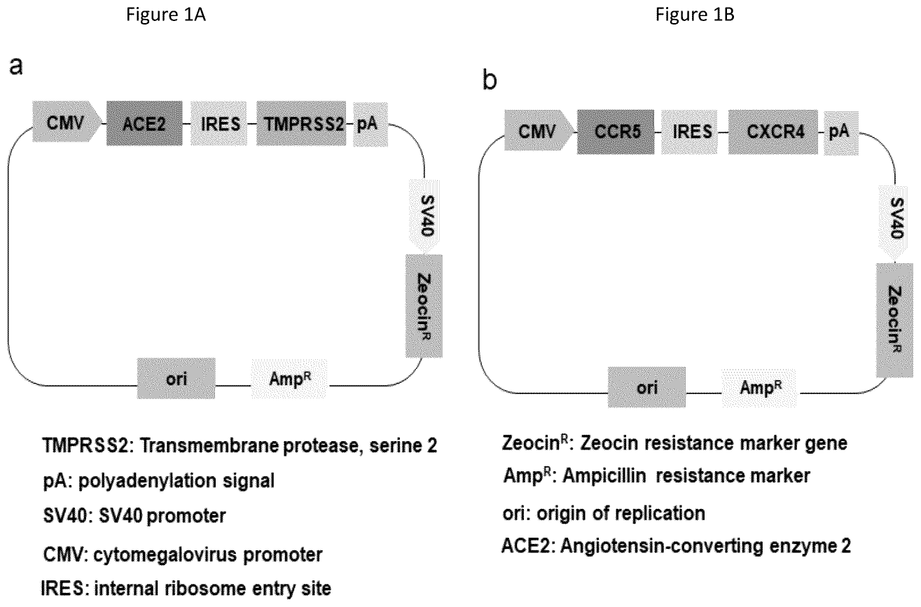

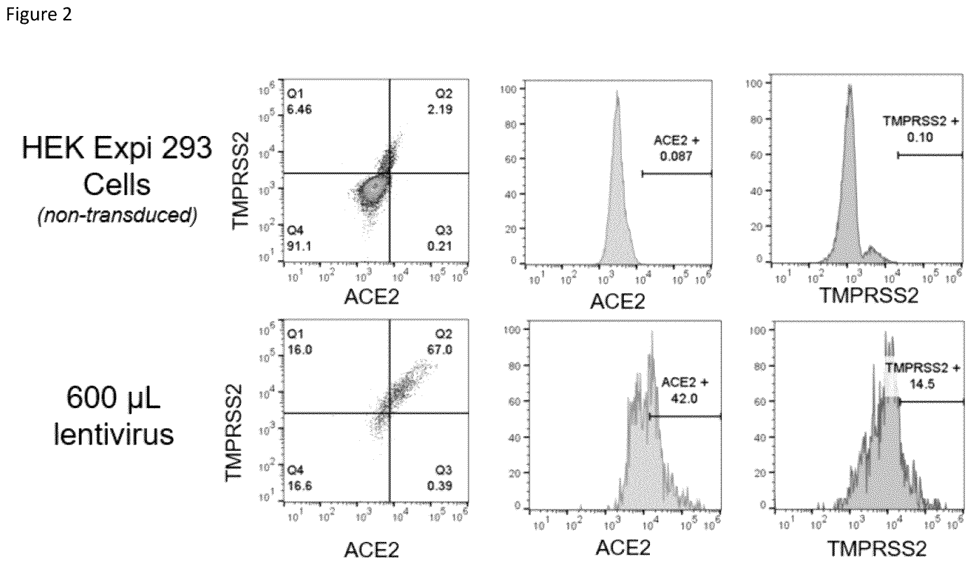

In this working example, SARS-CoV-2 receptor/co-receptors and anti-SARS-CoV-2 antibody were used as an example application of a neutralization assay performed with the same cell line for both protein secretion and viral infection concurrently. A mammalian cell line was developed, expressing anti-viral antibodies and their respective viral entry receptors or co-receptors to permit viral infection concurrently with antibody secretion. As an example virus application, an anti-SARS-CoV-2 antibody and its receptor human Angiotensin-converting enzyme 2 (hACE2) and/or Transmembrane Serine Protease 2 (TMPRSS2) were expressed in a mammalian cell line (FIG. 1). A bi-cistronic vector was constructed containing a human cytomegalovirus promoter, the ACE2 surface receptor of SARS-CoV-2 infection, an internal ribosome entry site (IRES), and the TMPRSS2 gene, allowing co-expressing hACE2 and TMPRSS2 in a mammalian expression vector. (The TMPRSS2 gene is optional and is not required for SARS-CoV-2 infection but can enhance the ability of virus to infect some cells⁸). This expression cassette was cloned into a vector with a selectable marker for plasmid transfection, enabling transformed cell selection using the selectable marker. We transfected this plasmid into HEK293 cells by mixing the plasmid with lipofectamine transfection reagents. Two days later, the cell culture media was replenished, and the selection reagent respective to the selectable marker was added to the culture media for initiating the selection process. After 7-14 days, we obtained a stable cell pool, which was resistant to the selection reagent. Then this pool of cells was stained with both fluorescent-conjugated anti-ACE2 and fluorescent-conjugated anti-TMPRSS2 (FIG. 2). Cells were sorted for ACE2 and TMPRSS2 expression and the cells were rested for recovery. After the cell recovery with regular growth, limiting dilution cloning was performed to isolate a single clone. After 10-15 days, the single clone formed a cell colony which was transferred into 24 well plates allowing the cells to expand. After the cell expansion, we stained the clones with antiACE2 and anti-TMPRSS2 and selected the clone with highest ACE2 and TMPRSS2 expression (Named, HEKACE2/TMPRSS2).

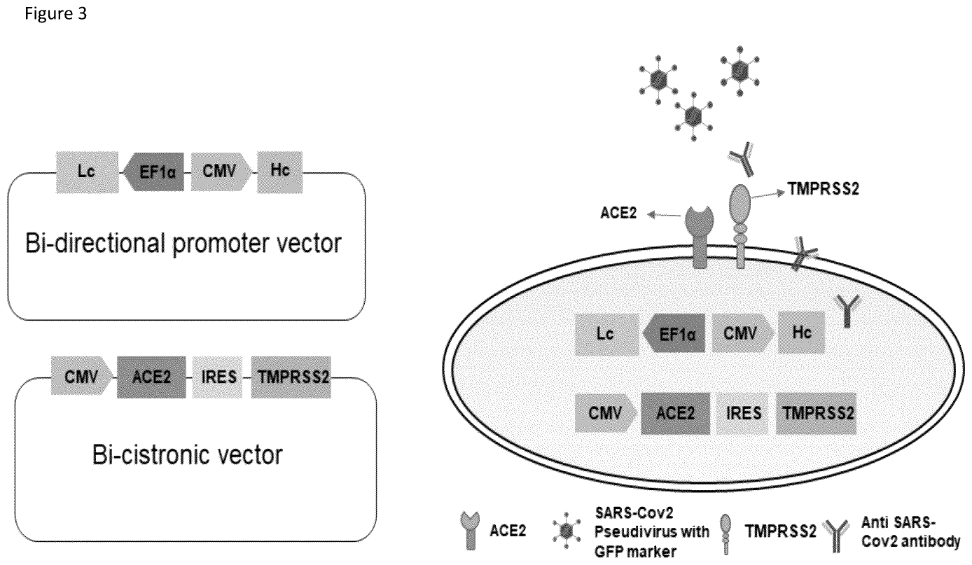

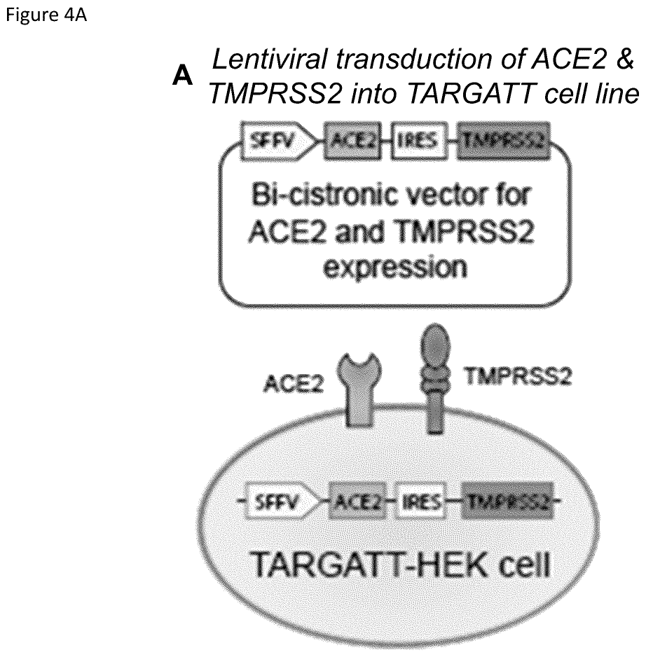

We then transformed these cells to express complete IgG of antibodies with known SARS-CoV-2 neutralization capacity. Alternatively, we could express other IgG fragments such as single-chain variable fragment (Scfv), antigen-binding fragment (Fab), or bi-specific antibody. We cloned the desired antibody or antibody fragment into a mammalian vector with a selectable marker. Followed by transfection and selection with selection marker reagent, the IgG expressing HEKACE2/TMPRSS2 stable pool was then subjected to limiting dilution cloning to isolate the individual secreted protein expressing clone, which in this case was an antibody IgG. After 10-15 days of cell expansion, we transferred 50 μL of cell culture media to evaluate the IgG expression by direct ELISA. The ELISA was performed by coating the IgG overnight in 96 well plates. The plates were washed with Phosphate-buffered saline with 0.05% Tween 20 (PBST) and were blocked with 5% BSA in PBST for 2 hours. We washed the plates three times with PBST and added the HRP-conjugated rabbit anti-human Fc antibodies onto the well and incubated for two hours. The plates were washed with PBST four times and the 3,3′,5,5′-Tetramethylbenzidine Liquid Substrate was added for HRP reaction and stopped with 2M H₂SO₄ for detection. We analyzed the plates using a plate reader at the absorbance wavelength of 450 nm. We then compared the absorbance of the IgG expressed by HEKACE2/TMPRSS2 stable clones with IgG standard to estimate the relative IgG expression yield. We selected the highest IgG expressing clone as our candidate clone. We generated a cell line include ACE2, TMPRSS2 and IgG allowing a neutralization assay to be performed in a single cell (FIG. 3). Several strategies for cell line development can be used (FIG. 4).

### Example 2: Quantification of Infection Against Viruses or Pseudoviruses in a Modified Mammalian Cell Line Capable of Soluble Protein Secretion

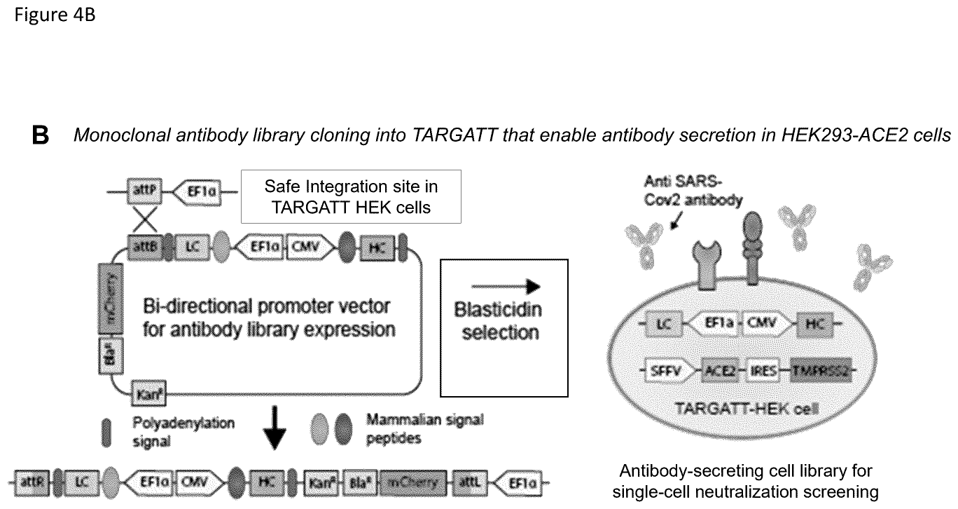

The HEK293 cell line can be used for protein expression or secretion in lab experiments. In this working example, we used a HEK293 cell line with ACE2 expression but no TMPRSS2 expression for the pseudovirus neutralization assay (Named HEKACE2). We infected a HEKACE2 with a strain of lentiviral-based pseudovirus encoding SARS-CoV-2CoV2 spike protein on the viral surface with a GFP reporter gene in the viral expression vector; SARS-CoV-2 pseudovirus infected cells would thus express GFP. We detached the HEKACE2 cells with 0.05% trypsin and stopped the trypsin reaction with DMEM media with 10% FBS. We then counted the cell density and resuspended cells to a density of 3×10⁵ cell/mL and added 100 μL of the cell suspension to each well in the 96 well plate. We retrieved an aliquot of frozen pseudovirus and added various amount of the virus (15 μL, 30 μL, 60 μL and 90 μL) to the 96 wells. The 96-well plates were incubated at 37° C. for 48-72 hours before neutralization was quantified by acquiring GFP signal using flow cytometry. As indicated in FIG. 5, the percentage of the pseudovirus infected HEKACE2 cells, reflected by the percentage GFP positive cells, correlated to the amount of virus added (FIG. 5).

### Example 3: Enabling Protein Secretion in Single Cells, with a Library of Encoded Protein Variants

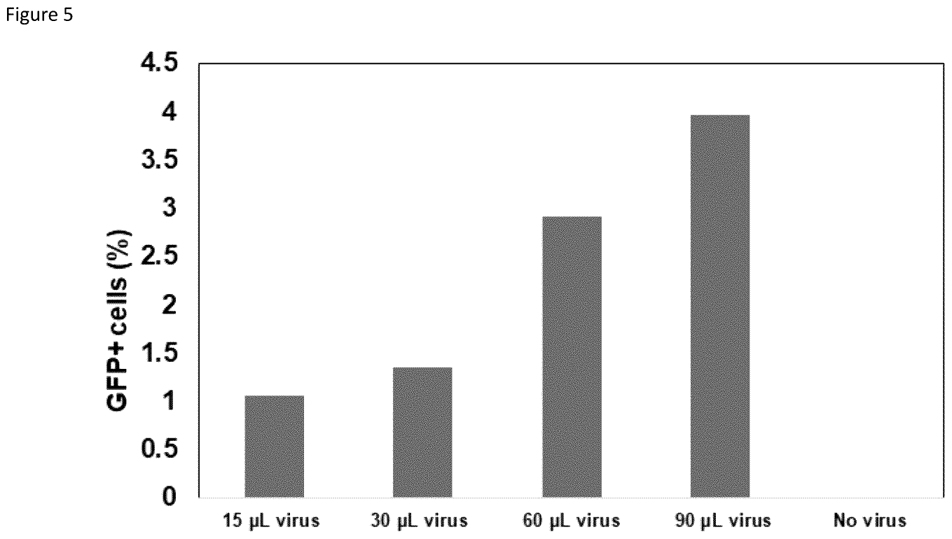

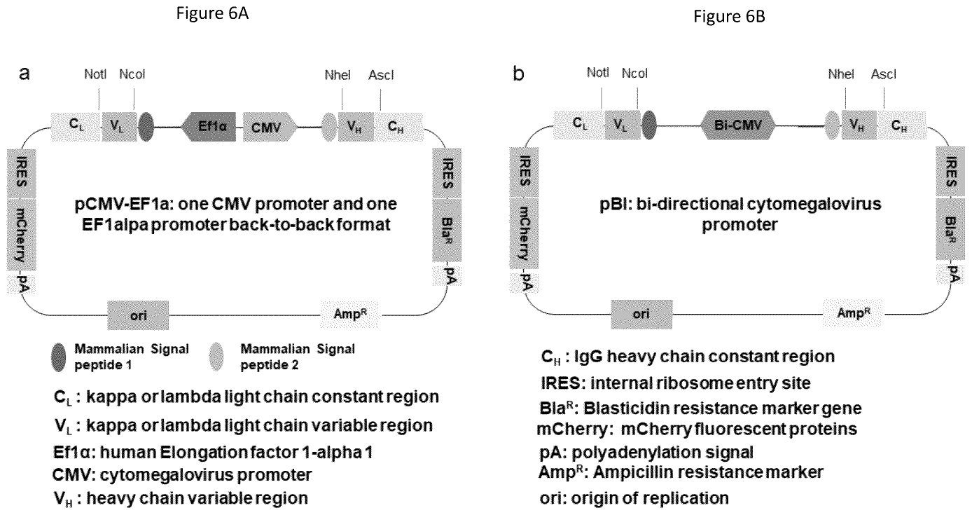

In this working example, we show methods to enable protein secretion in single cells for subsequent protein secretion and assay-based selection. In this example, the secreted protein is an antibody IgG. We can obtain libraries of natively paired antibody heavy and light chain variable regions (VH:VL) from patients having a SARS-CoV-2 infection as described in the protocol in McDaniel et al ⁽³⁾. We cloned paired VH:VL sequences into a plasmid vector with one CMV promoter and one EF1alpa promoter (pCMV-EF1a, FIG. 6) or a vector with a bi-directional promoter (FIG. 6, pBI), similar to a format previously described^{(10,11,12,13,14)}. The cloning of a VH:VL library into the pCMV-EF1a and pBI utilize NotI and NheI cutting sites to clone the amplicon into the backbone vectors without the promoter and we then cloned in the dual promoters (CMV and EF1a) or the bi-directional vector (Bi-CMV) using NheI and NcoI site on the leader peptide region of the heavy chain and light chain, respectively. Prior studies have shown that changing a protein's leader peptide can modulate the level of protein expression^{10,11,12,13,14)}. We designed different leader peptides to achieve varied levels of protein secretion. We designed three leader peptides that included an NheI cut site for the heavy chain (Le11, Alb2 and L2B) and two leader peptides that included a NcoI cut site for the light chain (Le12 and Alb1) (Table 1). We expressed two antibodies (anti-HIV antibody VRCO1 and anti-SARS-CoV antibody, CR3022) with six different heavy chain and light chain leader peptide combinations (Table 2) in two different vectors (pBI or pCMV-EF1a). We transfected these 24 IgG constructs into HEKACE2 cells (without TMPRSS2 expression) using lipofectamine. Three days post transfection, we detected the IgG expression via ELISA as described in Example 1. As shown in FIG. 6, for both VRCO1 and CR3022 antibodies, we observed that all leader peptide combinations enable IgG expression, and different leader peptide combinations could be selected for modulation of the concentrations of secreted antibody desired. With LP4 and LP5 we observed the highest two IgG expressions in the pCMV-EF1a vector (FIG. 7). We generated a stable IgG expressing pool by adding Blasticidin to a final concentration of 5 μg/mL.

TABLE 1
Leader peptide amino acid sequences and DNA sequences

| Light Chain | Light chain leader | |
|---|---|---|
| leader | peptide amino acid | |
| peptide | sequence | DNA sequence |
| Lell | MSKRGASNEMALLL | ATGAGCAAGAGGGGCGCCAGCAACGAGAT |
| | LLLGLLQQAMA | GGCCCTGCTGCTGCTGCTGCTGGGCCTGCT |
| | (SEQ ID NO: 17) | GCAGCAGgccatggcg (SEQ ID NO: 18) |
| Alb2 | MKWVTFISLLFLESS | ATGAAGTGGGTGACCTTCATCAGCCTGC |
| | AMA (SEQ ID NO: 19) | TGTTCCTGTTCAGCAGCgccatggeg (SEQ ID |
| | | NO: 20) |
| L2B | MKYLLPTAAAGLLL | ATGAAGTATTTGTTGCCGACGGCGGCG |
| | LAAQPAMA | GCGGGGTTGTTGTTGTTGGCGGCGCAG |
| | SEQ ID NO: 33) | CCGgccatggcg (SEQ ID NO: 34) |
| Heavy chain | Heavy Chain leader | |
| leader | peptide amino acid | |
| peptide | sequence | DNA sequence |
| Le12 | MSKRGASNEMALLL | ATGAGCAAGAGGGGCGCCAGCAACGAGAT |
| | LLLGLLASVLA (SEQ | GGCCCTGCTGCTGCTGCTGCTGGGCCTGCT |
| | ID NO: 21) | Ggdagcgttttagca (SEQ ID NO: 22) |
| Alb1 | MKWVTFISLLELFAS | ATGAAGTGGGTGACCTTCATCTCCCTG |
| | VLA(SEQ ID NO: 23) | CTTGCCTGTTCGCTAGCGTTTTAGCA |
| | | (SEQ ID NO: 24) |

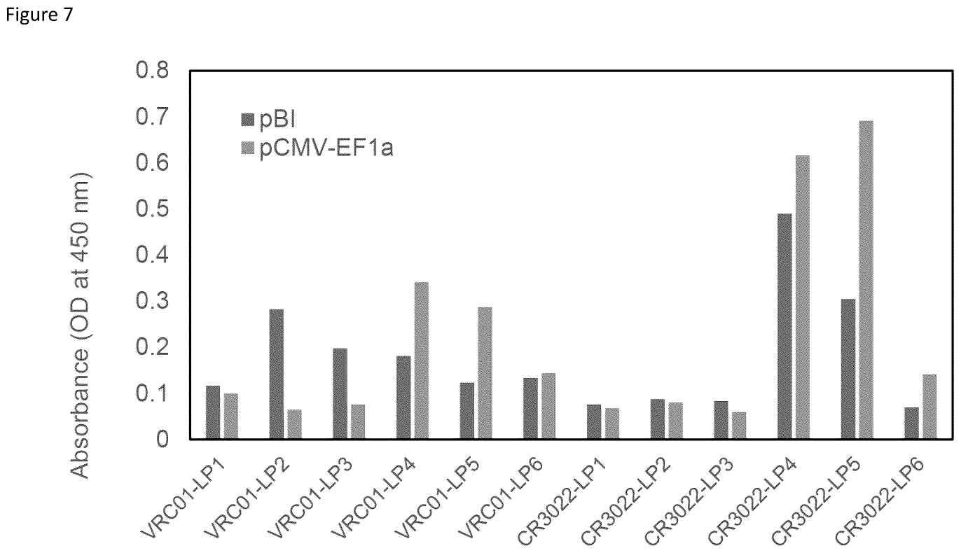

Alternatively, we could express the natively paired VH:VL into HEKACE2 derived from the Flip-In HEK293 kit via Flp recombinase-mediated integration at the FRT site (FIG. 8). To do so, we would first clone the IgG expression gene cassette into the pcDNA5/FRT vector. We would then co-transfect the engineered pcDNA5/FRT vector with Flp recombinase vector pOG44 into the HEK-Flp-In 293 with ACE2 expression. We would generate a library of IgG expressing cells by hygromycin selection. The Flp-mediated cloning has an advantage in that only a single protein variant is encoded by each cell, which is helpful for the selectivity of our assay, although not strictly necessary for assay implementation.

TABLE 2
Heavy chain and light chain leader peptide combinations

| Leader | Heavy chain | Light chain |
|---|---|---|
| peptide | leader | leader |
| pair name | peptide | peptide |
| LP1 | Alb1 | L2B |
| LP2 | Alb1 | Le11 |
| LP3 | Alb1 | Alb2 |
| LP4 | Le12 | Le11 |
| LP5 | Le12 | Alb2 |
| LP6 | Le12 | L2B |

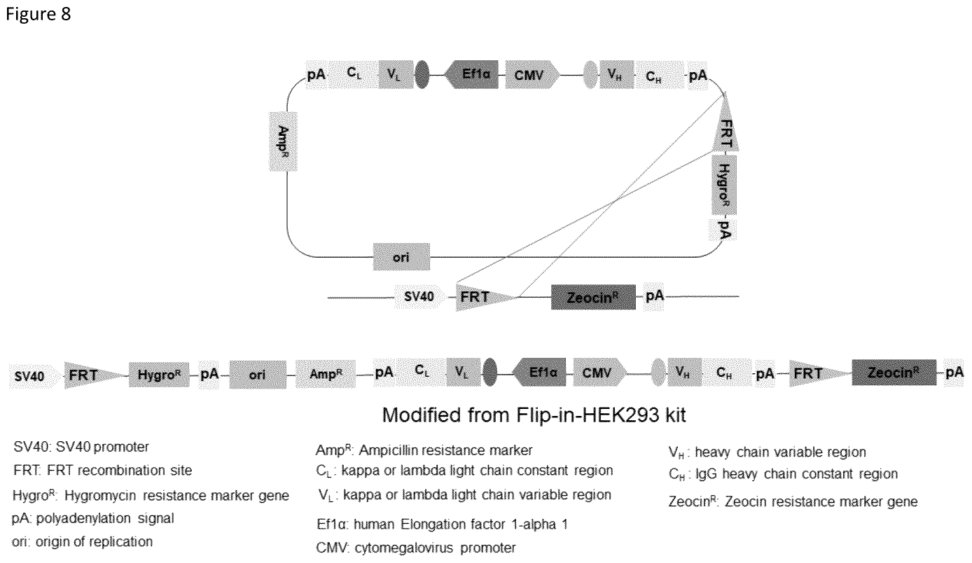

In an alternative working example, we used an integrase-based gene integration system to express IgG from HEK293ACE cells derived from the TARGATT™-HEK293 master cell lines. We cloned the IgG expression gene cassette into a donor vector containing the integrases recognition site, attB, blasticidin resistance marker and mCherry (FIG. 9). We then co-transfected the donor plasmid and integrase expression plasmid into an engineered HEK cell line stably expressing ACE2. The attP landing pad was at the hH11 gene locus

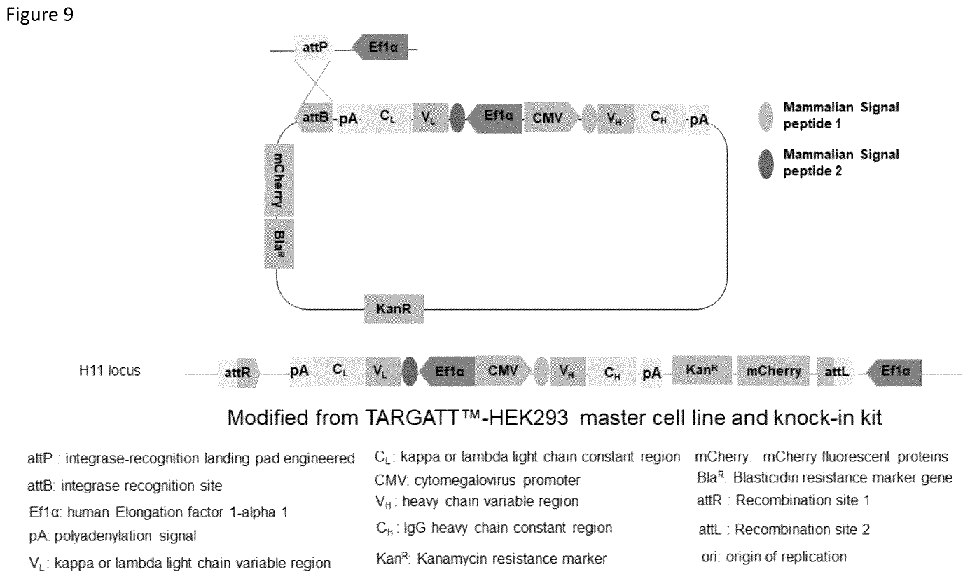

In another working example, we used the CRISPR homologous directed repair platform system to express the IgG in HEK-ACE2 cells. We co-transfected donor IgG expressing cassette (VH:VL sequences with dual promoter or bi-directional promoter) with homologous arms (FIG. 10). The gRNA/Cas9 expressing vector provided integration of the natively paired VH:VL sequence into a safe harbor gene locus. Targeted safe harbor loci include CCR5, AAVS1, and Hipp11 (FIG. 10).

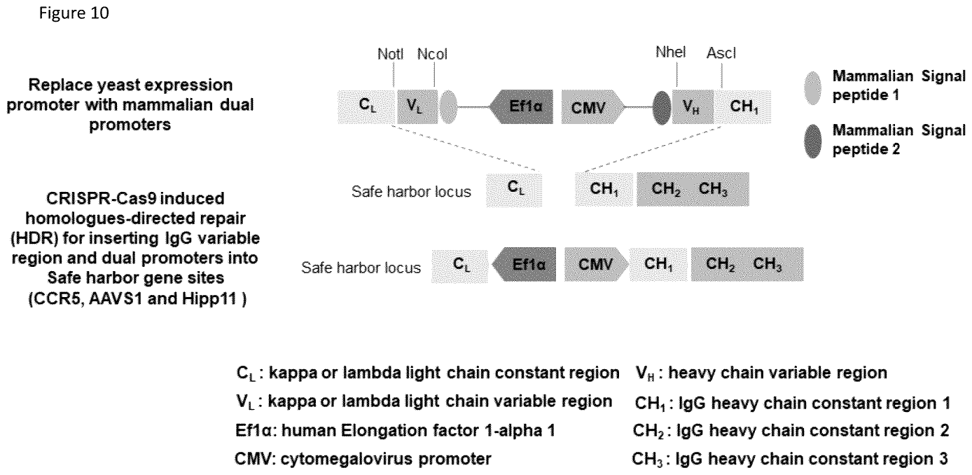

The cloning and transformation methods can be suitably matched to the cell lines and cell-based functional activity model of interest. Several different cloning and transformation methods can be suitably used for generating libraries of secreted proteins into mammalian or other cells (FIG. 11). Other types of cloning can be used to insert nucleic acids for protein secretion into host cells, which can include, without limitation, lentiviral gene transfer, infectious molecular clones, adenoviral vectors, adeno-associated viral vectors, chemical DNA transfection, chemical RNA transfection, mRNA encapsulated by nanoparticles, DNA encapsulated by nanoparticles, or other methods known to the art to induce cell expression of desired proteins and plasmid vectors.

### Example 4: The Generation of Antibody Protein Libraries for Cloning and Soluble Antibody Functional Analysis in VH:VL Bidirectional Format

In this prophetic example, randomly paired VH:VL libraries or mutational VH:VL libraries can be synthesized via a gene synthesis service, where the VH and VL genes of the antibody are linked by a DNA linker. Alternatively, VH:VL gene libraries can be amplified directly from human, mouse, or non-human primate samples, as reported previously⁽¹⁵⁾. In some embodiments, we can introduce diversity into the libraries using error-prone PCR, site-saturation mutagenesis, and/or DNA shuffling. We can clone the VH library by using a combination of NotI and NcoI and clone the VL library by using a combination of NheI and AscI in a dual promoter (back-to-back format), or we can maintain the bi-directional promoter format as illustrated in FIG. 6, by using first NotI and AscI to clone the full construct, and then using NcoI and NheI to clone in the bidirectional promoter, similar to previous reports^{(16,17)}. We can transfect this plasmid into the HEKACE2 cells for IgG expression. Alternatively, we can clone the gene cassette (dual promoters or bi-directional promoter with a heavy chain and a light chain) into the FLP/FRT based gene integration donor plasmids (FIG. 8), integrase-based donor plasmids (FIG. 9), or CRISPR/Cas9 donor plasmid for stable IgG integration (FIG. 10) into a safe harbor gene locus. Several possible cloning strategies can be used for bidirectional antibody expression (FIG. 11).

### Example 5: Methods to Enable Synthetically Generated Antibody Expression in Standard One-Direction Format

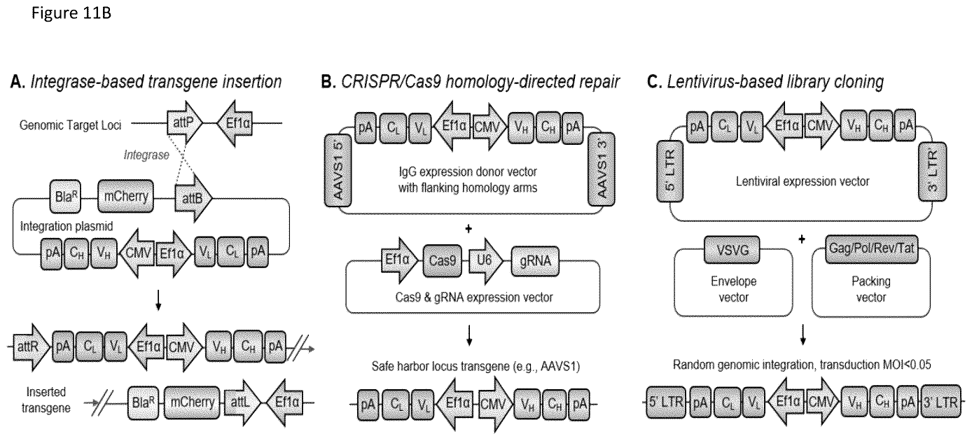

We can synthesize randomly paired VH:VL libraries or mutational VH:VL library via gene synthesis service. We can then clone the VH and VL separately into a mammalian expression vector and express the IgG in one open reading frame as illustrated in FIG. 12a.

IgG can be expressed in a single-chain variable fragment format with GS linker in between the heavy chain and light chain variable region.

Alternatively, full IgG can be expressed in a bi-cistronic format with a p2A cleavage peptide between the IgG heavy chain and the light chain (FIG. 12B) (see Yellow Fever working examples, below). We transfected the plasmid and expressed the IgG.

We can integrate the one-directional format of IgG into a safe harbor locus expression site. Options for integration include FLP/FRT based gene integration (FIG. 8), integrase-based donor plasmid (FIG. 9), transposon-based integration, or with the use of CRISPR/Cas9 donor plasmids for stable IgG integration (FIG. 10). Several cloning and transformation methods are suitable, depending on the cell line and functional model to be used for the secreted proteins (e.g., FIG. 11).

### Example 6: Application of the Secreted Protein Assay in Well Plates as a Compartment for Selection of Antibodies with Viral Neutralization Properties

In this working example, we show a functional assay using secreted protein along with a readout of secreted protein activity in the same cell lines. In this example, the secreted protein is an antibody, and the activity to be assayed is neutralization of SARS-CoV-2 pseudovirus, and the selection marker is GFP.

2×10⁴ of HEKACE2 cells were seeded in a 96 well plate to reach a confluency of 70% at the time of transfection. Following the protocol included with the Lipofectamine™ 3000 Reagent Kit (Invitrogen), 100 ng of mAb in mammalian expression vector pBI per well was diluted in 5 μL of Opti-MEM™ Reduced Serum Media (Thermo Fisher Scientific), and 0.2 μL of P3000™ Reagent was added. Separately, 0.2 μL of Lipofectamine™ 3000 Reagent was also diluted in 5 μL Opti-MEM™. The diluted DNA was added to the diluted Lipofectamine™ 3000 Reagent and incubated at room temperature for 12 minutes. This DNA-lipid complex was then added to the HEKACE2 cells and incubated at 37° C. for 3 days. As the pBI vector contains mCherry on the light chain of the mAb, the cells could be visualized using a fluorescence microscope or flow cytometry following transfection to confirm gene expression. Three days following transfection, the neutralization activity of the antibodies was be measured as described below.

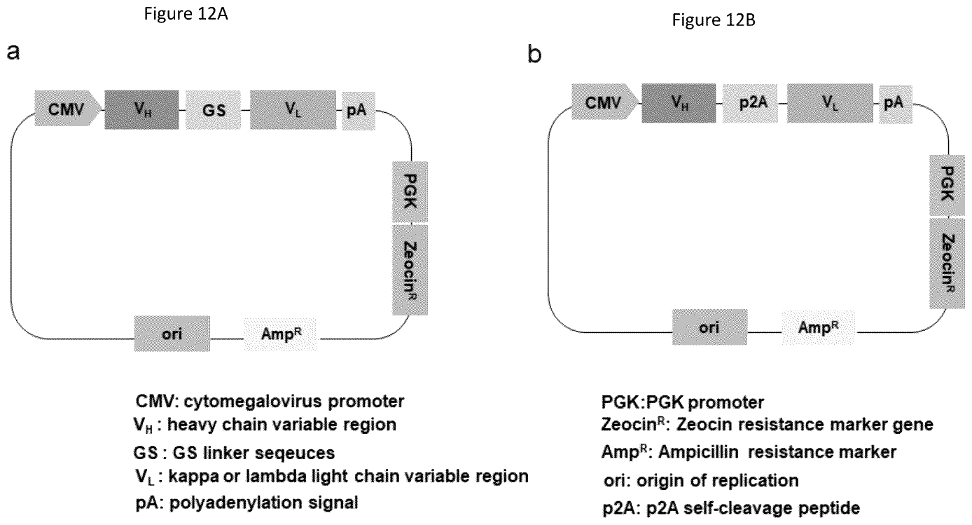

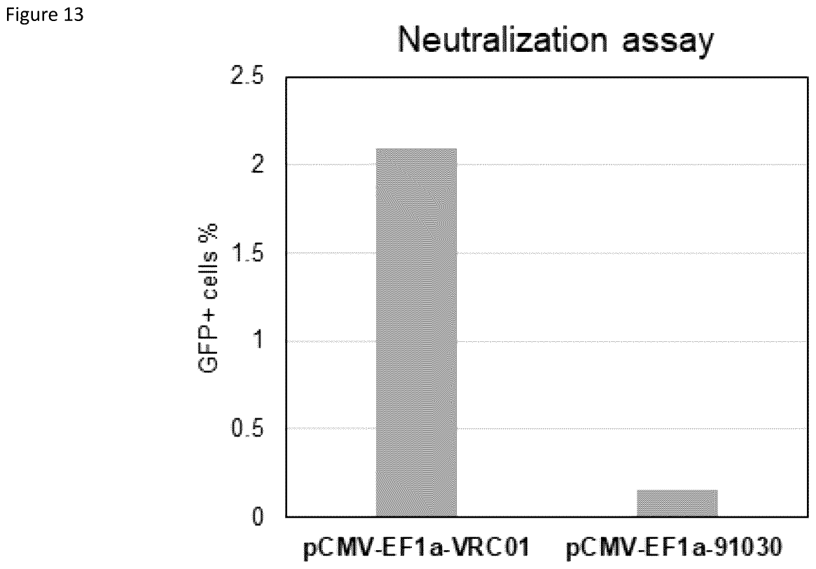

SARS-CoV-2 Wuhan Hu-1 GFP reporter virus particles (Integral Molecular) were thawed and placed on ice, 60 μL of the reporter virus particles were added directly to the cell media. The 96-well plates were incubated at 37° C. for 48-72 hours before neutralization was quantified by acquiring GFP signal using flow cytometry. ELISA analysis of IgG expression indicated that VCR01 (an anti-HIV antibody) has a higher IgG expression level than that of the antibody 910-30⁽¹⁷⁾, an anti-SARS-CoV-2 antibody. Neutralization assay showed that HEKACE2 expressing VCR01 exhibited a higher GFP population than the HEKACE2 cell expressing 910-30 (FIG. 13). Similar results were obtained when repeating the experiments (FIG. 14).

### Example 7: Selection of Neutralizing Antibodies from a Library of Antibodies Encoded by Cells Capable of Both Antibody Secretion and Pseudovirus Infection

In this prophetic example, we generate a mixture of the stable cell lines expressing secreted VRC01 and 910-30, as described in Example 6. We perform limiting dilution isolation to generate single cells in a 96-well plate, with an average of 0.25 cells per well. Cells are permitted to expand for 40 days after the limiting dilution cloning, and then we add 30 μL of pseudovirus for direct neutralization assay. We retrieve the cells and sort the GFP+ populations, which are enriched for cells that were not protected from infection by the antibodies they secrete (i.e., were expressing non-neutralizing antibodies). We also sort the GFP-population to enrich for cells protected from infection (i.e., expressing neutralizing antibodies.) We retrieve the paired antibody DNA gene sequences from GFP- and GFP+ populations by extracting the RNA and performing RT-PCR for the antibody genes⁽²²⁾. We perform high-throughput sequencing to obtain the VH:VL information in the GFP- and GFP+ populations. We compare the frequency of antibody variants in each population and determine the neutralization capacity of each antibody in the population⁽²¹⁾ pool. We find that VRCO1 sequences were comparatively enriched in the GFP+ virus-infected group, whereas 910-30 sequences were comparatively enriched in the GFP-group, and these quantitative signals of selection demonstrate the ability of our secreted protein assays to test for the virus neutralization capacity of encoded antibodies secreted by cells.

To discover natively paired VH:VL antibodies directly from B cells, we clone the native VH:VL library from SARS-CoV-2 human patient samples into the IgG expressing vector and express in HEKACE2 cells as described in Example 3, Example 4 and Example 5. We perform limiting dilution cloning isolate the single cell in a 96 well with one cell per well. Forty days after the limiting dilution cloning, we add the 30 μL of pseudovirus for direct neutralization assay. We sort the GFP+ population, which was enriched for non-neutralizing antibodies. We sort the GFP-population to enrich the population for neutralizing antibodies. We retrieve the paired antibody DNA gene sequences from GFP- and GFP+ populations by extracting the RNA and performing RT-PCR for the antibody genes⁽²²⁾. We perform high-throughput sequencing analysis to obtain the VH:VL information. We compare the frequency of antibody variants in each population to determine the identify of neutralizing antibodies in the population⁽²¹⁾.

### Example 8: Application of Secreted Protein Functional Assays in Printed Microchambers as a Compartment

In this prophetic example, we first generate the paired VH:VL expressing HEKACE2 cells by one of the methods described in Example 3, Example 4, and Example 5. A population of cells is added into 125-pl wells molded in polydimethylsiloxane (PDMS) slides⁽²⁾. Each slide contains 1.7×10⁵ wells; we process four slides simultaneously to include 68,000 IgG expressing HEKACE2/TMPRSS2 cells at an approximately 1:10 cell-to-well ratio occupancy, enabling a greater than 95% probability of single-cell per well according to Poisson statistics. We incubate the slide at 37° C. 5% CO₂ incubator for overnight, allowing IgG secretion. SARS-CoV-2 pseudovirus is deposited over the microwells to diffuse inside and the PDMS slides are sealed with a dialysis membrane. We incubate the slides for 16 hours allowing the virus entry to the cells. The slides are washed, and the live cells are recovered from the slides in the presence of high concentrations (1 mg/mL) of soluble 910-30 neutralizing IgG to prevent subsequent viral infection once cells are pooled together. The cells are seeded into a 24 well plate to recovery and expand at 37° C. 5% CO₂ incubator for two days. We centrifuge the cell and resuspend in FACS buffer. We recover GFP- and GFP+ populations and extract the RNA and performing RT-PCR for the antibody genes⁽²²⁾. We perform high-throughput sequencing analysis to obtain the VH:VL information. We compare the frequency of antibody variants in each population to determine the identify of neutralizing antibodies in the population^{(3,21)} as described in Example 6.

Alternatively, we screen the anti-SARS-CoV-2 neutralizing antibody via Lightning Optofluidic System. We load the IgG expressing HEKACE2/TMPRSS2 cells onto the OptoSelect™ chip with NanoPen™ chamber to isolate them in a one-cell-per-chamber basis and incubate overnight for cells to secret antibody. The SARS-CoV-2 pseudovirus is added to the chambers and incubate for three days. The live cells are recovered from the Nanopens in the presence of high concentrations (1 mg/mL) of soluble 910-30 neutralizing IgG to prevent subsequent viral infection once cells are recovered together. We isolate GFP- and GFP+ populations using fluorescence activated cell sorting (FACS) and extract the RNA and perform RT-PCR for the antibody genes⁽²²⁾. We perform high-throughput sequencing analysis to obtain the VH:VL information. We compare the frequency of antibody variants in each population to determine the identify of neutralizing antibodies in the population^{(3,21)} as described in Example 6.

### Example 9: Application of the Assay in Emulsion Droplet Systems

In this working example, we used a microfluidic device to encapsulate the IgG expressing HEKACE2 cells in cellular secretion media to form droplets with one cell per droplet^{(6,25,26)}. An example of cell isolation and antibody secretion using CHO cells transiently transfected for antibody secretion is shown in FIG. 15.

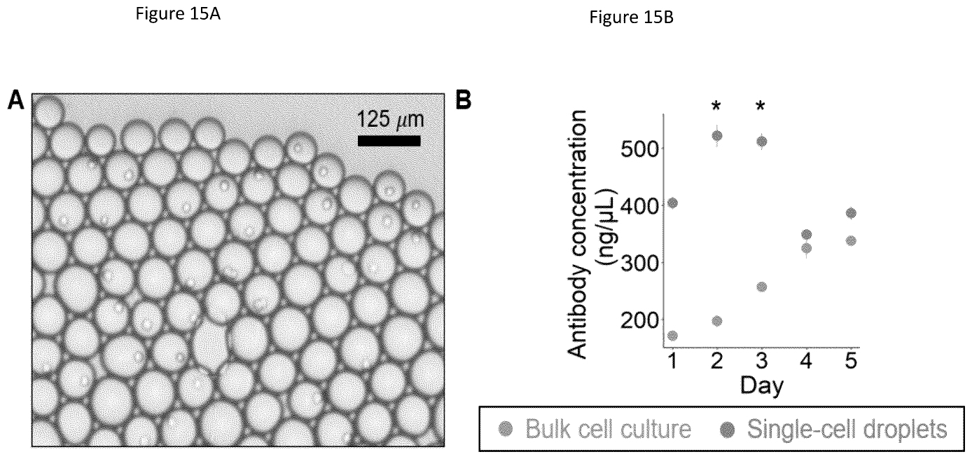

We implemented this system for SARS-CoV-2 secreted protein neutralization assays using HEKACE2 cells. The workflow for neutralization assays using cells secreting proteins inside emulsion droplets is shown in FIG. 16. We incubated the droplet for between 4 and 96 hours, although a broader time scale can also be used, allowing the secretion of the secreted protein (in this example, IgG) within the droplet. Subsequently, a second droplet containing SARS-CoV-2 pseudovirus was merged into the droplet with IgG expressing HEKACE2 cells. We used electrocoalescence to merge droplets⁽²⁴⁾, although alternative droplet merging methods are also known to persons skilled in the art and include micropillar resistance arrays. The merged droplets were further incubated for another 4-96 hours to allow pseudovirus infection or neutralization to occur, although a broader time scale can also be used. The droplets were broken, and the cells were recovered.

Droplets can be broken using chemical reagents, including 1H,1H,2H,2H-Perfluoro-1-octanol, or other methods known to individuals skilled in the art. Optionally, a potently neutralizing compound (for example, a high concentration of neutralizing antibody) can be added to the system to prevent any new pseudovirus infections after the droplets are merged together. As one salient example, the droplets are broken, and cells are recovered from droplets in the presence of high concentrations (1 mg/mL) of soluble 910-30 neutralizing IgG to prevent subsequent viral infection once cells were recovered together. Optionally, the recovered cells can be cultured for additional hours, days, weeks, or months prior to screening. Next, we isolated GFP- and GFP+ cell populations using fluorescence activated cell sorting (FACS) and extracted the RNA and performed RT-PCR for the antibody genes⁽²²⁾. We will perform high-throughput sequencing analysis to obtain the VH:VL information in the GFP- and GFP+ cell groups. We will compare the frequency of antibody variants in each population to determine the identify of neutralizing antibodies in the population^{(3,21)} as described in Example 6, where GFP-cells are enriched for encoding antibodies that provide protection against SARS-COV-2 pseudovirus infection, whereas GFP+ cells are enriched for encoding antibodies that are not protective against SARS-CoV-2 or that do not express at sufficient quantities to provide protection inside droplets under the assay conditions used.

### Example 10: Neutralizing Antibody Discovery from Native Antibody Libraries^{(9,16,17,18,19,20)}

In this prophetic example, we obtain the natively paired VH:VL library using a native paired VH:VL sequencing platform. The VH:VL amplicon can be delivered as IgG or IgG fragments via random gene integration using plasmid transfection and resistance gene marker selection, as well as via site-specific integration, as described in Example 3. We screen potent SARS-CoV-2 neutralizing antibodies using multiple methods for single cell isolation, including single cell isolation into well plates (Example 7), printed chambers (Example 8), or microfluid droplets (Example 9). We then sort the GPF- and GFP+HEKACE2 cells and perform RT-PCR to obtain paired VH:VL amplicons from each cell population. We perform PCR to add a primer barcode for next-generation sequence analysis of antibody populations, as described previously^{(16,17,19)}. We choose 10 of the most prevalent VH:VL clones enriched in the GFP negative population and for gene synthesis. We then performed transient transfection of plasmids to express IgG in a suspension of Expi293 cells. 7 days after transfection, we centrifuge the culture and transfer the supernatant into 50 mL centrifuge tube. We add 0.5 mL of protein G resin and allow the reaction to carry on a bench rotator for 2 hours. We then pour the reaction mixture into the polypropylene columns to retain the protein G resin. We then elute IgG with 0.1 M glycine-HCl, pH 2.7 and neutralize the pH with 1M Tris-HCl, pH 9.0. The purified IgGs are then concentrated and subjected to neutralization assay analysis of individual IgG. We quantified the IgG protein concentration by BCA protein assay. We then analyze the purity of the IgG by mixing 2 μg of purified IgG with SDS-page sample buffer and run through the TGX Stain-Free Precast Gel. We perform serial dilution of the antibodies from 10 μg/mL to a final concentration of 0.001 μg/mL. We combine serially diluted antibodies with 30 μL SARS-CoV-2 pseudovirus and incubate the reaction for an hour at 37° C. We then add the virus-antibody mixture into a 96 well with 2×10⁵ ACE expressing HEK293 cells. We incubate at 37° C. 5% CO₂ for three days. We analyze the antibodies' neutralization activity via flow cytometry analysis of the GFP signal to demonstrate the recovery of neutralizing antibodies that are enriched in the GFP-cell population

### Example 11: Antibody Library Variant Expression and Directed Evolution Selection for Potent Neutralizing Antibodies

In this prophetic example, we mutate an anti-SARS-CoV-2 antibody by one of the methods from DNA shuffling, error prone-PCR, single-site directed mutagenesis to generate antibody variant libraries. To increase the mutational landscape, we can further perform combinatorial and/or sequential mutation. We clone the synthetic antibody mutant libraries into HEKACE2 cells described in Example 3. We screen the SARS-CoV 2 neutralizing antibodies by one of the approaches delineated in Example 7, Example 8 and Example 9. After the sorting of GFP- and GFP+ cells and subsequent recovery of the VH:VL sequences information via RT-PCR from the GFP-negative IgG-expressing HEKACE2 cells (enriched for secretion of neutralizing antibodies), we re-deliver the screened VH:VL gene into the IgG expressing vectors detailed in Example 3 (named enriched IgG libraries) for subsequent rounds of screening. We can also perform sequential mutation using DNA shuffling, error prone-PCR, single site directed mutagenesis to enhance the diversity between screening rounds; other DNA sequencing and library diversity generation strategies can also be used and are known to those skilled in the art. We express both enriched libraries and enriched plus mutated IgG libraries on an IgG expressing platform, as presented in Example 3. We re-screen and obtain the neutralizing antibodies VH:VL sequences using methods described in Example 7, Example 8 or Example 9. We repeat the re-delivery and screen of enriched libraries for subsequent rounds to further enrich for neutralization potency, until a molecule with the desired neutralization potency is obtained. This process of re-screening, mutation, and re-delivery enables directed evolution selection for potently neutralizing antibodies.

### Example 12: Antibody Variant Neutralization of Many Viral Strains Sequentially

In this prophetic example, we use SARS-CoV-2 Wuhan Hu-1 strain as our pseudovirus for neutralization analysis to isolate the neutralizing antibodies from method described in Examples 10 and 11. We can recover the neutralizing IgG libraries expressing HEKACE2 cells through recovering the GFP-cells. We use another virus mutation variant such as S-D614G variant to perform sequential neutralization screening (Defined as second round) via cell isolation platforms as described in Example 7, Example 8 or Example 9. After the second round of screening, the populations are enriched for antibodies that exhibit neutralizing capabilities against both Wuhan Hu-1 and D614G.

Alternatively, after the FACS post sorting of GFP negative cells we extract the RNA from these cells and perform RT-PCR to obtain the VH:VL sequences. We then re-deliver the VH:VL pair into the HEKACE2 cells described at Example 3 to generate the secreted protein library after a single library sort. We then perform the second round of screening using the pseudovirus variant contain D614 mutation. These methods allow for the selection of neutralizing antibodies targeting multiple viral strains of interest.

### Example 13: Antibody Variant Neutralization with Many Viral Strains Concurrently

In this prophetic example, perform the pseudovirus neutralization using multiple virus strains at the same time. We first mix an equal amount of the virus from broad coronavirus strains, including SARS-CoV-2, SARS-Cov-2-D614G, SARS-CoV-1, MERS-CoV, with each pseudovirus contain YFP, GFP, DsRed and CFP, respectively. Alternatively, we can use different viral strains all derived from different SARS-CoV-2 variants (e.g., B.1.1.7, B.1.351, P.1, B.1.427, and B.1.429). In some embodiments, all viruses encode for the same reporter (e.g., GFP). In some embodiments, each virus encodes for a different DNA or RNA barcode that the target cells will express after infection. In some embodiments, authentic virus is used. In other embodiments, pseudovirus is used. We perform neutralization assays of antibody libraries with mixture of viral strains based on approaches described in Example 7 (multiple well plates based), Example 8 (microchamber based) or Example 9 (microfluidic droplet based). For multiple-well plate assay in Example 7, we choose the well-containing cells showed no YFP, GFP, DsRed and CFP as candidate cells that express antibodies with broad neutralization. Other fluorescent markers can be used and are known to those skilled in the art. In both microchamber (Example 8) and microfluidic droplet-based methods (Example 9), after we retrieve cells from either microchambers (Example 8) or droplets (Example 9), we rest and expand cells for another 48 hours (the cells can be rested for any amount of time between 0 hours and multiple months depending on the experimental preference). We sort the cells with no YFP, GFP, DsRed and CFP expression, and also the cells that show fluorophore expression (i.e., were infected). We obtain the VH:VL pairing information of each population through RT-PCR gene recovery and high-throughput sequencing. We compare the sequences of both screening populations, and we then express the candidate antibodies enriched in the populations no YFP, GFP, DsRed and CFP from the HEK293Expi cells and purify the antibody for quantification. We evaluate individual antibody's neutralization capability against SARS-CoV-2, SARS-Cov-2-D614G, SARS-CoV-1, and MERS-CoV according to the method described in Example 10.

### Example 14: Antibody Variant Neutralization with Many Different Viruses Concurrently

In this prophetic example, we perform the pseudovirus neutralization using multiple different virus types at the same time. We first mix an equal amount of the virus from different strains, including SARS-CoV-2, SARS-Cov-2-D614G, YFV, and DENV-1, with each pseudovirus contain YFP, GFP, DsRed and CFP, respectively. In some embodiments, all viruses encode for the same reporter (e.g., GFP). In some embodiments, each virus encodes a different reporter. In some embodiments, each virus encodes for a different DNA or RNA barcode that the target cells will express after infection. In some embodiments, authentic virus is used. In other embodiments, pseudovirus is used. A cell line is generated that can be infected by any of the viruses used. In some embodiments, a cell that can be infected with SARS-CoV-2, SARS-Cov-2-D614G, YFV, and DENV-1 is generated by starting with Raji-DC-SIGN cells, which are used for in vitro infections with YFV and DENV-1 recombinant viral particles (RVPs) and modify Raji-DC-SIGN to express the ACE2 protein that enables infection also with SARS-CoV-2. We next clone a library of antibodies to express and secrete antibody from the modified Raji-DC-SIGN-ACE2 cells and perform neutralization assays of antibody libraries with mixture of viruses based on approaches described in Example 7 (multiple well plates based), Example 8 (microchamber based) or Example 9 (microfluidic droplet based).

For multiple-well plate assay in Example 7, we choose the well-containing cells showed no YFP, GFP, DsRed and CFP as candidate cells that express antibodies with broad neutralization. In both microchamber (Example 8) and microfluidic droplet-based methods (Example 9), after we retrieve cells from either microchambers (Example 8) or droplets (Example 9), we rest and expand cells for another 48 hours (although cells can be rested from anywhere in between 0 hours and multiple months, depending on the preferences of the experiment). We sort the cells with no YFP, GFP, DsRed and CFP expression, and also the cells that show fluorophore expression (i.e., were infected). We obtain the VH:VL pairing information of each population through RT-PCR gene recovery and high-throughput sequencing. We compare the sequences of both screening populations, and we then express the candidate antibodies enriched in the populations no YFP, GFP, DsRed and CFP from the HEK293Expi cells to determine the sequences of neutralizing antibodies using a high-throughput assay. In some embodiments, each virus encodes a cell-specific barcode that encodes for the virus type, allowing for a high-throughput DNA-based readout of the infecting viruses in the library of bulk or single cells, in addition to high-throughput analysis of the antibody gene sequences in the infected or non-infected antibody populations. In some embodiments, single cell sequencing is used to link the barcode of the infecting virus to the DNA sequence of the antibody directly.

### Example 15: Rapid High-Throughput Discovery of Secreted Proteins Activating 4-1BB

In this prophetic example, a cell line is used for 4-1BB expression along with a reporter that causes expression of a fluorescent marker or other reporter (for example, GFP or other cellular selection markers known in the art) when 4-1BB is activated. A fusion protein of 4-1BB extracellular domain is generated with an internal activation signal that causes GFP expression when 4-1BB is activated. A protein library is encoded in the cell line (one protein variant per cell) that causes each cell to secrete the protein variant. The cells are isolated as single cells inside compartments and allowed to incubate for 4 hours to accumulate secreted protein (although the time can range from seconds to months, depending on the conditions and goals of the experiment). In some embodiments, the compartments are comprised of emulsion droplets. The cells that secrete protein that activate 4-1BB will activate fluorescent marker expression (for example, GFP). After cell recovery, the marker+ and marker− cells are isolated via flow cytometry, and their identities characterized by DNA sequencing to determine the protein variants within the library that can functionally activate 4-1BB. As an alternative approach, a luciferase detection system could be used in place of fluorescent cell sorting to detect secreted proteins with functional activities of interest. After the identification of appropriate secreted proteins with functional activities of interest, the discovered proteins would have a potential as immunotherapies to activate 4-1BB for the treatment of cancer or other diseases.

### Example 16: Rapid High-Throughput Discovery of Secreted Proteins Blocking Programmed Death Receptor 1 (PD-1) Activation

In this prophetic example, a cell line is generated for PD-1 expression along with a selectable marker that causes expression of a fluorescent reporter or other reporter (for example, GFP or other cellular selection markers known in the art) when PD-1 is activated. A fusion protein of PD-1 extracellular domain is generated with an internal activation signal that causes GFP expression when PD-1 is activated. A protein library is encoded in the cell line (one protein variant per cell) that causes each cell to secrete the protein variant. The cells are isolated as single cells inside compartments and allowed to incubate for 4 hours to accumulate secreted protein (although the time can range from seconds to months, depending on the conditions and goals of the experiment). In some embodiments, the compartments are comprised of emulsion droplets. Then, PD-L1 is added to the compartments to induce the ligation and activation of PD-1. The cells that secrete protein that blocks PD-L1 binding and/or prevents PD-1 activation will prevent the fluorescent marker from being expressed. After cell recovery, the GFP-cells are isolated via flow cytometry, and their identities characterized by DNA sequencing to determine the protein variants within the library that can block PD-1 activation via PD-L1. As an alternative approach, a luciferase detection system could be used in place of fluorescent cell sorting to detect secreted proteins with functional activities of interest. After the identification of appropriate secreted proteins with functional activities of interest, the discovered proteins would have a potential ability to be immunotherapeutic checkpoint inhibitors for cancer treatment.

### Example 17: Rapid High-Throughput Discovery of Secreted Proteins Blocking GPCR Activation

In this prophetic example, a cell line is generated for G protein coupled receptor (GPCR) expression along with a reporter that causes expression of a fluorescent marker (or other reporter (for example, GFP or other cellular selection markers known in the art) when the GPCR is activated. A GPCR is expressed in a cell line that activates an internal activation signal when the GPCR is activated. Example cell lines are commercially available (e.g., GPCR cell lines from the Eurofins company) or can be similarly built. A secreted protein library is also encoded in the cell line (one protein variant per cell) that causes each cell to secrete the protein variant. The cells are isolated as single cells inside compartments and allowed to incubate for 4 hours to accumulate secreted protein (although the time can range from seconds to months, depending on the conditions and goals of the experiment). In some embodiments, the compartments are comprised of emulsion droplets. Then, a GPCR agonist is added to the compartments to induce the ligation and activation of the GPCR. The cells that secrete protein that blocks GPCR agonist binding and/or prevents GPCR activation will prevent the fluorescent marker from being expressed. After cell recovery, activated- and non-activated cells are isolated via flow cytometry, and their identities characterized by DNA sequencing to determine the protein variants within the library that can block GPCR activation. As an alternative approach, a luciferase detection system could be used in place of fluorescent cell sorting to detect secreted proteins with functional activities of interest. After the identification of appropriate secreted proteins with functional activities of interest, the discovered proteins would be promising candidates as drugs to block GPCR activation.

### Example 18: Rapid High-Throughput Discovery of Secreted Proteins Inducing GPCR Activation

In this prophetic example, a cell line is generated for G protein coupled receptor (GPCR) expression along with a reporter that causes expression of a fluorescent reporter or other reporter (for example, GFP or other cellular reporters known in the art) when the GPCR is activated. A GPCR is expressed in a cell line that generates an internal activation signal that when the GPCR is activated. Example cell lines are commercially available (e.g., GPCR cell lines from the Eurofins company) or can be similarly built. A secreted protein library is encoded in the cell line (one protein variant per cell) that causes each cell to secrete the protein variant. The cells are isolated as single cells inside compartments and allowed to incubate for 4 hours to accumulate secreted protein (although the time can range from seconds to months, depending on the conditions and goals of the experiment). The cells that secrete protein that activate GPCR will cause the fluorescent marker to be expressed in those same cells. After cell recovery, activated- and non-activated cells are isolated via flow cytometry, and their identities characterized by DNA sequencing to determine the protein variants within the library that activate GPCRs. As an alternative approach, a luciferase detection system could be used in place of fluorescent cell sorting to detect secreted proteins with functional activities of interest. After the identification of appropriate secreted proteins with functional activities of interest, the discovered proteins would be promising candidates as drugs to activate GPCRs.

### Example 19: Secreted Protein Analysis for Neutralization of Yellow Fever Virus

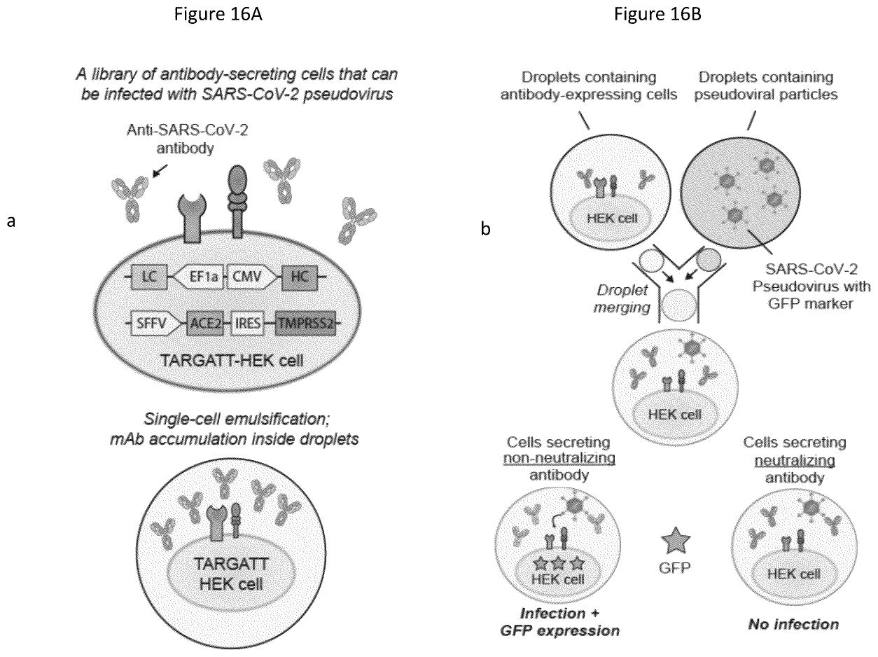

In this working example, Raji-DCSIGNR cells were used to test the ability of secreted proteins to neutralize yellow fever virus (YFV). In this example we expressed antibody in the bi-cistronic format using a p2a motif as described in Example 5, using lentiviral transduction to insert genes into the cells for secretion. We utilized lentiviral transduction of Raji-DCSIGNR cells to evaluate their capacity for high-throughput single-cell neutralization assays. We transduced Raji-DCSIGNR cells⁽¹⁶⁾ with a yellow fever virus neutralizing antibody, mAb-17, for antibody secretion, or with an empty plasmid that would not induce antibody expression. We tested the cell lines after 4 days of antibody secretion in 96-well plates before adding YFV recombinant viral particles (RVP) to verify that the secreted antibody would provide protection from YFV RVP (FIG. 17). The cells expressing mAb 17 were protected from YFV RVP infection, whereas cells that were not expressing mAb 17 were unprotected from infection. These data confirmed that we can link antibody secreted protein functional neutralization properties to a GFP-based reporter (that is expressed after the RVP infection event) as a cell line platform for direct screening of anti-YFV antibody neutralization in a rapid, high-throughput manner.

### Example 20: Antibody Expression with Different Leader Peptide and Promoter Combinations

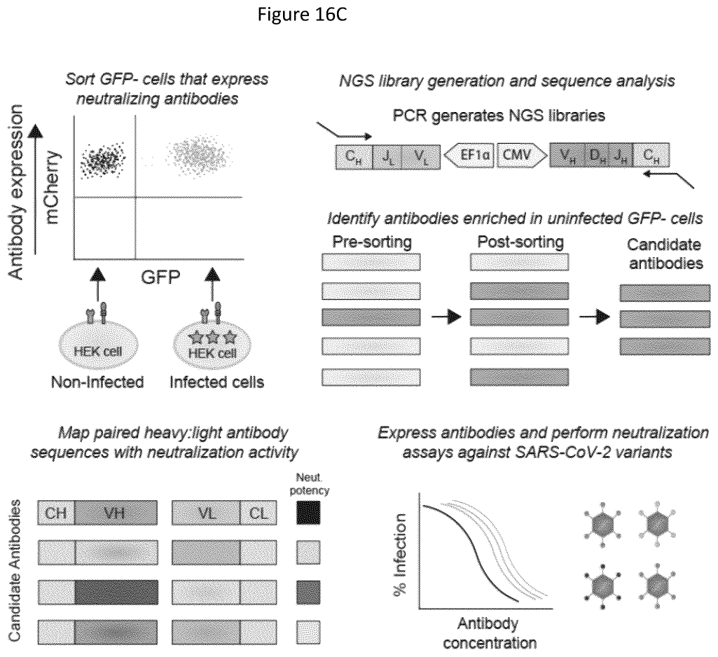

In this working example we tested the ability of HEK293 cells expressing ACE2 to be transiently transfected with plasmids containing 910-30 expressed with different leader peptide combinations (LP1, LP4, LP5 and LP6) for antibody secretion. Lipofectamine 3000 was used as a transfection reagent following the reverse transfection protocols in 96 well plates and incubated for two days at 37° C. Two days post-transfection, 40 μL of SARS-CoV-2 pseudovirus with GFP reporter gene was added to the cells and incubated at 37° C. for another three days. Three days after adding the pseudovirus, the supernatant containing secreted IgG is removed from the cells for use in ELISA antibody quantification. The ELISA readout is illustrated in FIG. 18. Antibodies VRC01 and CR3022 expressed by numerous peptide combinations showed successful antibody expression, indicating the successful secretion of antibodies using different leader peptides and promoter sequences.

### Example 21: Use of CRISPR-Cas9 to Clone Antibodies into Soluble Protein Cell Secretion Platforms

In this working example, we cloned an anti-SARS-Cov2 monoclonal antibody, 2-15, into a donor vector AAVS1 Safe Harbor Targeting Knock-in HR Donor 2 vector, GE622A-1, from System Biosciences. We named the donor plasmid with 2-15 monoclonal antibody, pGE622A2-15. We then co-transfected the 2-15 donor plasmid (pGE622A2-15) and the All-in-one Cas9 Smart Nuclease AAVS1 Targeting Plasmid (System Bioscience #CAS601A-1) into the Expi293 cells. The expression of the Cas9 nuclease and the gRNA after the transfection generated a double strain break at the Expi293 cell AAVS1 genome site. 2-15 gene sequence from the donor plasmid was integrated into the AAVS1 gene locus because of homologous recombination event (See illustration of the 2-15 gene integration below). We began the puromycin selection (at a concentration of 5 μg/mL) 1-week post-transfection to reduce the random integration Expi293 cells. The stable cell pool with 2-15 gene integration was named Expi2-15.

We seeded the Expi2-15 and Expi293 cells into a 96-well plate with a density of 3.2×10⁴ cells per well. We then transfected the cell with ACE2/TMRPSS2 expressing plasmid right after seeding these cells (both Expi2-15 and Expi293). For the positive control of the neutralization assay, we added purified 91030 antibody (5 μg/mL of final concentration) into ACE2/TMRPSS2 expressing Expi293 cells. We aliquoted 20 μL of the culture media from each well for further IgG quantification analysis. We added 80 μL of the SARS CoV-2 reporter virus particle with spike protein D614G mutation and luciferases reporter gene to the ACE2/TMPRSS2 expressing Expi2-15 cells (ACE2/TMPRSS2+Expi2-15), ACE2/TMPRSS2 expressing wild-type Expi293 cells (ACE/TMPRSS2+Expi293) and ACE/TMPRSS2 expressing wild-type Expi293 cells with 5 μg/mL of 91030 (ACE/TMPRSS2+Expi293+91030).

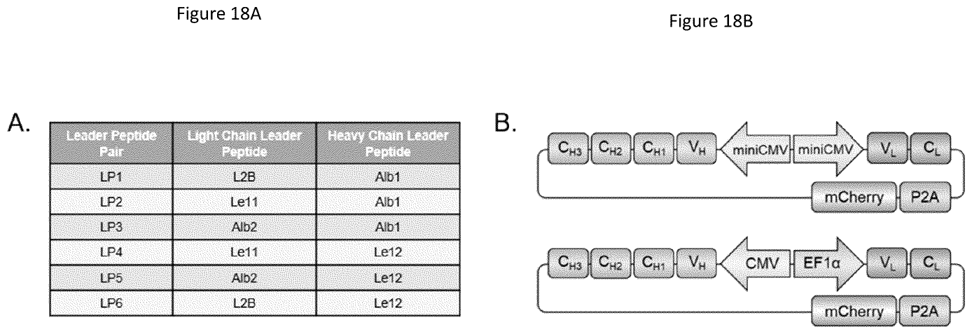

Three days post adding the reporter virus, we removed the culture media and added 30 μL of PBS and 30 μL of diluted Renilla-Glo Assay Substrate (diluted Renilla-Glo Assay Substrate into the Assay Buffer at 1:100). We then detected luminescence in a luminometer after 10 minutes of incubation at room temperature. We calculated the average relative light unit (RLU) of the luminometer reading of each group. As shown in the figure below, both ACE2/TMPRSS2+Expi2-15 and ACE/TMPRSS2+Expi293+91030 (Positive Control) groups showed a significant reduction in relative light units as compared with that of the ACE/TMPRSS2+Expi293 group, indicating that 2-15 secreted from Expi2-15 cells is able to neutralize the SARS-CoV2 pseudovirus (FIG. 20).

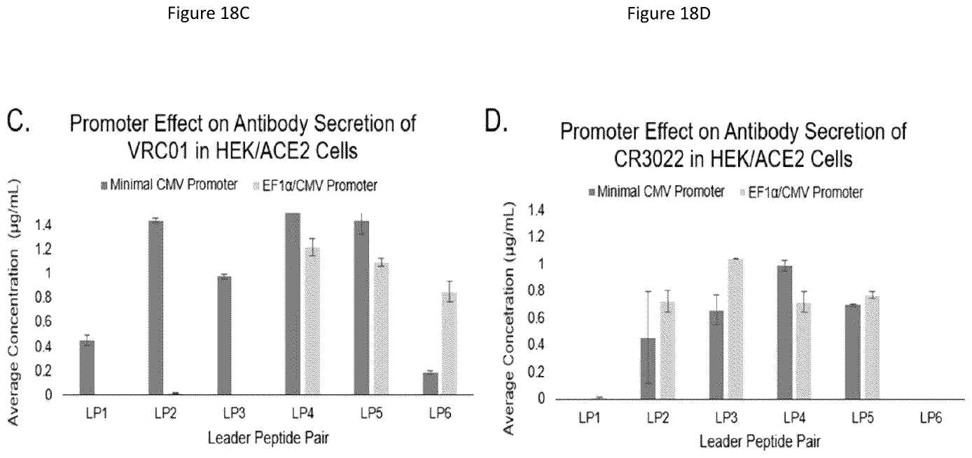

We validated the antibody expression level of the ACE2/TMPRSS2+Expi2-15 group via ELISA. The average antibody expression level is 0.23 μg/mL (n=6) antibody expression from the ACE/TMPRSS2+Expi2-15, suggesting that Expi2-15 cells are able to secret functionally active 2-15 (FIG. 21). The group, ACE/TMPRSS2+Expi293+91030, containing 5 μg/mL of the purified 91030 monoclonal antibody (calculated based on nanodrop of the purified 91030) was measured at a concentration of 3.9 μg/mL via ELISA as internal control for our ELISA assay.

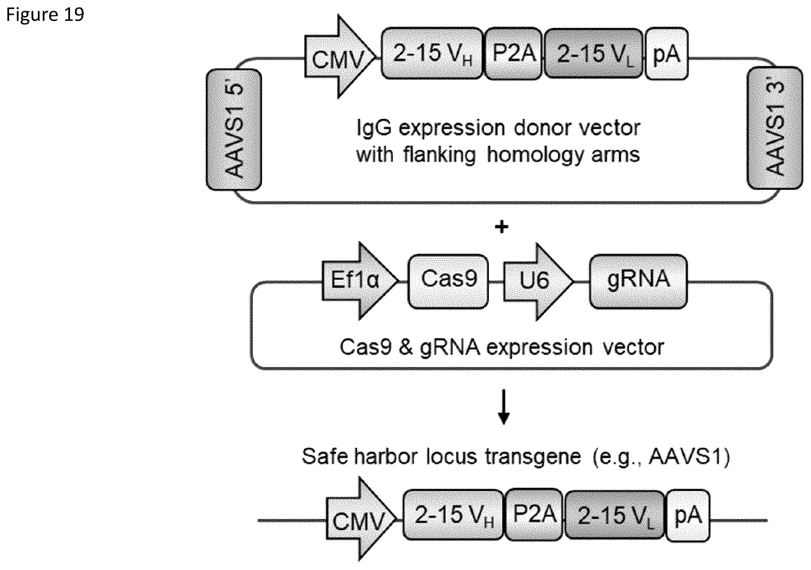

We further performed genomic PCR to validate the integration of the 2-15 mab gene sequencing into the Expi2-15 cell line. We first isolated the genomic DNA from wild-type Expi293, and Expi2-15 cell lines. Then we performed PCR amplification to amplify the upstream gene integration region using GoTaq2 hot-start polymerase (Promega #M7405) and primers (Upstream primer set, Forward 5′ TCCTGAGTCCGGACCACTTT 3′ (SEQ ID NO: 25) and Reverse 5′ CACCGCATGTTAGAAGACTTCC 3′ (SEQ ID NO: 26)) validated and provided by the System Bioscience. A 1000 b.p. amplicon from the Expi2-15 cells indicated a successful gene integration compared with no PCR amplification from the wild-type Expi293 cells (see FIG. 22a).

A separate PCR reaction using a human control primer set (Forward 5′-ACCTCCAGTTAGGAAAGGGGACT-3′ (SEQ ID NO: 27) Reverse 5′-AAGTTTTTCTTGAAAACCCATGGAA-3′ (SEQ ID NO: 28)) for internal PCR control (FIG. 22b).

### Example 22: Use of TARGATT Specific Integration to Clone Antibodies into Soluble Protein Cell Secretion Platforms

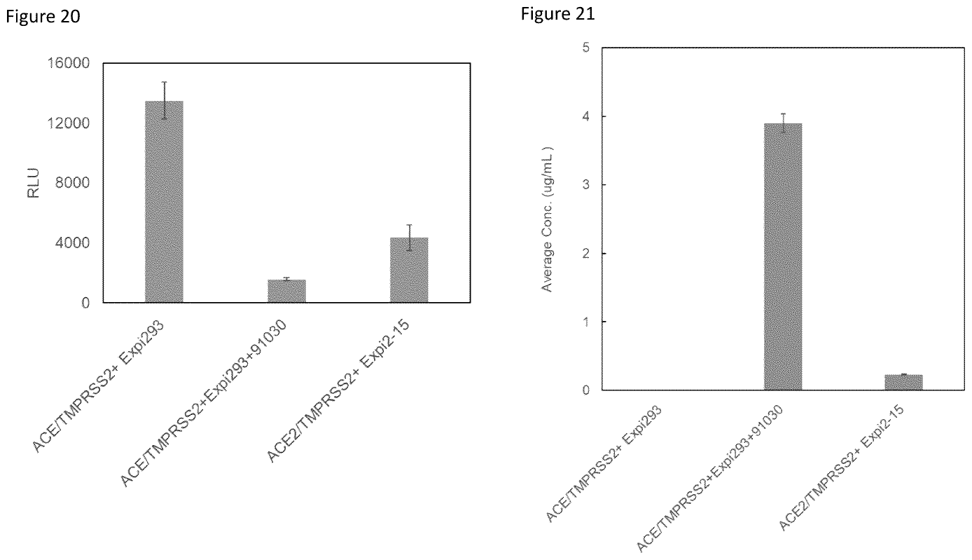

In this working example, we followed the instruction manual of the TARGATT™ HEK master cell line knock-in kit to clone an anti-SARS-Cov2 monoclonal antibody, 2-15, into the TARGATT 24 CMV-MCS-attB (named pTARGATT2-15) to produce the donor plasmid. We then co-transfected the 2-15 donor plasmid (pTARGATT2-15) and the integrase plasmid into TARGATT HEK master cells. The integrase catalyzes a gene recombination event allowing the integration of 2-15 monoclonal antibody, mCherry and blasticidin selectable marker into genome (See FIG. 23).

Three days after transfection, we sub-cultured the transfected cell with a split ratio of 1:20. Twenty-four hours after the sub-culture, we added blasticidin in a concentration of 10 μg/mL and maintained blasticidin selection pressure for two weeks. We then performed cell sorting to isolate the mCherry positive cells to enrich the 2-15 integrated cells (named TARGATT2-15). After the recovery of the TARGATT2-15 cells, we seeded the TARGATT2-15 and wild-type TARGATT cells into a 96-well plate with a density of 3.2×10⁴ cells per well. We then transfected the cell with ACE2/TMRPSS2 expressing plasmid right after seeding the cells (as described in Example 1 and in FIG. 2). Two days after the transfection, we added purified 91030 antibody (5 μg/mL of final concentration) into unmodified TARGATT cells as positive control prior to the pseudovirus neutralization assay. We aliquoted 20 μL of the culture media from each well for further IgG quantification analysis.

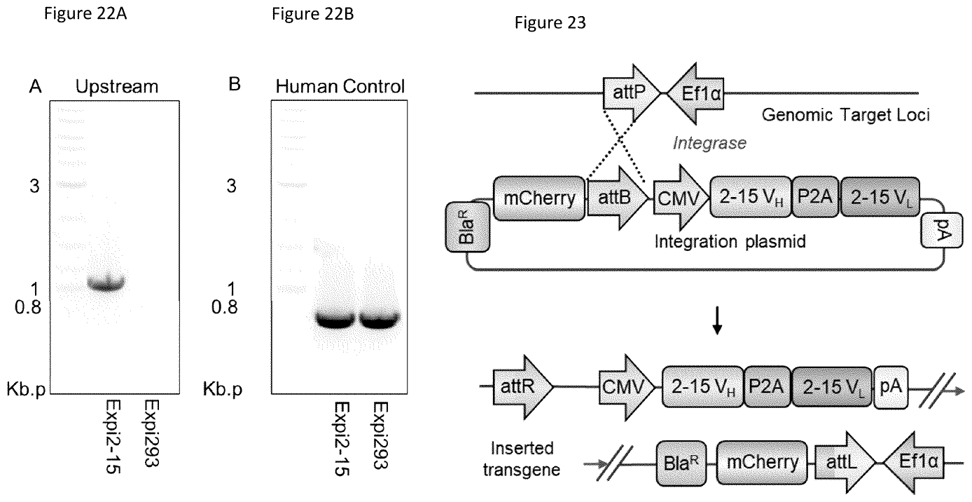

We added 80 μL of the SARS CoV-2 reporter virus particle with spike protein D614G mutation and luciferases reporter gene to the ACE2/TMPRSS2 expressing TARGATT2-15 cells (ACE2/TMPRSS2+TARGATT2-15), ACE2/TMPRSS2 expressing wild-type TARGATT cells (ACE/TMPRSS2+TARGATTWT) and ACE/TMPRSS2 expressing wild-type TARGATT cells with 5 μg/mL of purified 91030 (ACE/TMPRSS2+91030). Three days post adding the reporter virus, we removed the culture media and added 30 μL of PBS and 30 μL of diluted Renilla-Glo Assay Substrate (diluted Renilla-Glo Assay Substrate into the Assay Buffer at 1:100). We then detected luminescence in a luminometer after 10 minutes of incubation at room temperature. We calculated the average relative light unit (RLU) of the luminometer reading of each group. As shown in the figure below, both ACE2/TMPRSS2+2-15 and ACE/TMPRSS2+91030 groups showed a significant reduced in relative light unit as compared with that of the ACE/TMPRSS2+WT group, indicating that 2-15 secreted from TARGATTHEK2-15 cells is able to neutralize the SARS-CoV2 pseudovirus (FIG. 24).

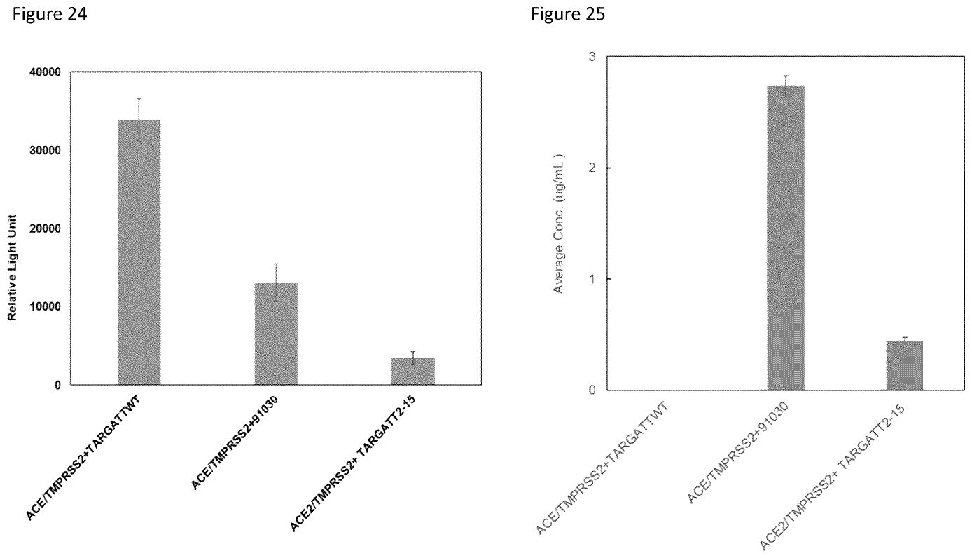

We validated the antibody expression level of the ACE2/TMPRSS2+TARGATT2-15 group via ELISA. The average antibody expression level is 0.44 μg/mL (n=6) antibody expression from the TARGATT2-15, demonstrating that TARGATT2-15 cells are able to secret functionally active 2-15 (see FIG. 25). The group, ACE/TMPRSS2+91030, containing 5 μg/mL of the purified 91030 monoclonal antibody (calculated based on nanodrop of the purified 91030) was measured at a concentration of 2.74 μg/mL via ELISA as internal control for our ELISA assay.

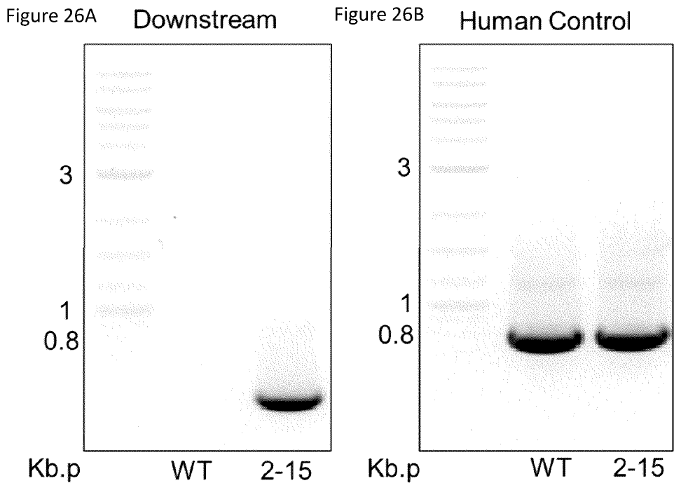

We further performed genomic PCR to validate the integration of the 2-15 mab gene sequencing into the TARGATT2-15 cell line. We first isolated genomic DNA from wild-type TARGATT and TARGATT2-15 cell lines. Then we performed PCR amplification to amplify the downstream gene integration region using GoTaq2 hot-start polymerase (Promega #M7405) and primer sequences (Downstream primer set, Forward 5′ CCTTGTAGATGAACTCGCCGT 3′ (SEQ ID NO: 29) and Reverse 5′ GGTGTCGTGATTATTCGAAGGG 3′ (SEQ ID NO: 30)) validated and provided by the Applied StemCell, Inc. A 500 b.p. amplicon from the TARGATT2-15 group indicated a successful gene integration compared with no PCR amplification from the wild-type TARGATT cells (FIG. 26).

### Example 23: Use of Rapid Droplet-Based Assays to Identify Neutralizing Antibodies Using Next-Generation Sequencing

In this prophetic example, we clone antibodies into Raji-DCSIGNR cells and generate a synthetic library mixture to test the ability of droplet-based screening to identify neutralizing antibodies targeting yellow fever virus (YFV). We utilize lentiviral transduction of Raji-DCSIGNR cells to evaluate their capacity to be used in high-throughput single-cell neutralization assays. We transduce Raji-DCSIGNR cells with a yellow fever virus neutralizing antibody, mAb-17, for antibody secretion, or with other antibodies (910-30, VRCO1, and 2-15) that do not neutralize YFV. We encapsulate the cells in microfluidic droplets and incubate them for 24 hours (although the incubation time could range from minutes to several weeks depending on the goals of the experiment) to facilitate antibody secretion and accumulation within the droplet. Next, we merge the droplets using the electrocoalescence technique (other techniques for droplet merging can also be used and are known to those skilled in the art) and incubate overnight at 37 degrees Celsius to allow the pseudovirus to infect any cells that are not protected by secreted antibodies (the amount of incubation time and the temperature of incubation can vary according to the goals of the experiment). Droplets are broken, and the cells are recovered. After a brief incubation time (which can range from 0 minutes to several weeks depending on the goals of the experiment), we sort GFP+ and GFP-cells on a flow cytometer to separate the neutralizing and non-neutralizing cells. Cells are collected and genomic DNA is extracted for PCR-based amplification.

DNA is sent for next-generation sequencing to quantify the prevalence of each antibody clone in the dataset. The neutralizing antibodies are enriched in the set of GFP-cells, and depleted in the GFP+ cells, and neutralizing antibodies could be identified based on these enrichment features. These data will confirm that we can link antibody secreted protein functional neutralization properties to a reporter (that is expressed after the recombinant viral particle, RVP, infection event) as a cell line platform for direct screening of anti-YFV antibody neutralization in a rapid, high-throughput manner, and furthermore, that the sequences of neutralizing antibodies can be detected using next-generation sequencing analysis.

### Example 24: Secreted Protein Analysis for Neutralization of HIV-1

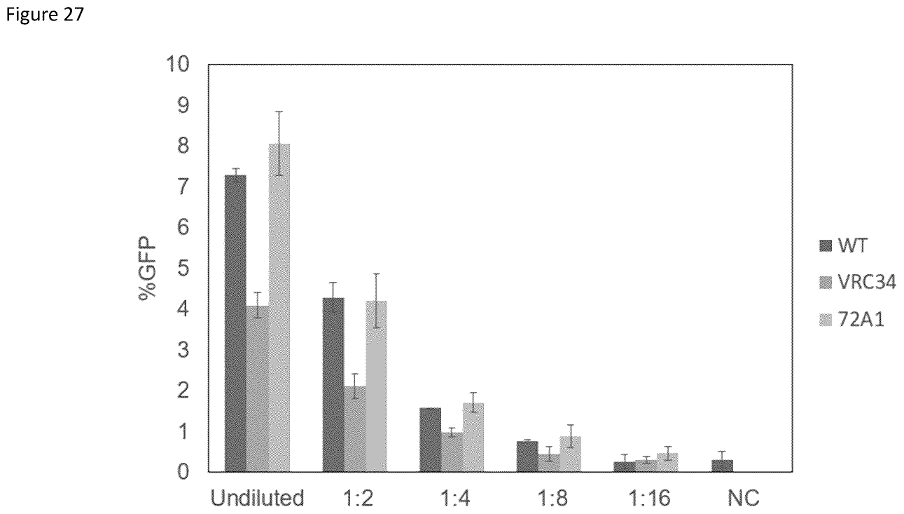

In this working example, TZM-GFP cells were used to test the ability of secreted proteins to neutralize human immunodeficiency virus 1 (HIV-1). We utilized lentiviral transduction TZM-GFP cells to evaluate their capacity for high-throughput single-cell neutralization assays. We transduced TZM-GFP cells⁽²³⁾ with an HIV-1 neutralizing antibody, VRC34, for antibody secretion, or with a control antibody that does not neutralize HIV-1 (72A1) We tested the cell lines after 2 days of antibody secretion in 96-well plates before adding HIV-1 pseudovirus particles (strain W6M.EnV.C2) to verify that the secreted antibody would provide protection from HIV-1 pseudoviruses (FIG. 27). The cells expressing VRC34 were protected from HIV-1 pseudovirus infection, whereas cells that were not expressing VRC34 were unprotected from infection. These data confirmed that we can link antibody secreted protein functional neutralization properties to a GFP-based reporter (that is expressed after the pseudovirus infection event) as a cell line platform for direct screening of anti-HIV-1 antibody neutralization in a rapid, high-throughput manner.

### Example 25: Droplet Merging Techniques to Enable Soluble Secretion Assays Inside Droplets with a Secreted Protein Cell Library

In this working example, we applied droplet merging techniques to demonstrate the encapsulation and droplet merger, and the recovery of DNA from cell libraries, to enable secretion cell assays. We first generated a synthetic cell library, where each cell secretes a separate antibody clone and also expresses ACE2, that could be used to screen for secreted protein function. We mixed four different cell groups expressing antibody clones into a single library (Table 3).

TABLE 3
Cells expressing known antibody clones were mixed and used
as artificial cell libraries. HEK293-T clones expressing ACE2,
and different monoclonal antibody clones were mixed as shown at
1 × 10⁶ cells/mL in High glucose DMEM supplemented
with 5% fetal bovine serum and 1% penicillin-streptomycin.

| Clone | Cell number | % of library |
|---|---|---|
| HEK/ACE2 VRC01 | 3,960,000 | 99% |
| HEK/ACE2 CR3022 | 13,333 | 0.33% |
| HEK/ACE2 910-30 | 13,333 | 0.33% |
| HEK/ACE2 1-20 | 13,333 | 0.33% |
| Total | 4,000,000 | 100% |

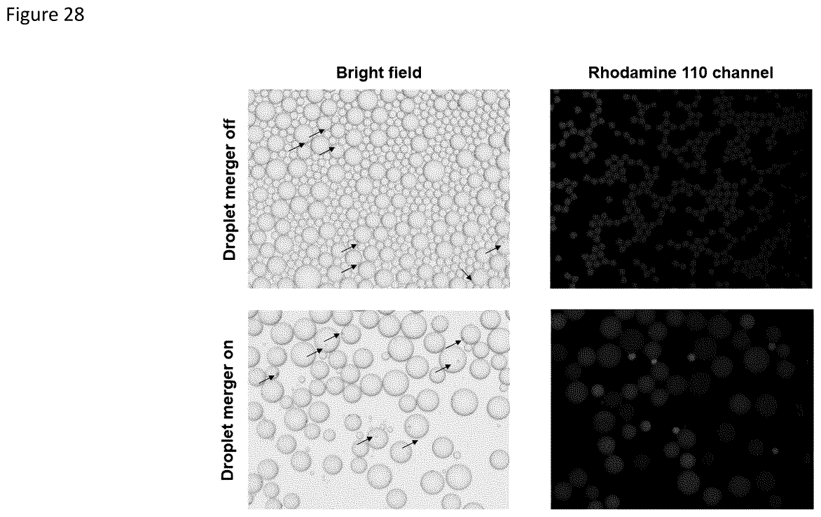

Cells were captured into single cell emulsions using a droplet generator (F02-HPB-8x, uFluidix, Canada), which generates −80 μm diameter droplets. Next, droplets were loaded into a droplet merging device that applies an electric field to induce the merging of droplets. This device also generates droplets containing rhodamine 110 (diameter: −40 μm. #83695, Sigma-Aldrich, USA) for merging with the cell droplets. (FIG. 28).

### Example 26: Recovery of DNA to Identify Secreted Proteins in Cell Populations Sorted with Different Selection Markers after Soluble Protein Secretion Assays

In this working example, we applied droplet merging techniques to demonstrate the encapsulation and droplet merger, and the recovery of DNA from cell libraries, to enable secretion cell assays. We generated and sorted a synthetic cell library (Table 3), with four different antibody clones, only some of which can potently neutralize SARS-CoV-2.

After droplet merger with SARS-CoV-2 pseudovirus that induces GFP expression in infected cells, cells were recovered from emulsions and sorted for expression of the GFP marker that indicates functional performance differences among the secreted antibodies in the library. In this case, the functional screen identified neutralizing antibodies, comparatively enriched in the GFP-cell population, and non-neutralizing antibodies were contained in the GFP+ cell population.

Genomic DNA was isolated from HEK cells using Quick-DNA Miniprep Kit (Zymo Research, USA). Next, heavy chain variable regions were amplified using Platinum Taq DNA Polymerase (ThermoFisher Scientific, USA) using primers anchoring the 3′ region of the cytomegalovirus promoter and the 5′ region of the heavy constant chain. The primer sequences used were: Forward: 5′-GGTGGGAGGTCTATATAAGCA-3′ (SEQ ID NO: 31), Reverse: 5′-CCAGAGGTGCTCTTGGAG-3′ (SEQ ID NO: 32). Polymerase chain reaction was carried out during 40 cycles using 51° C. as annealing temperature. PCR products were resolved in a 1% agarose gel, using a 1 Kb DNA ladder (#N0550S, New England BioLabs, USA) to control for size. The resulting DNA gels are shown in FIG. 29. These data demonstrate our ability to recover the DNA sequences from cells utilized in high-throughput droplet-based cell secretion protein functional assays.

### Example 27 Application of the Single-Cell Assay Using a Synthetic Library of Antibodies with Known Neutralization Properties Against SARS-CoV-2 Inside Emulsion Droplet Systems

This working Example relates to the successful screening of a synthetic cell library secreting antibody molecules for the neutralization of SARS-CoV-2 pseudovirus. First, we mixed HEK-ACE2 expressing different monoclonal antibodies to generate a synthetic library consisting of 4 antibody-producing cells (the previously reported antibodies VRCO1, CR3022, 910-30 and mAb1-20); VRC01 does not neutralize SARS-CoV-2 and serves as a negative control. We used a microfluidic device to encapsulate the synthetic library with DMEM media to form droplets, with one cell per droplet. We incubated the droplet for 24 hours, allowing the secretion of IgG within the droplet for antibody accumulation. Subsequently, a second droplet containing D614G SARS-CoV-2 pseudovirus was merged into the droplet with IgG expressing HEK-ACE2 cells. We used electrocoalescence to merge droplets, although alternative methods to merge droplets have been reported including the use of micropillar resistance arrays. The merged droplets were further incubated for another 24 hours to allow pseudovirus infection or neutralization to occur. The droplets were then broken, and the cells are recovered. Cells were allowed to recover for 48 hours. We isolated GFP- and GFP+ populations using fluorescence activated cell sorting (FACS) and extracted the gDNA from cell aliquots and performing PCR to recover the antibody gene libraries for NGS analysis. GFP-cells were also recovered and used as input for a subsequent round of screening for further enrichment for neutralizing clones.

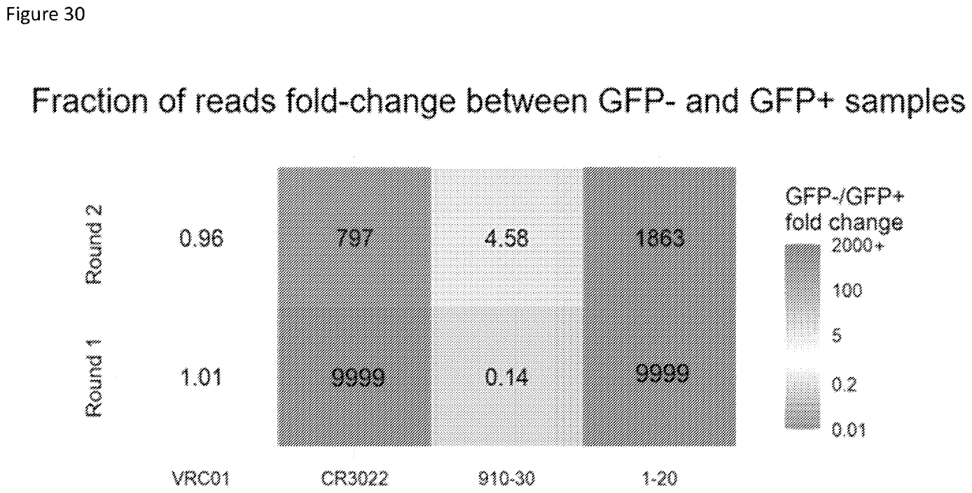

We performed high-throughput sequencing analysis on each sorted library of GFP- and GFP+ cells to obtain heavy chain sequence information. We compared the frequency of heavy chain antibody variants in each population to determine the effect of the droplet neutralization assay on neutralizing and non-neutralizing antibodies in the population (FIG. 30, Table 4).

TABLE 4
Raw sequence data and fold-change enrichment calculations for SARS-CoV-2 D614G
neutralization assays. These data demonstrate the successful implementation of
droplet neutralization assays for antibodies that neutralize SARS-CoV-2,
with NGS being used to analyze the assay performance for many thousands
of cells secreting polypeptide molecules in parallel.
SARS-CoV2 Sort Round 1

| | | | | Fraction of | | | Fold |
|---|---|---|---|---|---|---|---|
| # reads | | | | total reads | | | change |
| Round 1 | Presort | GFP− | GFP+ | Presort | GFP− | GFP+ | GFP−/GFP+ |
| VRC0l | 453,933 | 796,018 | 152,018 | 0.99474 | 0.9965 | 0.99153 | 1.005 |
| CR3022 | 204 | 106 | — | 0.00045 | 0.00013 | 0 | #DIV/0! |
| “910-30” | 1,746 | 946 | 1,298 | 0.00383 | 0.00118 | 0.00847 | 0.140 |
| “1-20” | 452 | 1,742 | — | 0.00099 | 0.00218 | 0 | #DIV/0! |
| Total | 456,335 | 798,812 | 153,316 | 1 | 1 | 1 | |

SARS-CoV2 Sort Round 2

| | | | | Fraction of | | | Fold |
|---|---|---|---|---|---|---|---|
| # reads | | | | total reads | | | change |
| Round 2 | Presort | GFP− | GFP+ | Presort | GFP− | GFP+ | GFP−/GFP+ |
| VRC0l | 1,843,790 | 1,345,524 | 985,847 | 0.97891 | 0.95775 | 0.99399 | 0.964 |
| CR3022 | 3,759 | 2,259 | 2 | 0.002 | 0.00161 | 2E−06 | 797.399 |
| “910-30” | 20,351 | 38,624 | 5,955 | 0.0108 | 0.02749 | 0.006 | 4.579 |
| “1-20” | 15,604 | 18,474 | 7 | 0.00828 | 0.01315 | 7.lE−06 | 1863.169 |
| Total | 1,883,504 | 1,404,881 | 991,811 | 1 | 1 | 1 | |

### Example 28: Application of the Single-Cell Assay Using a Synthetic Library of Antibodies with Known Neutralization Properties Against HIV Pseudoviruses Inside Emulsion Droplet Systems

This working Example relates to the successful screening of a synthetic cell library secreting antibody molecules for the neutralization of HIV pseudovirus. First, we mixed TZM-GFP cells expressing different monoclonal antibodies to generate a synthetic library consisting of 3 antibody-producing cells (the previously reported antibodies 72A1, VRCO1, and VRC34); 72A1 does not neutralize HIV-1 and serves as a negative control. We used a microfluidic device to encapsulate the synthetic library with media to form droplets, with one cell per droplet. We incubated the droplet for 24 hours, allowing the secretion of IgG within the droplet for antibody accumulation. Subsequently, a second droplet containing HIV-1 BG505.W6M.Env.C2 pseudovirus was merged into the droplet with IgG expressing TZM-GFP cells. We used electrocoalescence to merge droplets, although alternative methods to merge droplets have been reported including the use of micropillar resistance arrays. The merged droplets were further incubated for another 24 hours to allow pseudovirus infection or neutralization to occur. The droplets were then broken, and the cells are recovered. Cells were allowed to recover for 48 hours. We isolated GFP- and GFP+ populations using fluorescence activated cell sorting (FACS) and extracted the gDNA from cell aliquots and performing PCR to recover the antibody gene libraries for NGS analysis. GFP-cells were also recovered and used as input for a subsequent round of screening for further enrichment for neutralizing clones.

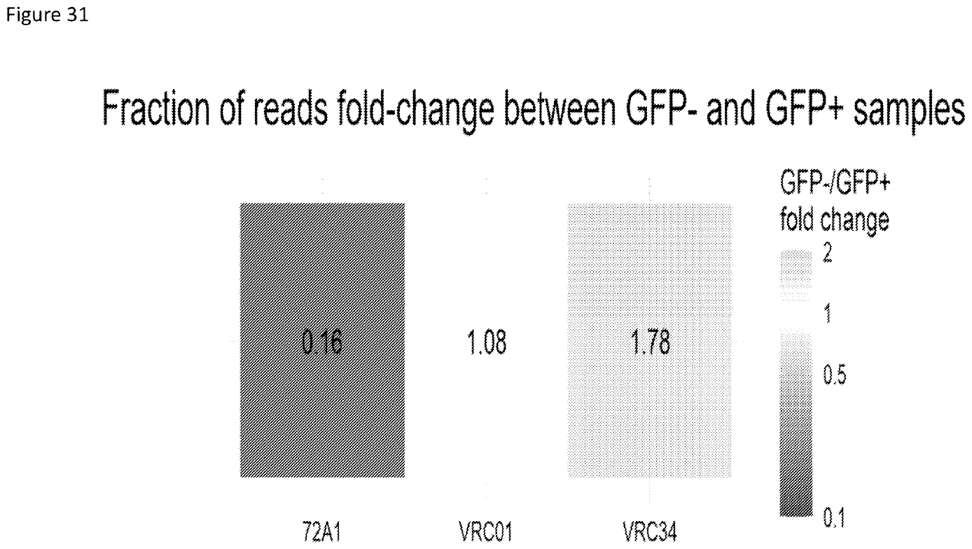

We performed high-throughput sequencing analysis on each sorted library of GFP- and GFP+ cells to obtain heavy chain sequence information. We compared the frequency of heavy chain antibody variants in each population to determine the effect of the droplet neutralization assay on neutralizing and non-neutralizing antibodies in the population (FIG. 31, Table 5).

TABLE 5
Raw sequence data and fold-change enrichment calculations for HIV-1 W6M.Env.C2
pseudovirus neutralization assays. These data demonstrate the successful
implementation of droplet neutralization assays for antibodies that neutralize
HIV-1, with NGS being used to analyze the assay performance for many
thousands of cells secreting polypeptide molecules in parallel.
HIV-1 Sort Round 1

| | | | | Fraction of | | | Fold |
|---|---|---|---|---|---|---|---|
| # reads | | | | total reads | | | change |
| Round 1 | Presort | GFP− | GFP+ | Presort | GFP− | GFP+ | GFP−/GFP+ |
| 72A1 | 408,194 | 17,476 | 58,341 | 0.07969 | 0.02121 | 0.1336 | 0.159 |
| VRC01 | 4,203,248 | 721,806 | 353,177 | 0.82062 | 0.87617 | 0.80875 | 1.083 |
| VRC34 | 510,606 | 84,538 | 25,175 | 0.09969 | 0.10262 | 0.05765 | 1.780 |
| Total | 5,122,048 | 823,820 | 436,693 | | | | |

### Example 29: Detection of Secreted Polypeptide Binding Against Cell Surface Membrane-Bound Targets

In many instances it is desired to screen a library of secreted polypeptides against cell surface membrane targets (e.g., membrane-bound targets), but effective methods to do so at scale are not readily available for all proteins. In this prophetic example, an antibody is desired against phosphatidylinositol phosphates (PIPs), a membrane-bound target, in its native, membrane-expressed form. An antibody is desired to recognize the PIPs, but the PIP does not encode for a specific signaling function and is not a signaling receptor. Prior efforts revealed methods to analyze the ability of an antibody to detect or block signaling against surface receptors, but for PIPs, no impact on signaling can be measured.

To link the secretion of a polypeptide with the recognition of PIPs, cells expressing PIPs are transformed with a library of antibodies, with each cell encoding a unique antibody polypeptide gene. The cells are compartmentalized into droplets to allow the secreted protein to secrete, and for some variants, the secreted polypeptide will bind to the cell surface if it recognizes the target. Cells are recovered from the compartments, and the secreted protein is detected if it binds to the cell surface. Those cells with positive binding events can be isolated, without regard to whether the secreted polypeptide binding event agonized or antagonized the surface target.

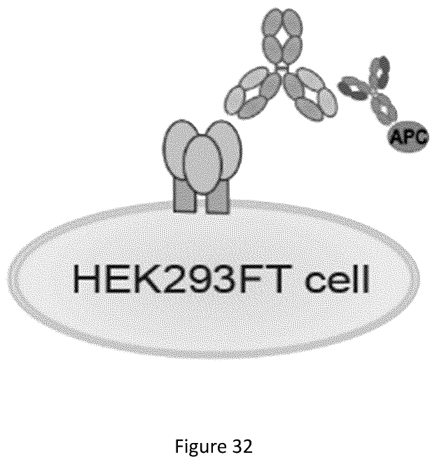

The secreted polypeptide binding event to the cell surface can be analyzed in a number of different ways, including MACS, FACS, DNA barcodes (e.g., LIBRA-seq), or DNA barcode insertion using a lentivirus. Such analysis methods are known to those skilled in the art. Sequencing of the cells after the detection event can be used to reveal the sequences of antibodies successfully bound to the cell surface target. In some embodiments, a sequencing analysis of similar cells that do not present the desired target may be used as a negative control to avoid off-target cell surface binding effects. An example of one detection system that could be used for detecting secreted polypeptide binding against cell surface membrane-bound targets is shown in FIG. 32.

In some embodiments, the cell population may be sorted to eliminate any secreted polypeptide variants in the library with off-target effects (negative selection), prior to inducing or activating the expression with the desired target for another round of screening (positive selection) In other embodiments, the recognition target may be a protein encoded by the cell. It may be a receptor, but screening for agonism or antagonism of the receptor may not be possible or may not be desired in the cell line, and thus previous approaches would not work. The target may also be a virus-derived membrane protein, as shown in FIG. 32. The method described here is effective for screening for the binding of secreted polypeptides against cell surface targets, including proteins.

### Example 30: Screening for Antibodies with Fc Protein Engagement Functions

In many instances it is desired to screen a library of polypeptides for their ability to engage Fc protein effector functions, but effective methods to do so at scale are not readily available. In this prophetic example, an antibody is desired against the HIV-1 trimer protein to activate Fc effector engagement functions via FcgRIIa.

To link the secretion of antibody with FcgRIIa-activation against the HIV-1 trimer, cells expressing a BG505-SOSIP HIV-1 trimer are transformed with a library of antibodies, with each cell encoding a unique antibody gene. The cells are compartmentalized into droplets to allow the antibody genes to secrete and accumulate in droplets, and for some polypeptide variants, the secreted antibody polypeptide will bind to the cell surface if it recognizes the HIV-1 protein target. Cells are recovered from the compartments, and the cells are stained with a soluble form of FcgRIIa. Those cells with positive FcgRIIa binding events can be isolated and sequenced.

The FcgRIIa protein binding event to the cell surface can be analyzed in a number of different ways, including MACS, FACS, DNA barcodes (e.g., LIBRA-seq), or DNA barcode insertion using a lentivirus or other vector. Such analysis methods are known to those skilled in the art

Sequencing of the cells after the detection event can be used to reveal the sequences of antibody polypeptides successfully engaging Fc effector functions or binding FC effector proteins. In some embodiments, a sequencing analysis of similar cells that do not express the desired target may be used as a negative control to avoid off-target cell surface binding effects. In some embodiments, the cell population may be sorted to eliminate any secreted protein variants in the library with off-target binding (negative selection), prior to inducing or activating the expression with the desired target for another round of screening (positive selection).

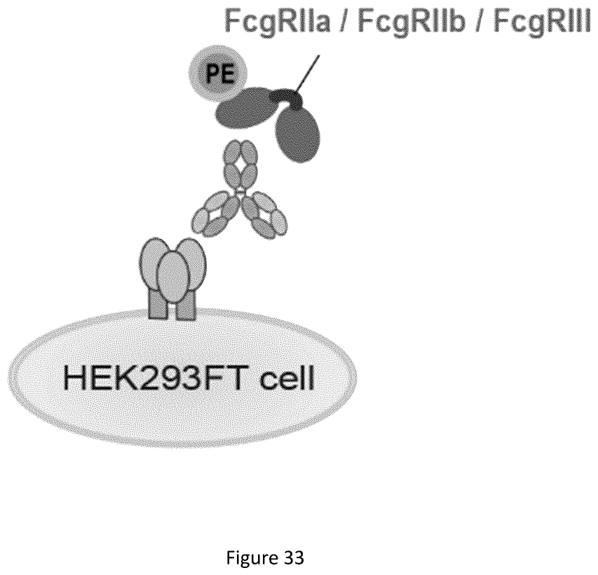

In some embodiments, the recognition target may be a polypeptide encoded by the cell. It may be a receptor, but screening for agonism or antagonism of the receptor may not be possible or may not be desired in the cell line, and thus previous approaches would not work. An example of one detection system that could be used for detecting secreted polypeptide Fc effector protein engagement is shown in FIG. 33. The polypeptide membrane-bound target may also be a virus-derived membrane protein, as shown in FIG. 33. The method described here is effective for screening for the antibody-based activation of Fc effector functions or determining Fc effector protein binding against polypeptides recognizing targets bound to a cell surface.

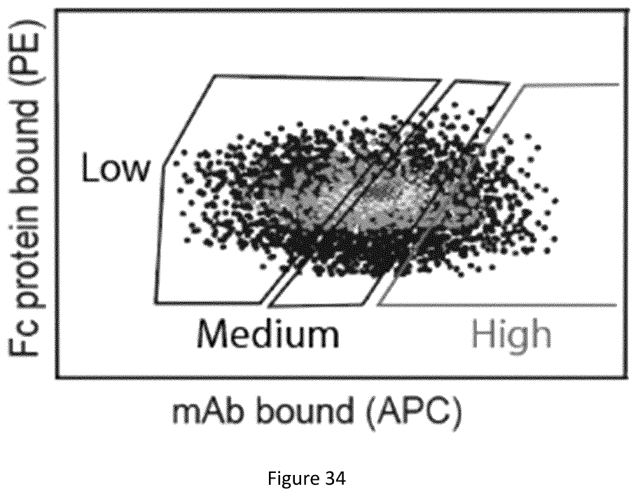

In some embodiments, the Fc effector protein engagement molecule may be an FcgR, FcaR, FcmR, or FceR. In some embodiments, the Fc effector protein engagement molecule may be in a monomeric form. In other embodiments, the Fc effector protein engagement molecule may be in a dimeric form. In some embodiments, a portion of an Fc effector protein engagement molecule may be fused to another chemical moiety or protein. Once recovered, cells with differing levels of Fc protein engagement activity may be sorted via FACS, as shown in FIG. 34, or via other cell sorting methods.

### Example 31: Screening for Antibodies with Complement Engagement Functions

In many instances it is desired to screen a library of polypeptides for their ability to engage complement functions, but effective methods to do so at scale are not readily available. In this prophetic example, an antibody polypeptide is desired against the HIV-1 trimer protein to activate complement functions.

To link the secretion of polypeptides with complement protein engagement against the HIV-1 trimer, cells expressing a BG505-SOSIP HIV-1 trimer are transformed with a library of antibodies, with each cell encoding a unique antibody polypeptide gene. The cells are compartmentalized into droplets to allow the antibody genes to secrete and accumulate in droplets, and for some variants, the secreted polypeptide will bind to the cell surface if it recognizes the HIV-1 protein target. Cells are recovered from the compartments, and the cells are stained with a soluble form of C1q. Those cells with positive C1q binding events can be isolated and sequenced.

The C1q protein binding event to the cell surface can be analyzed in a number of different ways, including MACS, FACS, DNA barcodes (e.g., LIBRA-seq), or DNA barcode insertion using a lentivirus. Such analysis methods are known to those skilled in the art.

Sequencing of the cells after the detection event can be used to reveal the sequences of polypeptides successfully engaging complement functions. In some embodiments, a sequencing analysis of similar cells that do not express the desired target may be used as a negative control to avoid off-target cell surface binding effects. In some embodiments, the cell population may be sorted to eliminate any secreted polypeptide variants in the library with off-target binding (negative selection), prior to inducing or activating the expression with the desired target for another round of screening (positive selection).

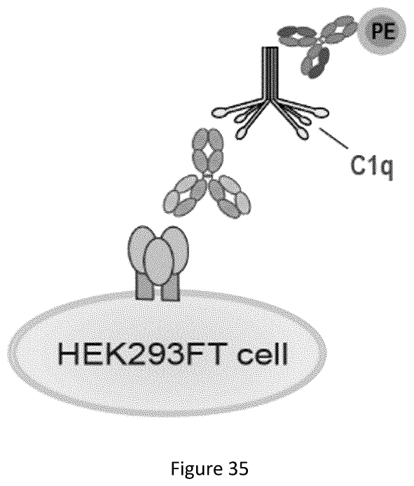

In other embodiments, the recognition target may be a protein encoded by the cell. It may be a receptor, but screening for agonism or antagonism of the receptor may not be possible or may not be desired in the cell line, and thus previous approaches would not work. The target may also be a virus-derived membrane protein, as shown in FIG. 35, or a native membrane protein, bacterial protein, nucleic acid, or chemical moiety. The method described here is effective for screening for the polypeptide-based activation of complement functions against targets bound to a cell surface.

In some embodiments, the complement engagement molecule may be any combination of C1q, C1r, and C1s. In some embodiments, the complement protein engagement molecule may be in a monomeric form. In other embodiments, the complement engagement molecule may be in a dimeric form. In some embodiments, a portion of the complement polypeptide engagement molecule may be fused to another chemical moiety or protein.

### Example 32: Screening for Polypeptide:Target Interactions at Very High Throughput

For many applications it is desired to screen for polypeptide:target interactions at very high throughput in biology. In this prophetic example, we will show the analysis of antibody:antigen libraries to deconvolute the sequence pairing of antibodies with their antigen targets at very large scale. This application is desired for many reasons, including for the parallel mapping of many antibody gene variants against other target gene variants (for example against diverse HIV-1 or SARS-CoV-2 strains or other expanded viral families), and for the rapid analysis of naturally encoded antibodies from a human individual or patient against a panel of antigens to identify antibody responses. In particular, the high-throughput polypeptide:target library screening method may be useful for identifying the targets of antibody repertoire binding against libraries of membrane proteins (e.g., membrane-bound targets) for diagnostic and research purposes.

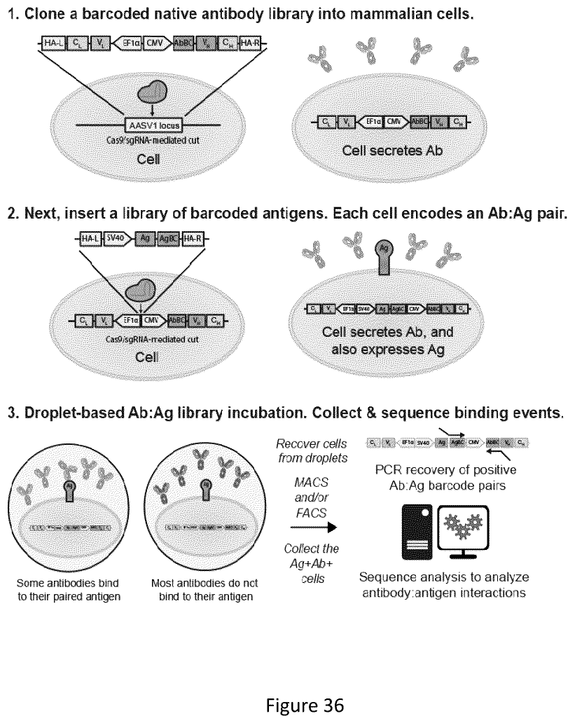

In this example, the antibody polypeptide library is first cloned into the cell using CRISPR/Cas9 targeting homology regions at a safe harbor expression locus. Next, the cells are expanded, and an antigen polypeptide library is then inserted adjacent or in between the antibody expression locus. Thus, the pairing between antibody and antigen libraries is combinatorial, and screening steps can be used to reveal the identities of the positive interacting pairs. The cells are compartmentalized, and in their compartments the cells secrete antibody. If the secreted protein (antibody in this case) binds to the cell surface, that binding event can be detected either inside the compartments, or once cells are recovered from the compartments. One possible embodiment of steps for implementation of the high-throughput protein-target interaction analysis is shown in FIG. 36.

In some embodiments, the secreted polypeptide library is cloned into the cells first, followed by a membrane bound polypeptide library (e.g., membrane-bound target library). In other embodiments, the membrane bound target library is cloned first, followed by the secreted polypeptide library. In some embodiments, the randomization of secreted and membrane polypeptides first occurs using recombinant DNA techniques, and later both secreted and membrane polypeptide gene pairs are cloned into cells together.

Gene insertion into cells can be performed in several ways known to those skilled in the art. In some embodiments, these may include lentiviruses, transient transfections, CRISPR/Cas9, integrases, transposons, and other means of inserting genes into cells known to those skilled in the art.

The present example describes the use of mammalian cells. In other embodiments, bacterial, insect, fungal, or other cells may also be used. One method of implementation would use MACS to isolate cells with polypeptides bound to their cell surface presenting polypeptide targets in early screening rounds, enabling the facile selection of positive pairwise interactions from 10⁸ and up to 10⁹ cells in a single afternoon. In some embodiments, between 100 and 10¹⁴ cells may be analyzed in parallel. Such an approach easily enables high-fidelity screening of more than 100,000 polypeptides against 1,000-10,000 or more target polypeptides per experiment, easily performed by a single researcher without robotic assistance or even a multichannel pipette. PCR amplification of polypeptide:target barcodes can reveal >50 million positive interactions on an Illumina NextSeq, and future iterations of sequencing technology will enable the analysis of many more positive interactions as sequencing yield continues to improve.

Some embodiments may select cells with pairwise interactions in 2-3 rounds of MACS, followed by one round of positive selection using FACS for high-precision data (a fast FACS experiment requiring just a few million cells).

The pairwise interaction event between the secreted polypeptide and the cell surface target can be catalogued in a number of different ways, including MACS or FACS sorting followed by sequencing of the paired genes or barcode combinations, or by the transcriptomic sequencing of DNA/RNA barcodes. Such analysis methods are known to those skilled in the art.

Some embodiments may use these technologies to map the function of antibody immune libraries on a very large scale. Table 6 is an overview of in-droplet assays for multi-feature mapping of monoclonal antibody performance. Renewable/immortalized antibody libraries provide a cutting-edge solution for multi-dimensional information from human clinical samples because they enable repeated screening and analyses under multiple conditions, including the approaches described here, to explore the many different features of antibody adaptive immune performance.

TABLE 6

| Analysis | Knowledge Gained | Technique Description |
|---|---|---|
| Antigen binding | Antigen target mapping | Antibody × Antigen libraries. TCR × pMHC libraries. |
| | | Extracellular domains as surface attachments; |
| | | membrane proteins expressed natively. |
| Epitope/domain | Molecular binding site | Subdomain antigen probes, homology domain swaps (e.g., the |
| specificity | determination | RSC3 for HIV-1 CD4bs), designed point mutations |
| Polyreactivity | Specificity / autoimmune | Polyreactive control antigen expression on cell surface; |
| | potential | antigen-null expression controls |
| Chemical moiety | Specificity / autoimmune | Surface attachment to link DNA-barcoded chemicals of interest; |
| reactivity | potential | screen as separate wells (~100); combine for NGS |
| Fc effector protein | Fc effector engagement / | FcgR (including FcgRI, FcgRIIa, FcgRIIb, FcgRIII as separate assays) |
| engagement | cell recruitment | binding signal compared to quantity of bound IgG, other Fc effector |
| | | protein assays |
| C1q engagement | Complement activation | C1q binding signal, or C1q binding signal compared to quantity of |
| | | bound IgG |
| Virus neutralization | Antiviral properties | Single-pseudovirus assays for single viruses; In-drop assays |
| | | with DNA-barcoded lentiviral particles for massively parallel |
| | | neutralization data for multiple viruses concurrently |

In some embodiments, the screening assay may also bin polypeptides for multi-variate features like multiple Fc effector engagement assays, and/or complement assays for comparative evaluation via sequencing. In addition to pairwise analysis, some embodiments may use directed evolution to engineer antibodies against broad antigen panels and escape variants, which is needed for diverse & evolving viruses like coronaviruses, HIV-1, flaviviruses, and others.

In some embodiments, a chemical may be associated with either the membrane-bound target or the secreted polypeptide. In some embodiments, the chemical may be barcoded, for example using a DNA sequence (see e.g., Gironda-Martinez A, Donckele E J, Samain F, Neri D. DNA-Encoded Chemical Libraries: A Comprehensive Review with Successful Stories and Future Challenges. ACS Pharmacol Transl Sci. 2021 Jun. 14; 4(4):1265-1279. doi: 10.1021/acsptsci.1c00118. PMID: 34423264; PMCID: PMC8369695). In some embodiments, a polypeptide may be barcoded and associated with either the membrane-bound target or the secreted polypeptide, for example using a DNA sequence (see e.g., Stoeckius M, Hafemeister C, Stephenson W, Houck-Loomis B, Chattopadhyay P K, Swerdlow H, Satija R, Smibert P. Simultaneous epitope and transcriptome measurement in single cells. Nat Methods. 2017 September; 14(9):865-868. doi: 10.1038/nmeth.4380. Epub 2017 Jul. 31. PMID: 28759029; PMCID: PMC5669064). In some embodiments, a barcode may be sequenced to reveal the identity of a chemical or a polypeptide that was associated with either the membrane-bound target or the secreted polypeptide.

Some embodiments may incorporate recently established barcoded lentiviral-based pseudovirus neutralization assays to assay antibody virus neutralization inside single-cell droplets for multiple viruses concurrently. In some embodiments, a sequencing analysis of similar cells that do not express the desired target may be used as a negative control to avoid off-target cell surface binding effects, or to analyze or eliminate autoreactivity or polyreactivity from a polypeptide library via screening.

In some embodiments, the cell population may be sorted to eliminate any secreted polypeptide variants in the library with off-target effects (negative selection), prior to inducing or activating the expression with the desired target for another round of screening (positive selection).

In some embodiments, a library of secreted polypeptide variants may be generated. In other embodiments, a library of membrane-bound targets may be generated. In some embodiments, a library of secreted polypeptide variants may be combined with a library of membrane-bound targets. In some embodiments, the two separate libraries (secreted polypeptides, and membrane-bound targets) may be encoded on the same nucleic acid strand, for example in a DNA plasmid, an mRNA, or a linear expression cassette. In some embodiments, a combinatorial library of secreted polypeptide variants crossed with membrane-bound targets may be generated by first cloning one library, and then cloning the other library. In some embodiments, a population of cells will be engineered with one library, and then engineered with the other library, to create a population of cells with a library of secreted polypeptide variants may be combined with a library of membrane-bound targets. In some embodiments, Cas9-guided nucleic acid insertion may be used to insert multiple nucleic acid cassettes into multiple safe harbor loci within a cell, and this process may generate a library of secreted polypeptide variants may be combined with a library of membrane-bound targets. Approaches to combine a library of secreted polypeptide variants with a library of membrane-bound targets present a powerful use case for this technology that enables high-throughput analysis of interactions between a library of secreted polypeptide variants and a library of membrane-bound targets. A broad variety of methods for creating gene libraries are known to those skilled in the art.

### Example 33: Validation of Polypeptide Secretion and Membrane-Bound Target Binding from Single Cells Inside Droplet Compartments

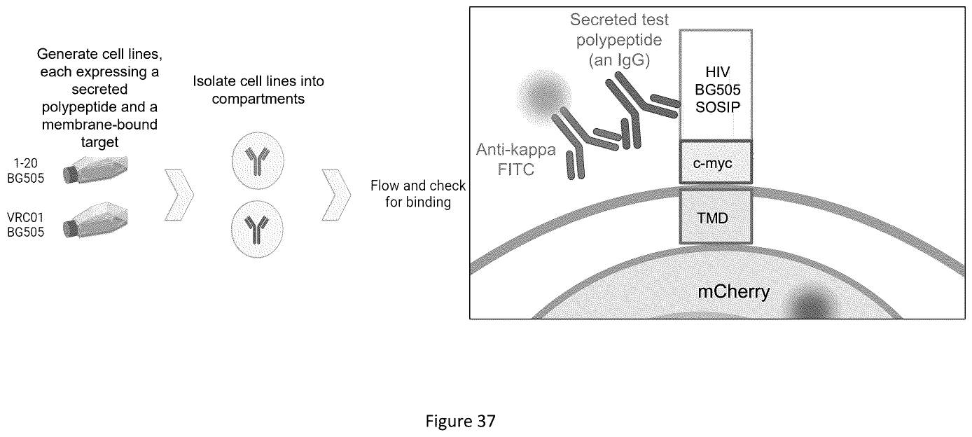

Briefly, genes encoding surface HIV-1 Env BG505 SOSIP and secreted antibodies (1-20, negative control that does not bind to HIV-1 Env, and VRCO1, a positive control that binds to HIV-1 Env), were expressed by HEK293 cells within droplets and incubated for 24 hrs. Cells were recovered and stained with anti-cmyc-AF647 and anti-Kappa-FITC, then fixed. Anti-Kappa-FITC binding was measured for cells expressing mCherry and bound by anti-cmyc-AF647 via FACS. Cells expressing an HIV-1 Env binding antibody, VRCO1, had over 5 times more anti-Kappa binding than cells expressing the HIV-1 Env non-binding antibody, 1-20 (FIGS. 37,38)^{15, 63,68}. Detailed methods for this example are provided below.

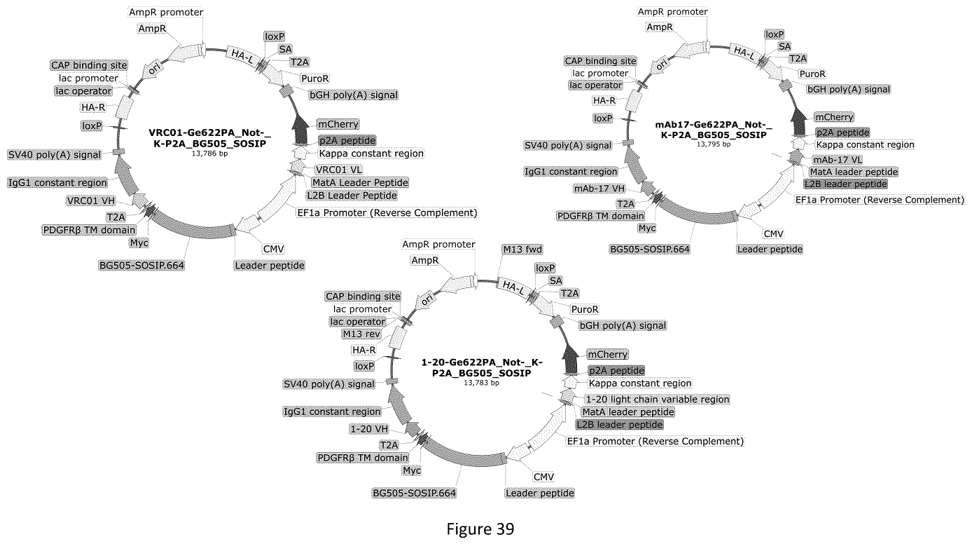

Cell line generation: A CRISPR/Cas9 system was used to generate stable cell lines expressing surface antigens (e.g., the membrane-bound target) and also secreting IgG1 antibodies (the secreted polypeptide). HEK293 adherent cells supplemented Dulbecco's modified Eagle's medium high glucose (DMEM; ThermoFisher) were seeded in a 6 well plate at 0.8*10⁶ cells/well. After incubation at 37° C. for approx. 24 hours, the cells (70-90% confluent) were co-transfected with two plasmids, GE622-DONOR (FIG. 39) and EF1-hspCas9-H1-AAVS1-gRNA.

Following the manufacturer's recommended Lipofectamine 3000 transfection protocol, 7.5 uL of Lipofectamine 3000 was added to 250 μL of OptiMEM. In another tube, 10 μL of P3000, 1 μg of the GE622-DONOR plasmid (FIG. 39) and 1 μg of EF1-hspCas9-H1-AAVS1-gRNA were added to 250 μL of OptiMEM. The contents of the two tubes were combined and incubated at room temperature for 20 minutes. The transfection mixture was then added dropwise to a well of cells. The cells were incubated at 37° C. for 3 days, and were expanded as needed to a T25, then a T75, flask.

On day 3, the cells were washed with Dulbecco's Phosphate Buffered Saline (DPBS) and detached using trypLE. After recovering the cells in supplemented DMEM and centrifuging (500×g, 5 mins) the cells to form a pellet, all of the cells were resuspended in FACS buffer (DPBS supplemented with 2 mm EDTA and 1% BSA) and sorted twice for mCherry expression via FACS on a Sony SH800S.

Droplet generation and incubation. Cultured HEK-293 BG505+1-20 and HEK-293 BG505+VRCO1 cells in a T75 flask were washed twice with DPBS and detached using trypLE. The cells were recovered in supplemented DMEM and counted using a digital counter.

Cells at a concentration of 3.2*10⁶ cells/mL were added to a syringe and connected to a microfluidic chip port with tubing. Supplemented DMEM was also placed in a syringe was connected to another port of the chip. Surfactant oil was injected into the chip at a third port. Each of the syringes were placed in a syringe pump to control flow rates. Generated single-cell droplets were collected, and the contents were placed into an upright T25 flask and incubated at 37° C. for 1 day. The next day, the droplets were broken using a destabilizing surfactant and the cells were recovered in a bulk media phase.

Staining & FACS. Cells were placed in 1.5 mL tubes and centrifuged at 300×g for 5 mins. 100 μL of 5% (diluted in FACS buffer) anti-Kappa-FITC antibody stain was added to each cell pellet. The cells were resuspended and incubated at 4° C. for 30 mins. The cells were washed twice with FACS buffer, then resuspended in 100 μL of 1% anti-cmyc-AF647 and incubated at 4° C. for 30 mins. The cells were washed in FACS buffer, then resuspended in 250 μL of FACS buffer and sorted for mCherry, AF647, and FITC expression (FIGS. 37-39).

This example demonstrates the effectiveness of determining the binding interactions for a single cell secreting a test polypeptide with a membrane-bound target. In this example, the test polypeptide (IgG1 variants) were encoded on a single plasmid along with the membrane-bound target (an HIV-1 Env variant), and the binding association between the secreted test polypeptide and the membrane-bound antigen was analyzed. In this example, a certain stringency of flow cytometry gates were selected. In practice, a user skilled in the art will adjust the stringency of flow cytometry gates in accordance with their desired parameters, and can change the selective stringency of a flow analysis in accordance with their goals.

This example could be implemented in a variety of ways, for example, encoding the secreted test polypeptide and the membrane-bound antigen on separate nucleic acid strands. In some implementations, either the test polypeptide or the membrane-bound antigen may be endogenously encoded by a cell. A variety of methods to enable polypeptide secretion, as well as membrane-bound antigen presentation, are widely known to individuals skilled in the art.

In this example, the secreted test polypeptide and the membrane-bound antigen genes were inserted into cells for expression using a Cas9-based system. In other implementations, polypeptide genes may be inserted into cells using alternative techniques that are known to those skilled in the art.

In this example, the compartment was a droplet. In other implementations, the compartment may be another type of compartment known to those skilled in the art.

### Example 34: High-Throughput Isolation and Sequence-Based Detection of Specific Antibody:Antigen Binding Pairs

Cell line generation. A CRISPR/Cas9 system was used to generate stable cell lines expressing surface antigens (the membrane-bound target) and also secreting IgG1 antibodies (the secreted polypeptide). HEK293 adherent cells supplemented Dulbecco's modified Eagle's medium high glucose (DMEM; ThermoFisher) were seeded in a 6 well plate at 0.8*10⁶ cells/well. After incubation at 37° C. for approx. 24 hours, the cells (70-90% confluent) were co-transfected with two plasmids, GE622-DONOR (FIG. 39) and EF1-hspCas9-H1-AAVS1-gRNA.

Following the manufacturer's recommended Lipofectamine 3000 transfection protocol, 7.5 uL of Lipofectamine 3000 was added to 250 μL of OptiMEM. In another tube, 10 μL of P3000, 1 μg of the GE622-DONOR plasmid (FIG. 39) and 1 μg of EF1-hspCas9-H1-AAVS1-gRNA were added to 250 μL of OptiMEM. The contents of the two tubes were combined and incubated at room temperature for 20 minutes. The transfection mixture was then added dropwise to a well of cells. The cells were incubated at 37° C. for 3 days, and were expanded as needed to a T25, then a T75, flask.

On day 3, the cells were washed with Dulbecco's Phosphate Buffered Saline (DPBS) and detached using trypLE. After recovering the cells in supplemented DMEM and centrifuging (500×g, 5 mins) the cells to form a pellet, all of the cells were resuspended in FACS buffer (DPBS supplemented with 2 mm EDTA and 1% BSA) and sorted twice for mCherry expression via FACS on a Sony SH800S.

Droplet generation and incubation. Cultured HEK-293 BG505+1-20, HEK-293 BG505+mAb17, and HEK-293 BG50+VRCO1 cells were grown separately in a T75 flask were washed twice with DPBS and detached using trypLE. The cells were recovered in supplemented DMEM and counted using a digital counter. The cells were then mixed such that the final volume of cells was 0.5% HEK-293 BG505+mAb17, 0.5% HEK-293 BG505+VRCO1, and 99% HEK-293 BG505+1-20.

Cells at a concentration of 3.2*10⁶ cells/mL were added to a syringe and connected to a microfluidic chip port with tubing. Supplemented DMEM was also placed in a syringe was connected to another port of the chip. Surfactant oil was injected into the chip at a third port. Each of the syringes were placed in a syringe pump to control flow rates. Generated single-cell droplets were collected, and the contents were placed into an upright T25 flask and incubated at 37° C. for 1 day. The next day, the droplets were broken using a destabilizing surfactant and the cells were recovered in a bulk media phase.

Staining & FACS. Cells were placed in a 1.5 mL tube and centrifuged at 300×g for 5 mins. 100 μL of 5% (diluted in FACS buffer) anti-Kappa-FITC was added to each cell pellet. The cells were resuspended and incubated at 4° C. for 30 mins. The cells were washed twice with FACS buffer, then resuspended in 100 μL of 1% anti-cmyc-AF647 and incubated at 4° C. for 30 mins. The cells were washed in FACS buffer, then resuspended in 250 μL of FACS buffer and sorted for mCherry, AF647, and FITC expression. Two populations were collected: approx. 7,000 cells that were mCherry+, AF647+, and FITC+, and approx. 34,000 cells that were mCherry+, AF647+, and FITC−.

Next Generation Sequencing (NGS) preparation and analysis. Both sort populations were collected and seeded in 96-well plates for four days. Cells were then detached using trypLE. The DNA was then extracted using a Zymo Genomic DNA Purification Kit. A PCR (ThermoFisher Platinum Taq DNA Polymerase kit) was performed to amplify the variable heavy chain antibody gene from the genomic DNA, using primers specific to the region. The PCR product was separated and purified using gel extraction.

Next, two additional PCRs (Kapa HiFi HotStart ReadyMix) were run using MiSeq1 and MiSeq2 primers to prepare for Next Generation Sequencing (NGS). DNA quality was then assessed using a Qubit Fluorometer and a 4200 TapeStation instrument. The DNA was then submitted for NGS analysis using an Illumina MiSeq.

NGS reads were pre-processed to combine reads 1 and 2 using FLASH, and then merged reads were quality filtered using fastx_toolkit with settings of q30 and p50. NCBI IgBlast was used to analyze V-genes and CDR3 sequences. Antibodies were mapped using VH gene and CDR3 sequence (100% identity). The number of reads for each clone in FITC- and FITC+ populations were compiled, and enrichment ratios were calculated to detect sorting & enrichment signals for known positive binding pairs (Table 7).

After only a single round of sorting, an enrichment ratio greater than 1.01 was indicative of a positive interacting pair. These data demonstrate the ability to screen libraries of antibody:antigen pairs for positive binding interactions in a high-throughput, library:library format by capturing single cells expressing both a secreted test polypeptide and a membrane-bound target within a compartment.

In some implementations, the libraries of cells may be recovered after a sort, expanded, sorted, and sequenced again to further improve the enrichment ratio, as is common in library screening experiments such as phage display, yeast display, and mammalian cell display. In some implementations of this workflow, the flow cytometry gates may also be set more stringently to enhance the purity of selection in each round. Thus, the observed enrichment ratios can vary with the selective stringency of sort conditions and the number of rounds of screening performed, enabling an end user to optimize the library:library screening conditions according to desired use. Various additional methods for adjusting sort conditions and sort stringency in each round, for repeating screenings across multiple rounds, and for analyzing selection performance and efficiency can be exercised by individuals skilled in the art.

In this example, a synthetic library was generated with three different IgG1 clones as the secreted test polypeptide, each with a membrane-bound target of the HIV-1 BG505.DS-SOSIP trimeric antigen. Here, the secreted test polypeptide (IgG1) and the membrane-bound target (HIV-1 BG505.DS-SOSIP trimeric antigen) were encoded on the same plasmid. The plasmid was integrated into the genome via co-transfection of the single plasmid containing the secreted test polypeptide and the membrane-bound target, along with and a plasmid encoding gRNA/Cas9. Each of the three cell lines were mixed together at a ratio of approximately 99% 1-20, 0.5% mAb-17, and 0.5% VRCO1. The cells were sorted, lysed, and evaluated via next-generation sequencing. The results of the sorting and enrichment are shown in Table 7. After a single round of sorting, an enrichment ratio greater than 1.01 was indicative of a positive interacting pair. These data demonstrate the ability to screen libraries of antibody:antigen pairs for positive binding interactions in a high-throughput, library:library format by capturing single cells expressing both a secreted test polypeptide and a membrane-bound target within a compartment.

TABLE 7
Evaluation of next-generation sequencing data after
sorting for a test antibody:antigen library.

| mAb clone | | | | | Enrichment Ratio (FITC+ |
|---|---|---|---|---|---|
| (secreted test | # reads in | # reads in | Fraction of | Fraction of | prevalence / FITC− |
| polypeptide) | FITC− | FITC+ | FITC− | FITC+ | prevalence) |
| mAb 1-20 | 312,568 | 94,129 | 0.992 | 0.999 | 1.0 |
| mAb-17 | 2,324 | 1 | 0.00738 | 0.0000106 | 0.0014 |
| VRC01 | 138 | 81 | 0.000438 | 0.000860 | 2.0 |
| Total reads | 315,030 | 94,211 | | | |

### Example 34: Validation Target Antigen Binding by Cell-Secreted Polypeptides in the Form of IgG1 Monoclonal Antibodies

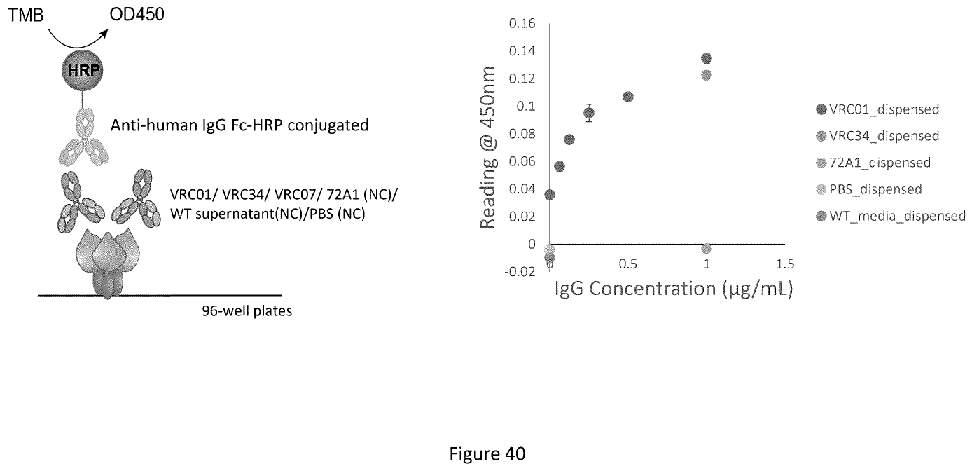

We tested the binding between a soluble HIV-1 trimer and monoclonal antibodies secreted by cells. After cells secreted IgG concentration in the form of secreted test polypeptides, the IgG concentration was calculated by anti-p2A ELISA to determine the concentration of IgG in the supernatant. Next, ELISA plate coating was conducted with 5 μg/mL of P024-A.Q842.d12_DS-SOSIP protein, in 100 μl of 0.1M NaHCO₃ in a 96-well flat-bottom Nunc-Immuno Plate (Thermo Scientific, IL) overnight at 4° C., followed by the removal of the coating solution and three washes with wash buffer (PBS—0.01% Tween 20). Then the wells were blocked by incubation of the wells with 100 μl of 3% (w/v) BSA in phosphate-buffered saline (PBS) for 1 hr at room temperature. The wells were washed three times with 200 uL wash buffer (PBS—0.01% Tween 20). One hundred microliters of 1 μg/mL TZM-GFP cell secreted monoclonal IgG1 antibodies⁶¹: VRCO1 (an HIV-1-specific antibody)^{62,63} VRC34.01 (an HIV-1 specific antibody)^{64,65}, and 72A1 (a control antibody that does not bind to HIV Env)^{66,67}, were added to designated wells at specified concentrations and incubated for 1 hr at 37° C., after which the wells were washed three times with wash buffer. One hundred microliters of horseradish peroxidase (HRP)-conjugated goat anti-human IgG antibody (Thermo Scientific, IL) at 1:4,000 in PBS with 1.0% (w/v) BSA and 0.1% Tween 20 was added to each well and incubated for 1 hr at RT, after which the wells were washed five times with PBS—0.01% Tween 20. The wells were developed with tetramethylbenzidine (TMB) at room temperature for 10 min, after which the reaction was stopped with 180 mM HCl. The readout was measured at a wavelength of 450 nm. All samples were read in triplicates (FIG. 40). These data demonstrate that known IgG1 monoclonal antibodies can efficiently bind their known target antigens when expressed in the form of secreted test polypeptides.

### Example 35: Validation of Secreted Control Polypeptides Recruiting Fc Effector Proteins

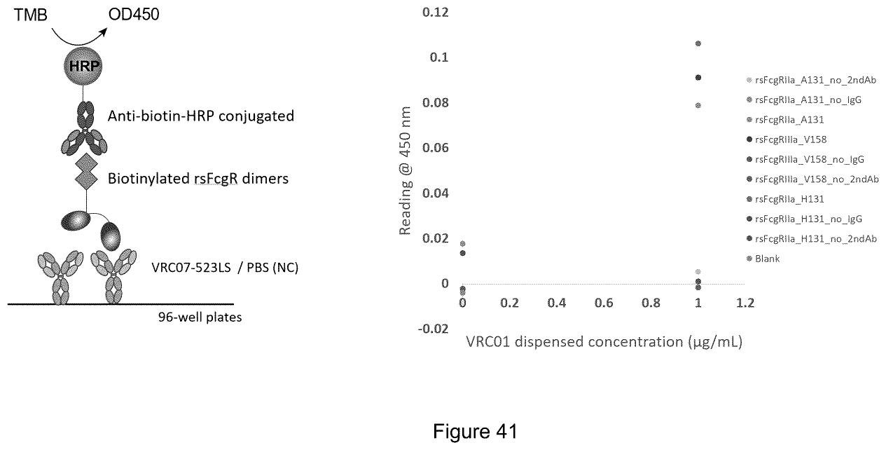

This example validated the presence of binding between known recombinant soluble Fc gamma receptor (rsFcgR) detection agents and test polypeptide monoclonal antibodies secreted by cells. After cells secreted IgG concentration in the form of secreted test polypeptides, the IgG concentration was calculated by anti-p2A ELISA to determine the concentration of IgG in the supernatant. Next, ELISA plates were coated with 1 μg/mL of mAbs: VRCO1, VRC34.01, and VRC07-523LS⁶² in a 96-well flat-bottom Nunc-Immuno Plate (Thermo Scientific, IL) overnight at 4° C., followed by the removal of the coating solution and three washes with wash buffer (PBS-0.05% Tween 20). The wells were then blocked with 100 μl of 1% (w/v) BSA in PBS-0.05% Tween 20 for 1 hr at RT, after which the wells were washed three times with wash buffer. One hundred microliters of biotinylated recombinant human dimeric FcgRs: rsFcgRIIa_A131, rsFcgRIIa_R131, and rsFcgRIIIa_V158 were added to designated wells at concentration of 0.5 μg/mL in PBS-0.05% Tween 20 with 1% (w/v) BSA for 1 hr at 37 C, followed by five washes. One hundred microliters of HRP-conjugated mouse anti-biotin antibody (Thermo Scientific, IL) at 1:1,000 in PBS with 1.0% (w/v) BSA and 0.05% Tween 20 was added to each well and incubated for 1 hr at room temperature, after which the wells were washed five times with PBS—0.01% Tween 20. The wells were developed with TMB at room temperature for 10 min, after which the reaction was stopped with 180 mM HCl. The readout was measured at a wavelength of 450 nm. All samples were read in triplicates (FIG. 41). These data demonstrate that IgG1 monoclonal antibodies expressed by cells in the form of secreted test polypeptides can bind to Fc gamma receptor proteins that related to antibody Fc effector immune responses.

### Example 36: Validation of Secreted Control Polypeptides Binding to Membrane-Bound Targets

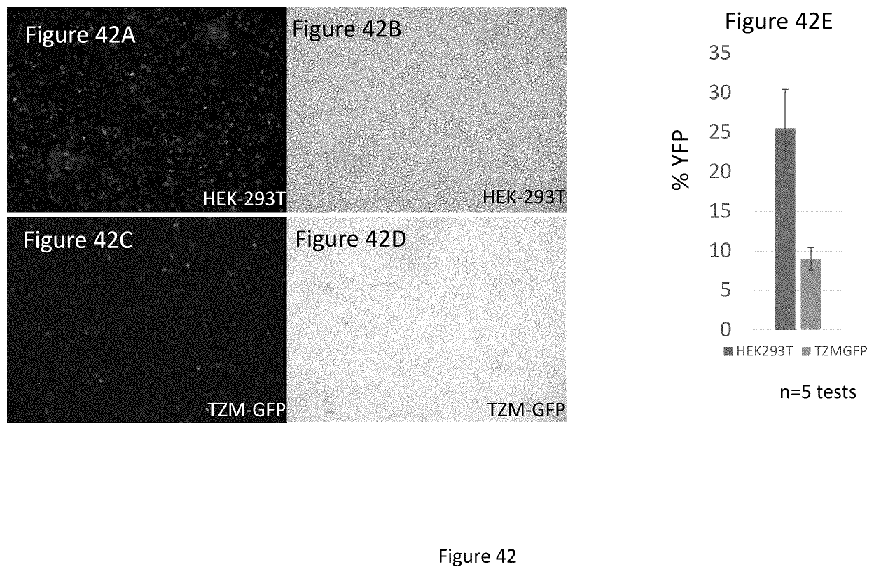

A cell-surface HIV-1 trimer antibody binding assay was performed using cells expressing a BG505.SOSIP trimer antigen. This experiment evaluated the ability of cells to secrete a polypeptide (IgG1 antibody) that would bind appropriately to a membrane-bound target (a BG505.SOSIP trimer antigen). In 6-well flat-bottom tissue-treated plates (Thermo Scientific, IL), two different cell lines (HEK-293T and TZM-GFP) were engineered to secrete a panel of antibodies (HEK-293T VRC07-523LS, TZM-GFP_VRC01, and TZM-GFP VRC34.01), or were cultured as control cells (WT). In some groups, antibody-expressing cells were transfected with BG505.SOSIP-6R-TM-YFP (synthesized via Genscript, Piscataway, NJ) using Lipofectamine3000 (Thermo Scientific, IL) to induce surface expression (FIG. 42). Cells were also incubated for 48 hrs at 37° C., after which the supernatant was collected for subsequent antibody concentration measurement with anti-p2A ELISA.

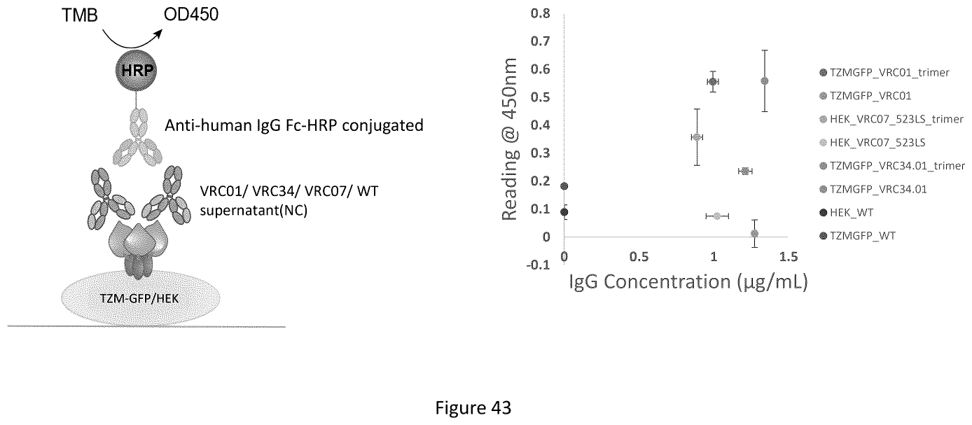

The cells were mechanically detached, washed two times with 10 mL of PBS, and evenly distributed as 1 million cells per sample in 1.5 mL Eppendorf tubes. Each sample was blocked with PBS-0.01% Tween 20 with 2% (w/v) BSA for 1 hr at 37° C., after which the cells were washed three times with 1 mL PBS. Two hundred microliters of horseradish peroxidase (HRP)-conjugated goat anti-human IgG Fc antibody (Thermo Scientific, IL) at 1:4,000 in PBS with 1.0% (w/v) BSA and 0.01% Tween 20 was added to each sample and incubated for 1 hr at room temperature, after which the cells were washed three times with 1 ml PBS. Then, each sample was fixed with 250 uL Fixation Buffer (BioLegend, San Diego, California) and left in the dark for 20 minutes at room temperature. The samples were developed with TMB at room temperature for 10 min, after which the reaction was stopped with 180 mM HCl. The readout was measured at a wavelength of 450 nm. All samples were read in triplicates (FIG. 43). The markedly higher 450 nm absorption signals from wells containing cells secreting antibody and expressing BG505.SOSIP trimer concurrently, compared to lower signals for WT cells or cells expressing antibody alone (no BG505.SOSIP trimer) demonstrated that cells can both express a membrane-bound target (BG505.SOSIP trimer) and concurrently capture secreted polypeptides (IgG1) in a format that enables cell isolation and high-throughput secreted polypeptide binding assays for recognition of desired cell surface targets.

### Example 37: Validation of Secreted Control Polypeptides Binding to Membrane-Bound Targets and Recruiting Fc Effector Proteins

In 6-well flat-bottom tissue-treated plates (Thermo Scientific, IL), HEK293_VRC07-523LS, TZMGFP_VRCO1, TZMGFP_VRC34.01, and TZMGFP_72A1 cells were transfected with BG505.SOSIP-6R-TM-YFP (synthesized via Genscript, Piscataway, NJ) using Lipo3000 (Thermo Scientific, IL) for surface expression and incubated for 48 hrs in 37 C, after which the supernatant was collected for subsequent antibody concentration measurement with p2A ELISA. The cells were mechanically detached, washed two times with 10 mL of PBS, and evenly distributed as 1M cells per sample in 1.5 mL Eppendorf tubes. Biotinylated recombinant human dimeric FcgRs: rsFcgRIIb, rsFcgRIIa_R131, and rsFcgRIIIa_V158 were added to each sample at concentration of 0.5 μg/mL in PBS-0.05% Tween 20 with 1% (w/v) BSA for 1 hr at 37° C., after which the samples were washed two times with PBS. Then, each sample was blocked by PBS—0.01% Tween 20 with 2% (w/v) BSA for 1 hr at 37° C., after which the cells were washed two times with 1 mL PBS.

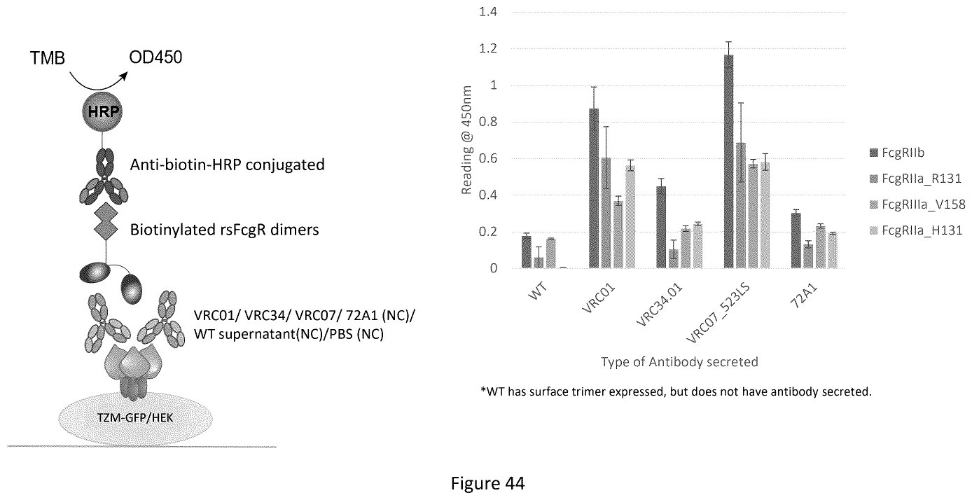

Two hundred microliters of horseradish peroxidase (HRP)-conjugated mouse anti-biotin antibody (Thermo Scientific, IL) at 1:4,000 in PBS with 1.0% (w/v) BSA and 0.01% Tween 20 was added to each sample and incubated for 1 hr at room temperature, after which the cells were washed three times with 1 mL PBS. Then, each sample was fixed with 250 uL Fixation Buffer (BioLegend, San Diego, California) and left in the dark for 20 minutes at room temperature. The samples were developed with TMB at room temperature for 10 min, after which the reaction was stopped with 180 mM HCl. The readout was measured at a wavelength of 450 nm. All samples were read in triplicates (FIG. 44). The markedly higher 450 nm absorption signals from wells containing cells secreting antibody and expressing BG505.SOSIP trimer concurrently, compared to lower signals for WT cells or cells expressing a non-HIV binding antibody demonstrated that cells can both express a membrane-bound target (BG505.SOSIP trimer) and concurrently capture secreted polypeptides (IgG1) that then recruit FcgR proteins, thereby enabling cell isolation into a compartment and high-throughput secreted polypeptide binding assays for Fc effector protein recruitment against membrane-bound targets.

Of note, one of the antibodies (VRC34.01), was observed to recruit FcgR proteins less efficiently than the other HIV-1 specific antibodies (VRCO1, VRC07-523LS), even though VRC34.01 was efficiently secreted by the cells (FIG. 43). Fc effector function is known to show diversity for different antibodies against the same target antigen, which can occur based on differences in the variable region or the constant region^{69,70}. This example demonstrates the capability to achieve different levels of Fc effector recruitment for secreted polypeptides containing Fc regions, even when binding to the same membrane-bound target. The inventions described here thus permit the high-throughput screening and analysis of secreted polypeptides based directly on the ability of a secreted polypeptide to recruit Fc effector proteins.

### Example 38: Validation of Secreted Control Polypeptides Binding to Membrane-Bound Targets Inside Droplets with Staining for Recruiting Fc Effector Proteins

This example demonstrates the ability to isolate single cells inside compartments (droplets) secreting a test polypeptide (IgG1) specific to a membrane-bound target (HIV-1 Env), and detecting that interaction using Fc gamma effector proteins.

Cultured HEK293_VRC07-523LS and TZMGFP_VRC01 cells with surface HIV-1 Env trimer expression (B0505.SOSIP-6R-TM-YFP) were grown separately in a 6-well flat-bottom plates and were washed twice with DPBS and mechanically detached using cell scraper. In some experiments, TZM-GFP cells expressing HIV-1 Env protein were also used as a control, without expressing IgG1. The cells were recovered in supplemented DMEM and counted using a digital counter.

Cells at a concentration of 2*10⁶ cells/mL were added to a syringe and connected to a microfluidic chip port with tubing. Supplemented DMEM was also placed in a syringe was connected to another port of the chip. Surfactant oil was injected into the chip at a third port. Each of the syringes were placed in a syringe pump to control flow rates. Generated single-cell droplets were collected, and the contents were placed into 6-well flat-bottom plates and incubated at 37° C. for 1 day. The next day, the droplets were broken using a destabilizing surfactant and the cells were recovered in a bulk media phase.

The cells were washed two times with 10 mL PBS, after which the biotinylated recombinant human dimeric FcgRIIa_R131, FcgRIIb, or FcgRIIIa_V158 was added to each sample as indicated at concentration of 0.5 μg/mL in PBS-0.05% Tween 20 with 1% (w/v) BSA for 1 hr at 37° C. Samples were washed two times with PBS. Then, each sample was blocked by PBS-0.01% Tween 20 with 2% (w/v) BSA for 1 hr at 37° C., after which the cells were washed two times with 1 mL PBS.

Two hundred microliters of mouse anti-biotin PE antibody (Thermo Scientific, IL) at 1:1,000 in PBS with 1.0% (w/v) BSA and 0.01% Tween 20 was added to each sample and incubated for 1 hr at room temperature, after which the cells were washed three times with 1 mL PBS. Then, each sample was fixed with 250 μL Fixation Buffer (BioLegend, San Diego, California) and left in the dark for 20 minutes at room temperature. The cells were washed in FACS buffer, then resuspended in 250 IL of FACS buffer and analyzed for PE signal and YFP expression.

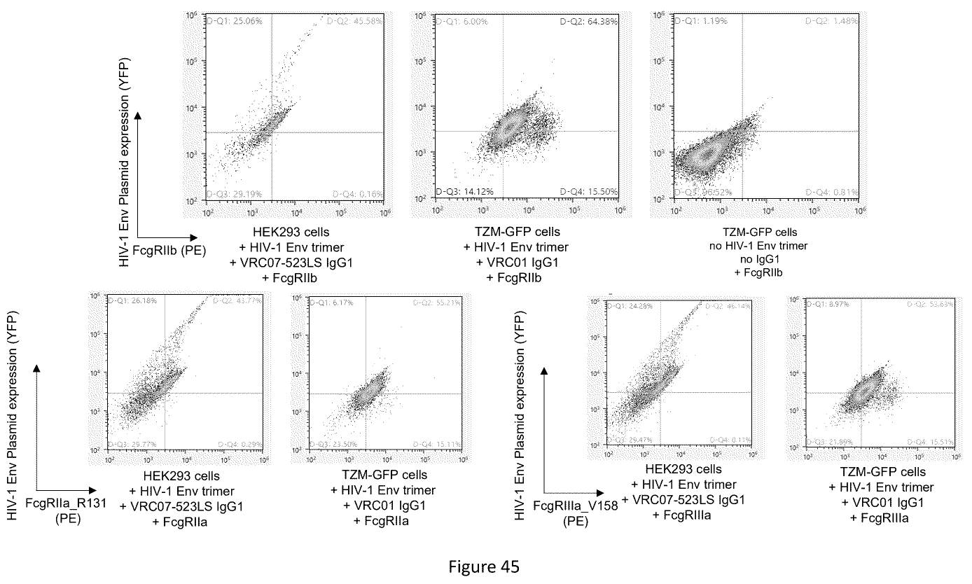

After compartmentation and recovery from the droplets, cells were appropriately stained with recombinant Fc effector protein that detected the association between the secreted test polypeptide and the membrane-bound target (FIG. 45). The interaction between the secreted polypeptide and the membrane-bound target was observed for multiple cell types used (both HEK-293, and TZM-GFP cells). The interaction was also detected successfully using multiple Fc effector proteins (FcgRIIa_R131, FcgRIIb, and FcgRIIIa V158). This example thus demonstrates the use of a single compartment for high-throughput screening of antibody-antigen interactions that lead to the recruitment of Fc effector proteins.

### Example 39: Analysis of T Cell Receptor and pMHC Libraries

In this prophetic example, we perform the mapping of T cell receptor (TCR) and peptide:MHC (pMHC) libraries using a high-throughput screening approach. Using a single plasmid encoding both control TCRs as a secreted polypeptide, and control pMHCs as a membrane-bound target, a cell is engineered by co-transfection of the plasmid containing the secreted test polypeptide and the membrane-bound target, along with and a plasmid encoding gRNA/Cas9. The secreted polypeptide and the membrane-bound targets are thus integrated into the genome of each single cell. In some cases, the secreted polypeptide is a pMHC, and the membrane-bound target is a TCR. In some cases, the secreted polypeptide and the membrane-bound antigen are encoded on different plasmids. In some cases, the secreted polypeptide and the membrane-bound antigen are encoded on linear nucleic acid sequences for genetic library insertion into a cell. In some cases, the secreted polypeptide and the membrane-bound antigen are combined together as a single nucleic acid sequence for genetic library insertion into a cell.

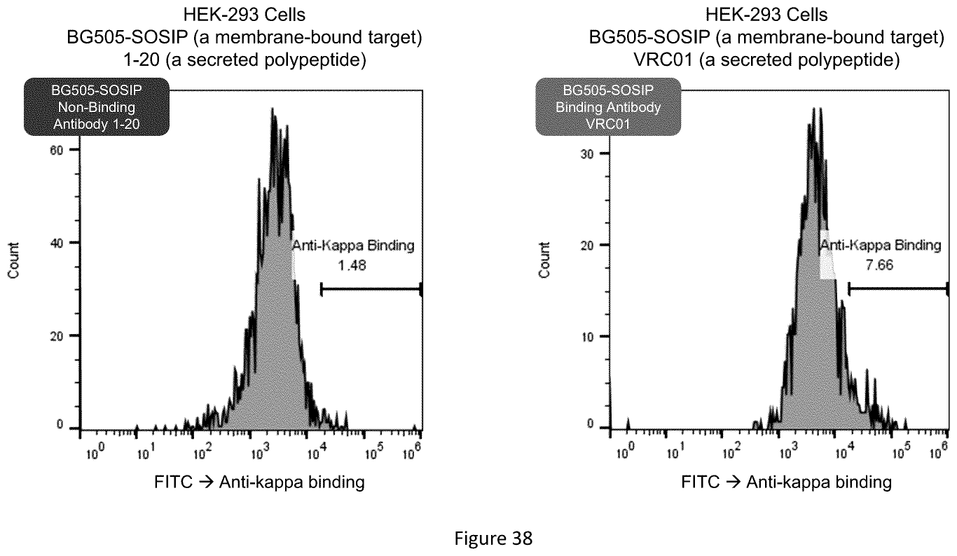

In this example, the cell is isolated inside a compartment. Binding associations are detected between the secreted test polypeptide (a soluble TCR) to the membrane-bound target (a pMHC) by staining cells using a conjugated antibody to detect the presence of bound secreted test polypeptides, as shown in FIG. 38 and FIG. 43. The mechanics of a library:library analysis using a secreted test polypeptide and membrane-bound antigen with TCR-pMHC interactions works in the same manner as the antibody-antigen interactions described in Examples 32-38. The cell is isolated by flow cytometry or by magnetic sorting, and the gene encoding the membrane-bound target and the secreted polypeptide are sequenced and identified. In some cases, the enrichment ratio between cells with bound secreted test polypeptides vs. cells without bound secreted test polypeptides is analyzed, and an enrichment of gene pairs in the population of cells with bound secreted test polypeptides is detected and is indicative of a positive binding interaction; sequence data may be collected as shown in Table 7. In this manner, the diverse pMHC binders for to a large set of T cell receptors can be identified using a library of pMHC antigens, greatly aiding in the diagnosis and typing of T cell function a subject or a T cell library.

In some instances, screening noise can be introduced by a soluble TCR secreted polypeptide binding to endogenously express pMHC on the cell. This noise is mitigated using several solutions: 1) using a cell that is engineered to have reduced endogenous display of pMHC using methods known to those skilled in the art (for example, TAP transporter gene knockout, calreticulin knockout, tapasin knockout, HLA gene knockouts); 2) secreting a pMHC as the secreted polypeptide, with a TCR as the membrane-bound target. In some cases, screening noise is reduced by first expressing a soluble TCR library as the secreted polypeptide in the absence of an engineered membrane-bound target, and removing all binding TCRs from the cell library before the membrane-bound target pMHC antigens are expressed. This removes any endogenous cell-binding TCRs from the library before membrane-bound target pMHC are screened.

All references provided in this disclosure, including those listed below, are incorporated herein by reference in their entireties.
1. Mortensen R M, Kingston R E. Selection of transfected mammalian cells. Curr Protoc Mol Biol. 2009 April; Chapter 9:Unit9.5. doi: 10.1002/0471142727.mb0905s86. PMID: 19343709.DeKosky, B. J., Kojima, T., Rodin, A., Charab, W., Ippolito, G. C., Ellington, A. D. & Georgiou, G. In-depth determination and analysis of the human paired heavy- and light-chain antibody repertoire. Nat. Med. 21, 86-91 (2015).
2. Wang, B., DeKosky, B. J., Timm, M. R., Lee, J., Normandin, E., Misasi, J., Kong, R., McDaniel, J. R., Delidakis, G., Leigh, K. E., Niezold, T., Choi, C. W., Viox, E. G., Fahad, A., Cagigi, A., Ploquin, A., Leung, K., Yang, E. S., Kong, W.-P., Voss, W. N., Schmidt, A. G., Moody, M. A., Ambrozak, D. R., Henry, A. R., Laboune, F., Ledgerwood, J. E., Graham, B. S., Connors, M., Douek, D. C., Sullivan, N. J., Ellington, A. D., Mascola, J. R. & Georgiou, G. Functional interrogation and mining of natively paired human VH:VL antibody repertoires. Nature Biotechnology 36, 152-155 (2018).
3. DeKosky, B. J., Ippolito, G. C., Deschner, R. P., Lavinder, J. J., Wine, Y., Rawlings, B. M., Varadarajan, N., Giesecke, C., Dorner, T., Andrews, S. F., Wilson, P. C., Hunicke-Smith, S. P., Willson, C. G., Ellington, A. D. & Georgiou, G. High-throughput sequencing of the paired human immunoglobulin heavy and light chain repertoire. Nat Biotech 31, 166-169 (2013).
4. https://www.berkeleylights.com/systems/beacon/
5. Lagerman, C. E., López Acevedo, S. N., Fahad, A. S., Hailemariam, A. T., Madan, B. & DeKosky, B. J. Ultrasonically-guided flow focusing generates precise emulsion droplets for high-throughput single cell analyses. Journal of Bioscience and Bioengineering 128, 226-233 (2019).
6. Georgina To'a Salazar, Ziyi Huang, Ningyan Zhang, Xue-Guang Zhang, Zhiqiang An, Antibody Therapies Targeting Complex Membrane Proteins, Engineering, Volume 7, Issue 11, 2021, Pages 1541-1551, ISSN 2095-8099.
7. Hoffmann, M., Kleine-Weber, H., Schroeder, S., Kruger, N., Herrler, T., Erichsen, S., Schiergens, T. S., Herrler, G., Wu, N.-H., Nitsche, A., Müller, M. A., Drosten, C. & Pohlmann, S. SARS-CoV-2 Cell Entry Depends on ACE2 and TMPRSS2 and Is Blocked by a Clinically Proven Protease Inhibitor. Cell 181, 271-280.e8 (2020).
8. Nat Protoc. 2016 March; 11(3):429-42. doi: 10.1038/nprot.2016.024. Epub 2016 Feb. 4.
9. Kulmala A, Huovinen T, Lamminmaki U. Improvement of Fab expression by screening combinatorial synonymous signal sequence libraries. Microb Cell Fact. 2019 Sep. 16; 18(1):157. doi: 10.1186/s12934-019-1210-1. PMID: 31526395; PMCID: PMC6745802.
10. Zalucki Y M, Beacham I R, Jennings M P. Biased codon usage in signal peptides: a role in protein export. Trends Microbiol. 2009 April; 17(4):146-50. doi: 10.1016/j.tim.2009.01.005. Epub 2009 Mar. 21. PMID: 19307122.
11. Arnold C E, Parekh R N, Yang W, Wittrup K D. Leader peptide efficiency correlates with signal recognition particle dependence in Saccharomyces cerevisiae. Biotechnol Bioeng. 1998 Aug. 5; 59(3):286-93. PMID: 10099339.
12. Khorshidi S, Zomorodipour A, Behmanesh M, Vatandoost J, Bos M H. Functional expression of the human coagulation factor IX using heterologous signal peptide and propeptide sequences in mammalian cell line. Biotechnol Lett. 2015 September; 37(9):1773-81. doi: 10.1007/s10529-015-1868-3. Epub 2015 Jun. 24. Erratum in: Biotechnol Lett. 2015 October; 37(10):2097. PMID: 26105559.
13. Onu A, Pohl T, Krause H, Bulfone-Paus S. Regulation of IL-15 secretion via the leader peptide of two IL-15 isoforms. J Immunol. 1997 Jan. 1; 158(1):255-62. PMID: 8977197.Mukhamedova, M. et al. Vaccination with prefusion-stabilized respiratory syncytial virus fusion protein induces genetically and antigenically diverse antibody responses. Immunity 0, (2021).
14. Fahad, A. S. et al. Functional Profiling of Antibody Immune Repertoires in Convalescent Zika Virus Disease Patients. Front. Immunol. 12, (2021).
15. Banach, B. B. et al. Paired heavy and light chain signatures contribute to potent SARS-CoV-2 neutralization in public antibody responses. bioRxiv 2020.12.31.424987 (2021) doi:10.1101/2020.12.31.424987.
16. DeKosky, B. J. et al. In-depth determination and analysis of the human paired heavy- and light-chain antibody repertoire. Nat. Med. 21, 86-91 (2015).
17. Wang, B. et al. Functional interrogation and mining of natively paired human VH:VL antibody repertoires. Nature Biotechnology 36, 152-155 (2018).
18. Tian, M. et al. Induction of HIV Neutralizing Antibody Lineages in Mice with Diverse Precursor Repertoires. Cell 166, 1471-1484.e18 (2016).
19. Jin, W., Madan, B., Mussman, B. K., Hailemariam, A. T., Fahad, A. S., Wolfe, J. R., Kwon, Y. D., Zhang, B., Shapiro, L., Kwong, P. D. & DeKosky, B. J. The Covalent SNAP Tag for Protein Display Quantification and Low-pH Protein Engineering. Journal of Biotechnology (2020). doi:10.1016/j.jbiotec.2020.06.011
20. Georgiou, G., Ippolito, G. C., Beausang, J., Busse, C. E., Wardemann, H. & Quake, S. R. The promise and challenge of high-throughput sequencing of the antibody repertoire. Nature Biotechnology 32, 158-168 (2014).
21. Gludish D W, Boliar S, Caldwell S, Tembo D L, Chimbayo E T, Jambo K C, Mwandumba H C, Russell D G. TZM-gfp cells: a tractable fluorescent tool for analysis of rare and early HIV-1 infection. Sci Rep. 2020 Nov. 16; 10(1):19900. doi: 10.1038/s41598-020-76422-6. PMID: 33199722; PMCID: PMC7670428.
22. Abate A R, Hung T, Mary P, Agresti J J, Weitz D A. High-throughput injection with microfluidics using picoinjectors. Proc Natl Acad Sci USA. 2010 Nov. 9; 107(45):19163-6. doi: 10.1073/pnas.1006888107. Epub 2010 Oct. 20. PMID: 20962271; PMCID: PMC2984161.Seah, Y. F. S., Hu, H. & Merten, C. A. Microfluidic single-cell technology in immunology and antibody screening. Molecular Aspects of Medicine 59, 47-61 (2018).
23. Debs, B. E., Utharala, R., Balyasnikova, I. V., Griffiths, A. D. & Merten, C. A. Functional single-cell hybridoma screening using droplet-based microfluidics. Proceedings of the National Academy of Sciences 109, 11570-11575 (2012).
24. Glanville, J., Kuo, T. C., von Budingen, H.-C., Guey, L., Berka, J., Sundar, P. D., Huerta, G., Mehta, G. R., Oksenberg, J. R., Hauser, S. L., Cox, D. R., Rajpal, A. & Pons, J. Naive antibody gene-segment frequencies are heritable and unaltered by chronic lymphocyte ablation. Proc Natl Acad Sci USA 108, 20066-20071 (2011).
25. Dodd, R. B., Wilkinson, T. & Schofield, D. J. Therapeutic Monoclonal Antibodies to Complex Membrane Protein Targets: Antigen Generation and Antibody Discovery Strategies. BioDrugs 32, 339-355 (2018).
26. Hutchings, C. J. Mini-review: antibody therapeutics targeting G protein-coupled receptors and ion channels. Antibody Therapeutics 3, 257-264 (2020).
27. Ren, H., Li, J., Zhang, N., Hu, L. A., Ma, Y., Tagari, P., Xu, J. & Zhang, M.-Y. Function-based high-throughput screening for antibody antagonists and agonists against G protein-coupled receptors. Commun Biol 3, 1-10 (2020).
28. To'a Salazar, G., Huang, Z., Zhang, N., Zhang, X.-G. & An, Z. Antibody Therapies Targeting Complex Membrane Proteins. Engineering 7, 1541-1551 (2021).
29. Dodd, R. B., Wilkinson, T. & Schofield, D. J. Therapeutic Monoclonal Antibodies to Complex Membrane Protein Targets: Antigen Generation and Antibody Discovery Strategies. BioDrugs 32, 339-355 (2018).
30. Hutchings, C. J. Mini-review: antibody therapeutics targeting G protein-coupled receptors and ion channels. Antibody Therapeutics 3, 257-264 (2020).
31. Ren, H., Li, J., Zhang, N., Hu, L. A., Ma, Y., Tagari, P., Xu, J. & Zhang, M.-Y. Function-based high-throughput screening for antibody antagonists and agonists against G protein-coupled receptors. Commun Biol 3, 1-10 (2020).
32. To'a Salazar, G., Huang, Z., Zhang, N., Zhang, X.-G. & An, Z. Antibody Therapies Targeting Complex Membrane Proteins. Engineering 7, 1541-1551 (2021).
33. Bournazos, S., Gupta, A. & Ravetch, J. V. The role of IgG Fc receptors in antibody-dependent enhancement. Nature Reviews Immunology 1-11 (2020). doi:10.1038/s41577-020-00410-0.
34. Merle, N. S., Church, S. E., Fremeaux-Bacchi, V., & Roumenina, L. T. (1AD, January 1). Complement system part I—molecular mechanisms of activation and regulation.
35. Dodd, R. B., Wilkinson, T. & Schofield, D. J. Therapeutic Monoclonal Antibodies to Complex Membrane Protein Targets: Antigen Generation and Antibody Discovery Strategies. BioDrugs 32, 339-355 (2018).
36. Hutchings, C. J. Mini-review: antibody therapeutics targeting G protein-coupled receptors and ion channels. Antibody Therapeutics 3, 257-264 (2020).
37. Ren, H., Li, J., Zhang, N., Hu, L. A., Ma, Y., Tagari, P., Xu, J. & Zhang, M.-Y. Function-based high-throughput screening for antibody antagonists and agonists against G protein-coupled receptors. Commun Biol 3, 1-10 (2020).
38. To'a Salazar, G., Huang, Z., Zhang, N., Zhang, X.-G. & An, Z. Antibody Therapies Targeting Complex Membrane Proteins. Engineering 7, 1541-1551 (2021).
39. Huang, A., Jin, W., Fahad, A. S., Mussman, B. K., Nicchia, G. P., Madan, B., de Souza, M. O., Griffin, J. D., Bennett, J. L., Frigeri, A., Berkland, C. J. & DeKosky, B. J. Strategies to Screen Anti-AQP4 Antibodies from Yeast Surface Display Libraries. Antibodies 11, 39 (2022).
40. Setliff, I., Shiakolas, A. R., Pilewski, K. A., Murji, A. A., Mapengo, R. E., Janowska, K., Richardson, S., Oosthuysen, C., Raju, N., Ronsard, L., Kanekiyo, M., Qin, J. S., Kramer, K. J., Greenplate, A. R., McDonnell, W. J., Graham, B. S., Connors, M., Lingwood, D., Acharya, P., Morris, L. & Georgiev, I. S. High-Throughput Mapping of B Cell Receptor Sequences to Antigen Specificity. Cell 179, 1636-1646.e15 (2019).
41. Shiakolas, A. R., Kramer, K. J., Johnson, N. V., Wall, S. C., Suryadevara, N., Wrapp, D., Periasamy, S., Pilewski, K. A., Raju, N., Nargi, R., Sutton, R. E., Walker, L. M., Setliff, I., Crowe, J. E., Bukreyev, A., Carnahan, R. H., McLellan, J. S. & Georgiev, I. S. Efficient discovery of SARS-CoV-2-neutralizing antibodies via B cell receptor sequencing and ligand blocking. Nat Biotechnol (2022). doi:10.1038/s41587-022-01232-2
42. Huang, A., Jin, W., Fahad, A. S., Mussman, B. K., Nicchia, G. P., Madan, B., de Souza, M. O., Griffin, J. D., Bennett, J. L., Frigeri, A., Berkland, C. J. & DeKosky, B. J. Strategies to Screen Anti-AQP4 Antibodies from Yeast Surface Display Libraries. Antibodies 11, 39 (2022).
43. Cho, Y. K. & Shusta, E. V. Antibody library screens using detergent-solubilized mammalian cell lysates as antigen sources. Protein Eng Des Sel 23, 567-577 (2010).
44. Tillotson, B. J., Cho, Y. K. & Shusta, E. V. Cells and Cell Lysates: A Direct Approach for Engineering Antibodies Against Membrane Proteins Using Yeast Surface Display. Methods 60, 27-37 (2013).
45. Umlauf, B. J., Shusta, E. V., Kuo, J. S., Zoriak, M., Clark, P. A. & Cho, Y. Yeast display biopanning identifies human antibodies targeting glioblastoma stem-like cells. Scientific Reports 7, 15840 (2017).
46. Wang, X. X., Cho, Y. K. & Shusta, E. V. Mining a Yeast Library for Brain Endothelial Cell-Binding Antibodies. Nat Methods 4, 143-145 (2007).
47. Bradbury, A. R. M., Sidhu, S., Dubel, S. & McCafferty, J. Beyond natural antibodies: the power of in vitro display technologies. Nature Biotechnology 29, 245-254 (2011).
48. Georgiou, G., Stathopoulos, C., Daugherty, P. S., Nayak, A. R., Iverson, B. L. & Iii, R. C. Display of heterologous proteins on the surface of microorganisms: From the screening of combinatorial libraries to live recombinant vaccines. Nat Biotechnol 15, 29-34 (1997).
49. Valldorf, B., Hinz, S. C., Russo, G., Pekar, L., Mohr, L., Klemm, J., Doemer, A., Krah, S., Hust, M. & Zielonka, S. Antibody display technologies: selecting the cream of the crop. Biological Chemistry 403, 455-477 (2022).
50. Sakurai, T. & Iizuka, Ryo & Sekine, R. & Nakamura, Yasuyuki & Yoon, Dong Hyun & Sekiguchi, Tetsushi & Ishii, Jun & Kondo, A. & Shoji, S. & Funatsu, Takashi. (2014). Peptide-based ligand screening system for g protein-coupled receptors using water-in-oil microdroplets. 18th International Conference on Miniaturized Systems for Chemistry and Life Sciences, MicroTAS 2014. 965-967.
51. Mazutis L, Gilbert J, Ung W L, Weitz D A, Griffiths A D, Heyman J A. Single-cell analysis and sorting using droplet-based microfluidics. Nat Protoc. 2013 May; 8(5):870-91. doi: 10.1038/nprot.2013.046. Epub 2013 Apr. 4. PMID: 23558786; PMCID: PMC4128248.
52. Eyer, K., Doineau, R. C. L., Castrillon, C. E., Briseño-Roa, L., Menrath, V., Mottet, G., England, P., Godina, A., Brient-Litzler, E., Nizak, C., Jensen, A., Griffiths, A. D., Bibette, J., Bruhns, P. & Baudry, J. Single-cell deep phenotyping of IgG-secreting cells for high-resolution immune monitoring. Nature Biotechnology 35, nbt.3964 (2017).
53. Gérard, A., Woolfe, A., Mottet, G., Reichen, M., Castrillon, C., Menrath, V., Ellouze, S., Poitou, A., Doineau, R., Briseno-Roa, L., Canales-Herrerias, P., Mary, P., Rose, G., Ortega, C., Delincé, M., Essono, S., Jia, B., Iannascoli, B., Goff, O. R.-L., Kumar, R., Stewart, S. N., Pousse, Y., Shen, B., Grosselin, K., Saudemont, B., Sautel-Caille, A., Godina, A., McNamara, S., Eyer, K., Millot, G. A., Baudry, J., England, P., Nizak, C., Jensen, A., Griffiths, A. D., Bruhns, P. & Brenan, C. High-throughput single-cell activity-based screening and sequencing of antibodies using droplet microfluidics. Nature Biotechnology 1-7 (2020). doi:10.1038/s41587-020-0466-7
54. Lin W N, Tay M Z, Wong J X E, Lee C Y, Fong S W, Wang C I, Ng L F P, Renia L, Chen C H, Cheow L F. Rapid microfluidic platform for screening and enrichment of cells secreting virus neutralizing antibodies. Lab Chip. 2022 Jun. 28; 22(13):2578-2589. doi: 10.1039/d21c00018k. PMID: 35694804.
55. Yaginuma, K., Aoki, W., Miura, N. et al. High-throughput identification of peptide agonists against GPCRs by co-culture of mammalian reporter cells and peptide-secreting yeast cells using droplet microfluidics. Sci Rep 9, 10920 (2019).
56. Ye Jin et al. Accelerated Discovery of Unique Anti-PD-L1 Antibodies from Spleen Versus Bone Marrow of Immunized Mice by Single Plasma B cell Cloning on the Beacon® Platform.
57. Le, K., Tan, C., Gupta, S., Guhan, T., Barkhordarian, H., Lull, J., Stevens, J. and Munro, T. (2018), A novel mammalian cell line development platform utilizing nanofluidics and optoelectro positioning technology. Biotechnol. Prog., 34: 1438-1446.
58. Van Lent J, Breukers J, Ven K, Ampofo L, Horta S, Pollet F, Imbrechts M, Geukens N, Vanhoorelbeke K, Declerck P, Lammertyn J. Miniaturized single-cell technologies for monoclonal antibody discovery. Lab Chip. 2021 Sep. 28; 21(19):3627-3654. doi: 10.1039/d11c00243k. PMID: 34505611.
59. Younger, D., Berger, S., Baker, D. & Klavins, E. High-throughput characterization of protein-protein interactions by reprogramming yeast mating. PNAS 114, 12166-12171 (2017).
60. Ren, H., Li, J., Zhang, N., Hu, L. A., Ma, Y., Tagari, P., Xu, J. & Zhang, M.-Y. Function-based high-throughput screening for antibody antagonists and agonists against G protein-coupled receptors. Commun Biol 3, 1-10 (2020).
61. Gludish, D. W., Boliar, S., Caldwell, S., Tembo, D. L., Chimbayo, E. T., Jambo, K. C., Mwandumba, H. C. & Russell, D. G. TZM-gfp cells: a tractable fluorescent tool for analysis of rare and early HIV-1 infection. Scientific Reports 10, 19900 (2020).
62. Mkhize, N. N., Yssel, A. E. J., Kaldine, H., Dorsten, R. T. van, Davis, A. S. W., Beaume, N., Matten, D., Lambson, B., Modise, T., Kgagudi, P., York, T., Westfall, D. H., Giorgi, E. E., Korber, B., Anthony, C., Mapengo, R. E., Bekker, V., Domin, E., Eaton, A., Deng, W., DeCamp, A., Huang, Y., Gilbert, P. B., Gwashu-Nyangiwe, A., Thebus, R., Ndabambi, N., Mielke, D., Mgodi, N., Karuna, S., Edupuganti, S., Seaman, M. S., Corey, L., Cohen, M. S., Hural, J., McElrath, M. J., Mullins, J. I., Montefiori, D., Moore, P. L., Williamson, C. & Morris, L. Neutralization profiles of HIV-1 viruses from the VRCO1 Antibody Mediated Prevention (AMP) trials. PLOS Pathogens 19, e1011469 (2023).
63. Zhu, J., Wu, X., Zhang, B., McKee, K., O'Dell, S., Soto, C., Zhou, T., Casazza, J. P., Program, N. C. S., Mullikin, J. C., Kwong, P. D., Mascola, J. R. & Shapiro, L. De novo identification of VRCO1 class HIV-1-neutralizing antibodies by next-generation sequencing of B-cell transcripts. Proceedings of the National Academy of Sciences 110, E4088-97 (2013).
64. Shen, C.-H., DeKosky, B. J., Guo, Y., Xu, K., Gu, Y., Kilam, D., Ko, S. H., Kong, R., Liu, K., Louder, M. K., Ou, L., Zhang, B., Chao, C. W., Corcoran, M. M., Feng, E., Huang, J., Normandin, E., O'Dell, S., Ransier, A., Rawi, R., Sastry, M., Schmidt, S. D., Wang, S., Wang, Y., Chuang, G.-Y., Doria-Rose, N. A., Lin, B., Zhou, T., Boritz, E. A., Connors, M., Douek, D. C., Karlsson Hedestam, G. B., Sheng, Z., Shapiro, L., Mascola, J. R. & Kwong, P. D. VRC34-Antibody Lineage Development Reveals How a Required Rare Mutation Shapes the Maturation of a Broad HIV-Neutralizing Lineage. Cell Host & Microbe 27, 531-543.e6 (2020).
65. Kong, R., Xu, K., Zhou, T., Acharya, P., Lemmin, T., Liu, K., Ozorowski, G., Soto, C., Taft, J. D., Bailer, R. T., Cale, E. M., Chen, L., Choi, C. W., Chuang, G.-Y., Doria-Rose, N. A., Druz, A., Georgiev, I. S., Gorman, J., Huang, J., Joyce, M. G., Louder, M. K., Ma, X., McKee, K., O'Dell, S., Pancera, M., Yang, Y., Blanchard, S. C., Mothes, W., Burton, D. R., Koff, W. C., Connors, M., Ward, A. B., Kwong, P. D. & Mascola, J. R. Fusion peptide of HIV-1 as a site of vulnerability to neutralizing antibody. Science 352, 828-833 (2016).
66. Tanner, J. E., Coingon, M., Leblond, V., Hu, J., Fang, J. M., Sygusch, J. & Alfieri, C. Peptides Designed To Spatially Depict the Epstein-Barr Virus Major Virion Glycoprotein gp350 Neutralization Epitope Elicit Antibodies That Block Virus-Neutralizing Antibody 72A1 Interaction with the Native gp350 Molecule. J. Virol. 89, 4932-4941 (2015).
67. Sairenji, T., Bertoni, G., Medveczky, M. M., Medveczky, P. G., Nguyen, Q. V. & Humphreys, R. E. Inhibition of Epstein-Barr virus (EBV) release from P3HR-1 and B95-8 cell lines by monoclonal antibodies to EBV membrane antigen gp350/220. J. Virol. 62, 2614-2621 (1988).
68. Mkhize, N. N., Yssel, A. E. J., Kaldine, H., Dorsten, R. T. van, Davis, A. S. W., Beaume, N., Matten, D., Lambson, B., Modise, T., Kgagudi, P., York, T., Westfall, D. H., Giorgi, E. E., Korber, B., Anthony, C., Mapengo, R. E., Bekker, V., Domin, E., Eaton, A., Deng, W., DeCamp, A., Huang, Y., Gilbert, P. B., Gwashu-Nyangiwe, A., Thebus, R., Ndabambi, N., Mielke, D., Mgodi, N., Karuna, S., Edupuganti, S., Seaman, M. S., Corey, L., Cohen, M. S., Hural, J., McElrath, M. J., Mullins, J. I., Montefiori, D., Moore, P. L., Williamson, C. & Morris, L. Neutralization profiles of HIV-1 viruses from the VRC01 Antibody Mediated Prevention (AMP) trials. PLOS Pathogens 19, e1011469 (2023).
69. Parsons, M. S., Chung, A. W. & Kent, S. J. Importance of Fc-mediated functions of anti-HIV-1 broadly neutralizing antibodies. Retrovirology 15, 58 (2018).
70. Gunn B M, Lu R, Slein M D, Ilinykh P A, Huang K, Atyeo C, Schendel S L, Kim J, Cain C, Roy V, Suscovich T J, Takada A, Halfmann P J, Kawaoka Y, Pauthner M G, Momoh M, Goba A, Kanneh L, Andersen K G, Schieffelin J S, Grant D, Garry R F, Saphire E O, Bukreyev A, Alter G. A Fc engineering approach to define functional humoral correlates of immunity against Ebola virus. Immunity. 2021 Apr. 13; 54(4):815-828.e5. doi: 10.1016/j.immuni.2021.03.009. PMID: 33852832; PMCID: PMC8111768.
71. Gironda-Martinez A, Donckele E J, Samain F, Neri D. DNA-Encoded Chemical Libraries: A Comprehensive Review with Succesful Stories and Future Challenges. ACS Pharmacol Transl Sci. 2021 Jun. 14; 4(4):1265-1279. doi: 10.1021/acsptsci.1c00118. PMID: 34423264; PMCID: PMC8369695.
72. Stoeckius M, Hafemeister C, Stephenson W, Houck-Loomis B, Chattopadhyay P K, Swerdlow H, Satija R, Smibert P. Simultaneous epitope and transcriptome measurement in single cells. Nat Methods. 2017 September; 14(9):865-868. doi: 10.1038/nmeth.4380. Epub 2017 Jul. 31. PMID: 28759029; PMCID: PMC5669064

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. Any cited references are incorporated by reference herein in their entireties. In the event that there is an inconsistency between a definition of a term in the specification as compared to a definition of the term in a cited reference, the term should be interpreted based on the definition in the specification.

We claim:

1. A screening method comprising:
detecting the presence of binding between a secreted polypeptide and a membrane-bound target,
wherein the secreted polypeptide or the membrane-bound target comprises a polypeptide sequence derived from an antibody or fragment thereof,
wherein the secreted polypeptide and the membrane-bound target are included within a compartment,
wherein the compartment includes a single, isolated genetically engineered cell expressing the secreted polypeptide and presenting the membrane bound target,
wherein the cell is engineered to secrete the secreted polypeptide, present the membrane bound target, or both, and
wherein detecting binding between a secreted polypeptide and the membrane-bound target comprises: providing a detection agent, wherein the detection agent binds to the secreted polypeptide, the membrane-bound target, or both, and wherein the detection agent does not enter the cell.

2. (canceled)

3. The method of claim 1, wherein the detection agent comprises one or more of an antibody Fc effector protein, a complement protein, a ligand, a polypeptide, a chemical, a nucleic acid sequence, an antibody or fragment thereof, and a reporter molecule.

4. (canceled)

5. The method of claim 5, wherein the reporter molecule comprises one or more of a nucleic acid barcode, a dye, a fluorescent molecule, an enzyme, a chemical, a protein, a polypeptide tag.

6. (canceled)

7. The method of claim 1, wherein the secreted polypeptide aid/or the membrane-bound target comprises at least one of a chemical moiety, a polymer, an oligomer, a nucleic acid, and a peptide sequence, optionally a fusion protein.

8. The method of claim 1, wherein the compartment is a well, a droplet, spatially separated cell culture condition, or an encapsulation.

9. The method of claim 1, wherein the secreted polypeptide comprises a reporter molecule.

10. (canceled)

11. The method of claim 1, wherein the method comprises, before or contemporaneous with the detection step, generating a collection of genetically engineered cells, wherein each of the genetically engineered cells comprises a gene encoding a secreted polypeptide from a library of secreted polypeptides.

12. The method of claim 1, wherein the method comprises, before or contemporaneous with the detection step, generating a collection of genetically engineered cells, wherein each of the genetically engineered cells comprises a gene encoding a membrane-bound target from a library of membrane-bound targets.

13. The method of claim 1, wherein the method comprises, before or contemporaneous with the detection step, generating a collection of genetically engineered cells, wherein each of the genetically engineered cells comprises a gene encoding a membrane-bound polypeptide of a library of membrane-bound polypeptides and a gene encoding a secreted polypeptide from a library of secreted polypeptides.

14.-15. (canceled)

16. The method of claim 10, wherein the detection agent comprises another cell or a virus.

17. The method of claim 1, wherein the secreted polypeptide comprises one of a T cell receptor and a peptide:MHC complex.

18. The method claim 1, wherein the membrane-bound target comprises one of a T cell receptor, and a peptide:MHC complex.

19.-30. (canceled)

31. The method of claim 1, wherein the secreted polypeptide comprises a proteolysis targeting chimera (PROTAC).

32. The method of claim 31, wherein the secreted polypeptide comprising the PROTAC comprises an antibody, scFv, VHH, nanobody, TCR, pMHC, Fab, IgG, ligand, or other binding polypeptide.

33. The method of claim 1, wherein one or more of the secreted polypeptide, the membrane-bound target, and the detection agent comprise a reporter molecule.

34. The method of claim 1, wherein the membrane-bound target is bound to or expressed on the cell surface prior to compartmentation.

35. The method of claim 1, wherein the membrane-bound target is bound to or expressed on the cell surface during or after compartmentation.

36. A kit comprising:
(a) a nucleic acid sequence encoding a secreted polypeptide or a cell containing the nucleic acid sequence encoding the secreted peptide; and
(b) a nucleic acid sequence encoding a membrane-bound target, or a cell containing the nucleic acid sequence encoding the membrane-bound target, wherein the secreted polypeptide may be screened for binding to the membrane-bound target;
optionally, wherein the nucleic acid encoding the secreted polypeptide and the nucleic acid encoding the membrane-bound target are present on a same construct.

37. The kit of claim 36, further comprising a detection agent comprising a reporter molecule, wherein the detection agent comprises an antibody Fc effector protein, a complement protein a ligand, a polypeptide, a chemical, a nucleic acid sequence, an antibody or fragment thereof.


SEQUENCE LISTING (34 sequences)

SEQ ID NO: 1 - protein, 238 aa, synthetic construct
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 2 - protein, 237 aa, synthetic construct
MRGSHHHHHHGSAHGLTDDMTMHFRMEGCVDGHKFVIEGNGNGNPFKGKQFINLCVIEGGPLPFSEDILSAAFXNRLFTEYPEGIVDYFKNSCPAGYTWHRSFRFEDGAVCICSADITVNVRENCIYHESTFYGVNFPADGPVMKKMTTNWEPSCEKIIPINSQKILKGDVSMYLLLKDGGRYRCQFDTIYKAKTEPKEMPDWHFIQHKLNREDRSDAKNQKWQLIEHAIASRSALP

SEQ ID NO: 3 - protein, 225 aa, synthetic construct
KGEELFTGVVPILVELDGDVNGHKFSVSGEGEGDATYGKLTLKFICTTGKLPVPWPTLVTTFXLQCFARYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGI

SEQ ID NO: 4 - protein, 236 aa, synthetic construct
MSKGEELFTGVVPILVELDGDVNGHKFSVSGEGEGDATYGKLTLKFICTTGKLPVPWPTLVTTFXVQCFSRYPDHMKRHDFFKSAMPEGYVQERTIFFKDDGNYKTRAEVKFEGDTLVNRIELKGIDFKEDGNILGHKLEYNFNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 5 - protein, 237 aa, synthetic construct
MVSKGEELFTGVVPILVELDGDVNGHKFSVSGEGEGDATYGKLTLKFICTTGKLPVPWPTLVTTLXVQCFARYPDHMKQHDFFKSAMPEGYVQERTIFFKDDGNYKTRAEVKFEGDTLVNRIELKGIDFKEDGNILGHKLEYNAISDNVYITADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 6 - protein, 237 aa, synthetic construct
MVSKGEELFTGVVPILVELDGDVNGHKFSVSGEGEGDATYGKLTLKFICTTGKLPVPWPTLVTTLXVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 7 - protein, 236 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 8 - protein, 311 aa, synthetic construct
MTSKVYDPELRKRMITGPQWWARCKQMNVLDSFINYYDSEKHAENAVIFLHGNAASSYLWRHVVPHVEPVARCIIPDLIGMGKSGKSGNGSYRLLDHYKYLTEWFKHLNLPKKIIFVGHDWGACLAFHYCYEHQDRIKAVVHAESVVDVIESWDEWPDIEEDIALIKSEEGEKMVLENNFFVETMLPSKIMRKLEPEEFAAYLEPFKEKGEVRRPTLSWPREIPLVKGGKPDVVEIVRNYNAYLRASHDLPKMFIESDPGFFSNAIVEGAKKFPNTEFVKVKGLHFSQEDAPDEMGNYIKSFVERVLKNEQ

SEQ ID NO: 9 - protein, 550 aa, Photinus pyralis
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGSPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNDPEATNALIDKDGWLHSGDIAYWDEDEHFFIVDRLKSLIKYKGCQVAPAELESILLQHPNIFDAGVAGLPGDDAGELPAAVVVLEHGKTMTEKEIVDYVASQVTTAKKLRGGVVFVDEVPKGLTGKLDARKIREILIKAKKGGKSKL

SEQ ID NO: 10 - protein, 805 aa, Homo sapiens [sequence omitted]

SEQ ID NO: 11 - protein, 550 aa, Homo sapiens; identical to SEQ ID NO: 9

SEQ ID NO: 12 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 13 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 14 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 15 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 16 - protein, 492 aa, Homo sapiens
MALNSGSPPAIGPYYENHGYQPENPYPAQPTVVPTVYEVHPAQYYPSPVPQYAPRVLTQASNPVVCTQPKSPSGTVCTSKTKKALCITLTLGTFLVGAALAAGLLWKFMGSKCSNSGIECDSSGTCINPSNWCDGVSHCPGGEDENRCVRLYGPNFILQVYSSQRKSWHPVCQDDWNENYGRAACRDMGYKNNFYSSQGIVDDSGSTSFMKLNTSAGNVDIYKKLYHSDACSSKAVVSLRCIACGVNLNSSRQSRIVGGESALPGAWPWQVSLHVQNVHVCGGSIITPEWIVTAAHCVEKPLNNPWHWTAFAGILRQSFMFYGAGYQVEKVISHPNYDSKTKNNDIALMKLQKPLTFNDLVKPVCLPNPGMMLQPEQLCWISGWGATEEKGKTSEVLNAAKVLLIETQRCNSRYVYDNLITPAMICAGFLQGNVDSCQGDSGGPLVTSKNNIWWLIGDTSWGSGCAKAYRPGVYGNVMVFTDWIYRQMRADG

SEQ ID NO: 17 - protein, 25 aa, synthetic construct
MSKRGASNEMALLLLLLGLLQQAMA

SEQ ID NO: 18 - DNA, 75 bp, synthetic construct
  source 1..75: mol_type = other DNA
atgagcaagaggggcgccagcaacgagatggccctgctgctgctgctgctgggcctgctgcagcaggccatggcg

SEQ ID NO: 19 - protein, 18 aa, synthetic construct
MKWVTFISLLFLFSSAMA

SEQ ID NO: 20 - DNA, 54 bp, synthetic construct
  source 1..54: mol_type = other DNA
atgaagtgggtgaccttcatcagcctgctgttcctgttcagcagcgccatggcg

SEQ ID NO: 21 - protein, 25 aa, synthetic construct
MSKRGASNEMALLLLLLGLLASVLA

SEQ ID NO: 22 - DNA, 74 bp, synthetic construct
  source 1..74: mol_type = other DNA
atgagcaagaggggcgccagcaacgagatggccctgctgctgctgctgctgggcctgctggdagcgttttagca

SEQ ID NO: 23 - protein, 18 aa, synthetic construct
MKWVTFISLLFLFASVLA

SEQ ID NO: 24 - DNA, 53 bp, synthetic construct
  source 1..53: mol_type = other DNA
atgaagtgggtgaccttcatctccctgcttgcctgttcgctagcgttttagca

SEQ ID NO: 25 - DNA, 20 bp, synthetic construct
  source 1..20: mol_type = other DNA
tcctgagtccggaccacttt

SEQ ID NO: 26 - DNA, 22 bp, synthetic construct
  source 1..22: mol_type = other DNA
caccgcatgttagaagacttcc

SEQ ID NO: 27 - DNA, 23 bp, synthetic construct
  source 1..23: mol_type = other DNA
acctccagttaggaaaggggact

SEQ ID NO: 28 - DNA, 25 bp, synthetic construct
  source 1..25: mol_type = other DNA
aagtttttcttgaaaacccatggaa

SEQ ID NO: 29 - DNA, 21 bp, synthetic construct
  source 1..21: mol_type = other DNA
ccttgtagatgaactcgccgt

SEQ ID NO: 30 - DNA, 22 bp, synthetic construct
  source 1..22: mol_type = other DNA
ggtgtcgtgattattcgaaggg

SEQ ID NO: 31 - DNA, 21 bp, synthetic construct
  source 1..21: mol_type = other DNA
ggtgggaggtctatataagca

SEQ ID NO: 32 - DNA, 18 bp, synthetic construct
  source 1..18: mol_type = other DNA
ccagaggtgctcttggag

SEQ ID NO: 33 - protein, 22 aa, synthetic construct
MKYLLPTAAAGLLLLAAQPAMA

SEQ ID NO: 34 - DNA, 66 bp, synthetic construct
  source 1..66: mol_type = other DNA
atgaagtatttgttgccgacggcggcggcggggttgttgttgttggcggcgcagccggccatggcg